(19) United States
(12) Patent Application Publication
(10) Pub. No.: US 2025/0353887 A1
(43) Pub. Date: Nov. 20, 2025

(54) Keratin Protein Extracts, Compositions, Methods and Uses Thereof

(71) Applicant: StimLabs LLC, Roswell, GA (US)

(72) Inventors: Rachael Parker, Alpharetta, GA (US); Sarah Griffiths, Atlanta, GA (US); Annelise Mowry Roy, Cumming, GA (US); Maryam Nuru, Sandy Spring, GA (US); Brian Wicker, Atlanta, GA (US)

(73) Assignee: StimLabs LLC, Roswell, GA (US)

(21) Appl. No.: 18/868,408

(22) PCT Filed: May 25, 2023
(86) PCT No.: PCT/US2023/023496

§ 371 Date: Nov. 22, 2024

Related U.S. Application Data
(60) Provisional application No. 63/346,725, filed on May 27, 2022.

(51) Int. Cl. C07K 14/47; A61K 38/00; A61K 45/06; C07K 1/36

(52) U.S. Cl. CPC C07K 14/4741; A61K 45/06; C07K 1/36; A61K 38/00

(57) ABSTRACT

The present application relates to keratin protein extracts, compositions, methods and uses thereof. In some embodiments, the keratin protein extract is a beta-keratin extract. In some embodiments, the compositions are beta-keratin compositions. In some embodiments, the methods are methods of cleaning a keratin source material, methods of extracting beta-keratin, methods of preparing keratin compositions, and methods of using keratin protein extracts and compositions.

### CROSS-REFERENCE TO RELATED APPLICATIONS

The application claims priority to U.S. Provisional Application No. 63/346,725 filed on May 27, 2022, the disclosure of which is incorporated herein by reference in its entirety.

### FIELD

The present application relates to keratin protein extracts, compositions, methods and uses thereof.

### BACKGROUND

Keratin filaments are found in the epidermis and epidermal appendages of certain species. Keratin monomers form alpha-helices or beta-sheets and assemble to form higher level keratin structures such as oligomers, including dimers and tetramers, which then further assemble to form elongated filaments.

Keratin filaments are broken into two categories: alpha-keratin and beta-keratin. Alpha-keratin filaments are found in wool, hair and skin of vertebrates. Alpha-keratin monomers have a molecular weight of about 40-70 kDa, depending on the source material. Alpha-keratin filaments are characterized by an alpha-helical structure. Beta-keratin filaments are found in cornified epidermal appendages of reptiles and birds (e.g., claws, scales, beaks and feathers). Beta-keratin monomers generally have a molecular weight of less than about 40 kDa depending on the source material. For example, beta-keratin monomers extracted from feathers are approximately 10 kDa; claws, beaks and scale beta-keratin monomers are about 32 kDa. Beta-keratin monomers have similar sequence structure and homology across species. See, e.g., Fraser et al. Journal of Structural Biology 209 (2020) 107413. Beta-keratin filaments are characterized by a beta-sheet structure.

Alpha-keratin sources have keratin associated proteins (“KAPs”) which interact with the alpha-keratin monomers and aid in network formation by crosslinking with the oligomers. Beta-keratin sources, by contrast, lack KAPs and instead the beta-keratin monomers, which have a high amount of cysteine, form intermolecular crosslinks through thiol crosslinking to form disulfide bonds creating a denser network and more rigid structures than alpha-keratin networks and structures.

While alpha-keratin containing products dominate the field of biomaterials, beta-keratin extracts and products are also of interest. However, development of beta-keratin containing products presents certain problems compared to alpha-keratin containing products. For example, as discussed above, beta-keratin monomers form a denser network and more rigid structures than alpha-keratin monomers making extraction of beta-keratin monomers different and potentially more difficult than extraction of alpha-keratin monomers. Further, while different beta-keratin extraction methods are known in the art, the resultant beta-keratin extracts are not soluble in neutral and near neutral pH solvents. Instead, beta-keratin extracts often require the use of strong acids or bases, which may cause skin irritation or a decrease in overall biocompatibility of a beta-keratin containing biomaterial. Additionally, the use of strong acids or bases may negatively alter the protein monomers and oligomers (and consequently the protein network) and subsequently inhibit the performance of the biomaterial. Accordingly, there exists a need for efficient extraction methods of beta-keratin that may be solubilized in neutral or near neutral pH solvents.

In addition, because beta-keratin must be extracted from reptiles and birds, the products must be carefully cleaned to eliminate contaminates. Contaminates include animal DNA, tissue and blood as well as bioburden, endotoxins, debris, dirt, non-keratin proteins, other extracellular matrix components, microorganisms, lipids, inorganic matter, other organic matter, pigments, and/or cells. During their studies, the inventors surprisingly discovered that at least some beta-keratin sources, such as feathers, retain a high level of endotoxins, even after washing the source material. High concentrations of endotoxins can lead to serious complications such as disseminated intravascular coagulation, endotoxin shock and adult respiratory distress syndrome. In fact, to meet current FDA requirements, medical device products must contain no more than 20 endotoxin units (EU) per device. Similarly, for biological products, the FDA limits the endotoxin levels to 5 EU per kg body weight. Thus, for the average patient (80 kg), the EU must be less than about 400 EU. To date there are no beta-keratin containing products that are approved or cleared for use by the FDA. Thus, there exists a need for methods of cleaning and extracting beta-keratin source materials to provide beta-keratin that can be safely used as biomaterials.

Finally, keratin monomers, oligomers, and filaments can be used to form biomaterials, such as scaffolds, sheets, nanoparticles, and hydrogels. Keratin containing biomaterials have been shown to be beneficial to promote ideal cellular behavior (Esparza et al., Materials Science and Engineering C 90 (2018) 446-453). As biomaterials, keratin containing products can deliver one or more agents (e.g., nutrients, drugs, biomolecules, etc.) to cells. The cellular environment, which generally includes fluids, proteases and reactive oxygen species, typically breaks down a keratin containing biomaterial to release keratin and other associated agents. However, there should be a balance between the rate of degradation of the keratin containing biomaterial and/or the release of keratin and other agents from the biomaterial and the ability of the biomaterial to remain intact long enough to gradually release all the agents over an extended period of time. As such, there exists a need for beta-keratin containing products that provide appropriate degradation rates for extended release of keratin and other associated agents.

### SUMMARY

Embodiment 1. A protein extract comprising beta-keratin monomers, wherein the extract has less than about 10.0 endotoxin units per mg of extract, such as less than about 0.80 endotoxin units per mg of extract.

Embodiment 2. A protein extract comprising beta-keratin monomers, wherein the extract has about 0.50 mM thiol groups when measured at about 0.1% weight extract per volume using Ellman's assay or above 0.20 mM thiol groups when measured at about 1% weight extract per volume using Ellman's assay.

Embodiment 3. A protein extract comprising beta-keratin monomers, wherein the extract when dehydrated is soluble or slightly soluble in a solvent having a pH from about 7 to about 8 at about 2.5% weight per volume.

Embodiment 4. The protein extract according to any one of embodiments 1-2, wherein the extract when dehydrated is soluble or slightly soluble in a solvent having a pH from about 7 to about 8 at about 2.5% weight per volume.

Embodiment 5. The protein extract according to embodiment 1 or 4, wherein the extract has about 0.50 mM thiol groups when measured at about 0.1% weight per volume using Ellman's assay or above 0.20 mM thiol groups when measured at about 1% weight extract per volume using Ellman's assay.

Embodiment 6. The protein extract according to any one of embodiments 1-5, wherein the extract has less than about 8.0 endotoxin units per mg of extract, such as less than about 0.10 endotoxin units per mg of extract.

Embodiment 7. The protein extract according to any one of embodiments 1-6, wherein the extract is lyophilized.

Embodiment 8. The protein extract according to any one of embodiments 3-7, wherein the extract is soluble and the solvent is water.

Embodiment 9. The protein extract according to any one of embodiments 1-8, wherein the extract when dehydrated is soluble in water at about 5% weight per volume.

Embodiment 10. The protein extract according to any one of embodiments 3-7 and 9, wherein the solvent having a pH from about 7 to about 8 is chosen from water and a PBS buffer having a pH from about 7 to about 8.

Embodiment 11. The protein extract according to any one of embodiments 1-10, wherein the extract comprises beta-keratin oligomers.

Embodiment 12. The protein extract according to any one of embodiments 1-11, wherein the extract does not comprise cysteine modified keratin monomers, oligomers, and/or filaments.

Embodiment 13. A method of extracting beta-keratin monomers comprising mixing a beta-keratin source material with an extraction solution comprising urea at a concentration of at least about 1 M and thioglycolic acid.

Embodiment 14. The method according to embodiment 13, wherein the urea concentration is at least about 5 M.

Embodiment 15. The method according to embodiment 13 or 14, wherein the urea concentration is from about 5 M to about 9 M.

Embodiment 16. The method according to any one of embodiments 13-15, wherein the urea concentration is from about 7 M to about 8 M.

Embodiment 17. The method according to any one of embodiments 13-16, wherein the urea concentration is about 8 M.

Embodiment 18. The method according to any one of embodiments 13-17, wherein the thioglycolic acid is at a concentration of about 0.5 M to about 1.5 M.

Embodiment 19. The method according to any one of embodiments 13-18, wherein the thioglycolic acid is at a concentration of about 1.3 M.

Embodiment 20. The method according to any one of embodiments 13-19, wherein the pH of the extraction solution is from about 7 to about 10.

Embodiment 21. The method according to any one of embodiments 13-20, wherein the pH of the extraction solution is about 10.

Embodiment 22. The method according to any one of embodiments 13-21, wherein the ratio of beta-keratin source material to extraction solution is, or is equivalent to, from about 1 g to about 100 g dry weight beta-keratin source material per about 1000 mL extraction solution.

Embodiment 23. The method according to any one of embodiments 13-22, wherein the ratio of beta-keratin source material to extraction solution is, or is equivalent to, about 15 g dry weight beta-keratin source material per about 1000 mL extraction solution.

Embodiment 24. The method according to any one of embodiments 13-23, wherein the extraction solution further comprises a chelating agent.

Embodiment 25. The method according to any one of embodiments 13-24, wherein the beta-keratin source material is mixed with the extraction solution for at least about 30 minutes.

Embodiment 26. The method according to any one of embodiments 13-25, wherein the temperature of the mixture of the beta-keratin source material and the extraction solution is from about room temperature to about 80° C.

Embodiment 27. The method according to any one of embodiments 13-26, wherein the method further comprises washing the beta-keratin source material with a washing solution and/or sterilizing the beta-keratin source material prior to mixing the beta-keratin source material with the extraction solution.

Embodiment 28. The method according to any one of embodiments 13-27, wherein the method further comprises defatting the beta-keratin source material prior to mixing the beta-keratin source material with the extraction solution.

Embodiment 29. The method according to embodiment 27 or 28, wherein the washing solution comprises at least one detergent.

Embodiment 30. The method according to embodiment 29, wherein the at least one detergent comprises SDS and/or a nonionic detergent.

Embodiment 31. The method according to embodiment 30, wherein the nonionic detergent comprises a polyethylene glycol-based detergent.

Embodiment 32. The method according to embodiment 29 or 30, wherein the nonionic detergent comprises a polyethylene glycol tert-octylphenyl ether.

Embodiment 33. The method according to any one of embodiments 30-32, wherein the nonionic detergent comprises a compound of formula I:

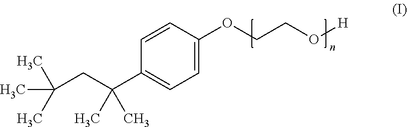

wherein n is chosen from 7-8, 9-10 or 40.

Embodiment 34. The method according to embodiment 33, wherein n is 7-8.

Embodiment 35. The method according to embodiment 33, wherein n is 9-10.

Embodiment 36. The method according to any one of embodiments 13-35, wherein the beta-keratin source material comprises an avian source material, for example goose and/or chicken feathers, and/or a reptilian source material, for example alligator scales.

Embodiment 37. The method according to any one of embodiments 13-36, wherein the extracted beta-keratin protein has less than about 10.0 endotoxin units per mg of extracted beta-keratin protein, such as less than about 0.80 endotoxin units per mg of extracted beta-keratin protein.

Embodiment 38. The method according to any one of embodiments 13-37, wherein the extracted beta-keratin protein has about 0.50 mM free thiol groups when measured at about 0.1% weight per volume using Ellman's assay or above 0.20 mM thiol groups when measured at about 1% weight extract per volume using Ellman's assay.

Embodiment 39. The method according to any one of embodiments 13-38, wherein the extracted beta-keratin protein when dehydrated is soluble or slightly soluble in a solvent having a pH from about 7 to about 8 at about 2.5% weight per volume.

Embodiment 40. The method according to embodiment 39, wherein the extract is soluble and the solvent is water.

Embodiment 41. The method according to any one of embodiments 13-40, wherein the extracted beta-keratin protein is produced at a yield from about 10% to about 70%, for example from about 30% to about 40% yield.

Embodiment 42. A method of preparing a beta-keratin protein extract comprising filtering the product of any one of embodiments 13-41 to produce a filtrate.

Embodiment 43. The method according to embodiment 42, wherein the method further comprises dialyzing the filtrate to produce a dialyzed material.

Embodiment 44. The method according to embodiment 42 or 43, wherein the method further comprises lyophilizing the filtrate or the dialyzed material to produce a lyophilized material.

Embodiment 45. The method according to embodiment 44, wherein the method further comprises homogenizing the lyophilized material.

Embodiment 46. The method according to any one of embodiments 13-45, wherein the method does not include modifying the cysteine residues of the beta-keratin source material and/or the extracted beta-keratin material.

Embodiment 47. A beta-keratin protein extract prepared according to any one of embodiments 13-46, wherein the beta-keratin protein extract has less than about 10.0 endotoxin units per mg of extract, such as less than about 0.80 endotoxin units per mg of extracted beta-keratin protein.

Embodiment 48. A beta-keratin protein extract prepared according to the method of any one of embodiments 13-47, wherein the beta-keratin protein extract has about 0.50 mM free thiol groups when measured at about 0.1% weight per volume using Ellman's assay or above 0.20 mM thiol groups when measured at about 1% weight extract per volume using Ellman's assay.

Embodiment 49. A beta-keratin protein extract prepared according to any one of embodiments 13-48, wherein the beta-keratin protein extract when dehydrated is soluble or slightly soluble in a solvent having a pH from about 7 to about 8 at about 2.5% weight per volume.

Embodiment 50. The beta-keratin protein extract according to embodiment 49, wherein the extract is soluble and the solvent is water.

Embodiment 51. A keratin composition comprising a beta-keratin extract, wherein the keratin composition has less than about 400 endotoxin units per composition.

Embodiment 52. A keratin composition comprising a beta-keratin network, wherein the keratin composition has less than about 400 endotoxin units per composition.

Embodiment 53. The keratin composition according to embodiment 51 or 52, wherein the keratin composition has less than about 20.0 endotoxin units per composition.

Embodiment 54. A keratin composition comprising a beta-keratin extract, wherein the keratin composition has less than about 400 endotoxin units per mg of the composition.

Embodiment 55. A keratin composition comprising a beta-keratin network, wherein the keratin composition has less than about 400 endotoxin units per mg of the composition.

Embodiment 56. The keratin composition according to embodiment 54 or 55, wherein the keratin composition has less than about 20.0 endotoxin units per mg of the composition.

Embodiment 57. A keratin composition comprising a beta-keratin extract, wherein the keratin composition has less than about 400 endotoxin units per cm².

Embodiment 58. A keratin composition comprising a beta-keratin network, wherein the keratin composition has less than about 400 endotoxin units per cm².

Embodiment 59. The keratin composition according to embodiment 57 or 58, wherein the keratin composition has less than about 20.0 endotoxin units per cm².

Embodiment 60. A keratin composition comprising a beta-keratin extract, wherein the keratin composition has less than about 400 endotoxin units per cm³.

Embodiment 61. A keratin composition comprising a beta-keratin network, wherein the keratin composition has less than about 400 endotoxin units per cm³.

Embodiment 62. The keratin composition according to embodiment 60 or 61, wherein the keratin composition has less than about 20.0 endotoxin units per cm³.

Embodiment 63. A keratin composition comprising a beta-keratin network, wherein the keratin composition has a moisture vapor transmission that is at least about 1000 g/(m²*day) of material per day.

Embodiment 64. The keratin composition according to any one of embodiments 52, 53, 55, 56, 58, 59 or 61-63, wherein the keratin composition has a moisture vapor transmission that is at least about 1000 g/(m²*day) of material per day.

Embodiment 65. A keratin composition comprising a beta-keratin network, wherein the keratin composition has a thickness of greater than about 0.2 mm.

Embodiment 66. The keratin composition according to any one of embodiments 52, 53, 55, 56, 58, 59 or 61-65, wherein the keratin composition has a thickness of greater than about 0.2 mm.

Embodiment 67. The keratin composition according to embodiment 65 or 66, wherein the keratin composition has a thickness ranging from about 0.2 mm to about 2 mm.

Embodiment 68. The keratin composition according to any one of embodiments 51-67, wherein the keratin composition comprises at least about 15 mg/cm³ of beta-keratin monomers, oligomers, and/or filaments.

Embodiment 69. The keratin composition according to any one of embodiments 52, 53, 55, 56, 58, 59 or 61-68, wherein the keratin composition is a continuous material.

Embodiment 70. The keratin composition according to embodiment 69, wherein the continuous material is in a form chosen from a sheet, a gel, a fiber and a foam.

Embodiment 71. The keratin composition according to embodiment 70, wherein the continuous material is a sheet that is fenestrated.

Embodiment 72. A keratin composition comprising a beta-keratin extract, wherein the keratin composition has less than about 400 endotoxin units per about 1 mL of the composition.

Embodiment 73. A keratin composition comprising a beta-keratin network, wherein the keratin composition has less than about 400 endotoxin units per about 1 mL of the composition.

Embodiment 74. The keratin composition according to embodiment 72 or 73, wherein the keratin composition has less than about 20 endotoxin units per about 1 mL of the composition.

Embodiment 75. The keratin composition according to any one of embodiments 51-56 or 72-74, wherein the keratin composition is in a form chosen from a dispersion and particles, such as a powder.

Embodiment 76. The keratin composition according to embodiment 75, wherein the dispersion is a suspension.

Embodiment 77. The keratin composition according to embodiment 75, wherein the keratin composition is in the form of a particle having an average particle size range of from about 8 nm to about 2 mm.

Embodiment 78. The keratin composition according to any one of embodiments 51-77, wherein the keratin composition is free of crosslinking agents.

Embodiment 79. The keratin composition according to any one of embodiments 51-78, wherein the keratin composition further comprises at least one additive.

Embodiment 80. The keratin composition according to embodiment 79, wherein the at least one additive is chosen from a protein, a carbohydrate, a polymer, a plasticizer, an antimicrobial agent, an anti-scarring agent, an anti-inflammatory agent, a pro-inflammatory agent, an angiogenic agent, a cell, a bulking agent, a stabilizer, a proteoglycan and a polysaccharide.

Embodiment 81. The keratin composition according to any one of embodiments 51-80, wherein the keratin composition comprises at least one plasticizer and at least one polymer.

Embodiment 82. The keratin composition according to embodiment 80 or 81, wherein the protein is chosen from an alpha-keratin monomer, oligomer and/or filament, a keratin-associated protein, a collagen, an elastin, a glycoprotein, a growth factor, a cytokine, silk, laminin, an antibody, an antimicrobial peptide, an integrin, an enzyme, an interleukin and a proteoglycan.

Embodiment 83. The keratin composition according to any one of embodiments 80-82, wherein the plasticizer is glycerol.

Embodiment 84. The keratin composition according to any one of embodiments 80-83, wherein the polymer is polyethylene glycol.

Embodiment 85. The keratin composition according to any one of embodiments 51-82 and 84, wherein the keratin composition does not comprise glycerol.

Embodiment 86. The keratin composition according to any one of embodiments 51-85, wherein the keratin composition further comprises alpha-keratin monomers, oligomers and/or filament.

Embodiment 87. The keratin composition according to any one of embodiments 51-86, wherein the keratin composition allows for cells to attach to the composition.

Embodiment 88. The keratin composition according to any one of embodiments 51-87, wherein the keratin composition allows for cell proliferation.

Embodiment 89. A method of preparing a keratin composition comprising combining a protein extract according to any one of embodiments 1-12 and 47-50 with at least one additive to give a keratin composition.

Embodiment 90. The method of embodiment 89, further comprising mixing the protein extract with a solvent prior to combining with the at least one additive.

Embodiment 91. The method of embodiment 90, wherein the solvent comprises water, a buffer, ethanol, methanol, poly(vinyl alcohol), formic acid, isopropanol, hexane and/or HFIP.

Embodiment 92. The method according to any one of embodiments 89-91, wherein the method further comprises casting the keratin composition onto a surface, such as a mold.

Embodiment 93. The method according to any one of embodiments 89-92, wherein the method further comprises freezing the keratin composition.

Embodiment 94. The method according to any one of embodiments 89-93, wherein the method further comprises dehydrating the keratin composition.

Embodiment 95. The method according to any one of embodiments 89-94, wherein the method further comprises cutting and/or milling the keratin composition.

Embodiment 96. The method according to any one of embodiments 89-91, wherein the method further comprises spinning the keratin composition into a keratin fiber.

Embodiment 97. The method of embodiment 96, wherein the method further comprises fabricating keratin fibers into a sheet.

Embodiment 98. The method according to any one of embodiments 89-97, wherein the method further comprises crosslinking the keratin composition.

Embodiment 99. The method according to any one of embodiments 89-98, wherein the crosslinking is chemical and/or physical crosslinking.

Embodiment 100. The method according to embodiment 98 or 99, wherein the crosslinking is a combination of chemical and physical crosslinking.

Embodiment 101. The method of embodiment 100, wherein the chemical crosslinking can be achieved through chemical reaction of complementary groups, crosslinking with high energy radiation, enzymatic crosslinking, free radical polymerization, photo crosslinking, crosslinking with chemical crosslinkers and/or UV crosslinking.

Embodiment 102. The method according to any one of embodiments 89-101, wherein the at least one additive is chosen from a protein, a carbohydrate, a polymer, a plasticizer, an antimicrobial agent, an anti-scarring agent, an anti-inflammatory agent, a pro-inflammatory agent, an angiogenic agent, a cell, a bulking agent, a stabilizer, a proteoglycan and a polysaccharide.

Embodiment 103. The method composition according to any one of embodiments 89-102, wherein the keratin composition comprises at least one plasticizer and at least one polymer.

Embodiment 104. The method according to embodiment 102 or 103, wherein the protein is chosen from an alpha-keratin monomer, oligomer and/or filament, a keratin-associated protein, a collagen, an elastin, a glycoprotein, a growth factor, a cytokine, silk, laminin, an antibody, an antimicrobial peptide, an integrin, an enzyme an interleukin and a proteoglycan.

Embodiment 105. The method according to any one of embodiments 102-104, wherein the plasticizer is glycerol.

Embodiment 106. The method according to any one of embodiments 102-105, wherein the polymer is polyethylene glycol.

Embodiment 107. The method according to any one of embodiments 89-104 and 106, wherein the keratin composition does not comprise glycerol.

Embodiment 108. A keratin composition prepared according to any one of embodiments 89-107.

Embodiment 109. A method of treating a wound of a subject comprising administering to the wound the keratin composition according to any one of embodiments 51-88 or 108, or a protein extract according to any one of embodiments 1-12 or 47-50.

Embodiment 110. The method according to embodiment 109, wherein the treating comprises healing the wound, closing the wound and/or reducing signs of scars.

Embodiment 111. A method of managing a wound of a subject comprising covering a wound, absorbing exudate from a wound, and/or maintaining appropriate moisture balance within a wound with the keratin composition according to any one of embodiments 51-88 or 108, or a protein extract according to any one of embodiments 1-12 or 47-50.

Embodiment 112. The method according to any one of embodiments 109-111, wherein the wound is an acute or chronic wound chosen from an abrasion, laceration, skin tear, tunneled wound, incision, draining wound, ulcer and/or burn.

Embodiment 113. The method according to any one of embodiments 109-112, wherein the wound is a wound of the dermis, soft tissue, connective tissue, eye, nerve, gum, dura, or bone.

Embodiment 114. A method for protecting a tissue of a subject comprising administering the keratin composition according to any one of embodiments 51-88 or 108, or a protein extract according to any one of embodiments 1-12 or 47-50 to the tissue.

Embodiment 115. The method according to embodiment 114, wherein the tissue is chosen from the dermis, soft tissue, connective tissue, eye, nerve, gum and bone.

Embodiment 116. The method according to any one of embodiments 109-115, wherein the keratin composition or protein extract is applied to the surface of the wound or the tissue.

Embodiment 117. The method according to any one of embodiments 109-116, wherein the protein extract is dehydrated.

Embodiment 118. A method for delivering a pharmaceutically active agent to a subject in need thereof, comprising administering to the subject the keratin composition according to any one of embodiments 51-88 or 108, wherein the keratin composition further comprises the pharmaceutically active agent.

Embodiment 119. The method according to embodiment 118, wherein the pharmaceutically active agent is chosen from a protein, an antimicrobial agent, an anti-scarring agent, an anti-inflammatory agent, a pro-inflammatory agent, a small molecule, an angiogenic factor, a gene therapy and a cell.

Embodiment 120. The method according to embodiment 119, wherein the protein is chosen from an alpha-keratin monomer, oligomer and/or filament, a keratin-associated protein, a collagen, an elastin, a glycoprotein, a growth factor, a cytokine, silk, laminin, an antibody, an antimicrobial peptide, an integrin, an enzyme, an interleukin and a proteoglycan.

Embodiment 121. A kit comprising the protein extract according to any one of embodiments 1-12 or 47-50.

Embodiment 122. A kit comprising the keratin composition according to any one of embodiments 51-88 or 108.

Embodiment 123. The kit according to embodiment 121 or 122, wherein the kit further comprises a syringe.

Embodiment 124. The kit according to any one of embodiments 121-123, wherein the kit further comprises sutures.

Embodiment 125. The kit according to any one of embodiments 121-124, wherein the kit further comprises a wound dressing, an adhesive material, a non-adhesive material, an antimicrobial agent, a debriding agent, or saline.

Embodiment 126. A method of cleaning a beta-keratin source material comprising:
a) combining a beta-keratin source material with a first solution comprising a first ionic detergent;
b) soaking and/or agitating the beta-keratin source material in the first solution;
c) rinsing the beta-keratin source material with water;
d) soaking and/or agitating the beta-keratin source material in a second solution comprising a second ionic detergent;
e) rinsing the beta-keratin source material with water, and
f) optionally sterilizing the beta-keratin source material.

Embodiment 127. The method according to embodiment 126, wherein the method further comprises a step of drying the beta-keratin source material before step (d).

Embodiment 128. The method according to embodiment 126 or 127, wherein the first ionic detergent and/or second ionic detergent comprises sodium dodecyl sulfate.

Embodiment 129. A method of cleaning a beta-keratin source material comprising:
a) combining a beta-keratin source material with a first solution comprising at least about 0.1% v/v of a polyethylene glycol-based detergent in water; and
b) soaking and/or agitating the beta-keratin source material in the first solution

Embodiment 130. The method according to embodiment 129, wherein the method further comprises a step (c) rinsing the beta-keratin source material with water, optionally drying the rinsed beta-keratin source material, and optionally sterilizing the rinsed or dried beta-keratin source material.

Embodiment 131. The method according to embodiment 130, wherein the method further comprises a step (d) soaking and/or agitating the beta-keratin source material in a second solution comprising a second detergent.

Embodiment 132. The method according to embodiment 131, wherein the method further comprises a step (e) rinsing the beta-keratin source material with water, optionally drying the rinsed beta-keratin source material, and optionally sterilizing the rinsed or dried beta-keratin source material.

Embodiment 133. The method according to embodiment 131 or 132, wherein the second detergent comprises a polyethylene glycol-based detergent.

Embodiment 134. A method of cleaning a beta-keratin source material comprising:
a) combining a beta-keratin source material with a first solution comprising a polyethylene glycol-based detergent;
b) soaking and/or agitating the beta-keratin source material in the first solution;
c) rinsing the beta-keratin source material with water and optionally drying the beta-keratin source material;
d) soaking and/or agitating the beta-keratin source material in a second solution comprising a polyethylene glycol-based detergent;
e) rinsing the beta-keratin source material with water; and
f) optionally sterilizing the beta-keratin source material.

Embodiment 135. The method according to any one of embodiments 129-134, wherein the polyethylene glycol-based detergent in step (a) comprises a polyethylene glycol tert-octylphenyl ether.

Embodiment 136. The method according to any one of embodiments 129-135, wherein the polyethylene glycol-based detergent in step (a) comprises a compound of formula (I):

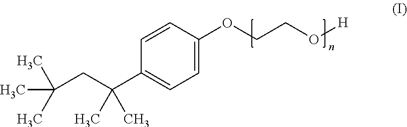

wherein n is chosen from 7-8, 9-10 or 40.

Embodiment 137. The method according to any one of embodiments 131-136, wherein the detergent in step (d) comprises a polyethylene glycol tert-octylphenyl ether.

Embodiment 138. The method according to any one of embodiments 131-137, wherein the detergent in step (d) comprises a compound of formula (I):

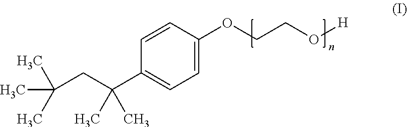

wherein n is chosen from 7-8, 9-10 or 40.

Embodiment 139. The method according to any one of embodiments 134-138, wherein the first solution in step (a) comprises at least about 0.1% v/v of a polyethylene glycol-based detergent in water.

Embodiment 140. The method according to any one of embodiments 131-139, wherein the second solution in step (d) comprises at least about 0.1% v/v of a polyethylene glycol-based detergent in water.

Embodiment 141. The method according to any one of embodiments 126-140, wherein the beta-keratin source material comprises an avian source material, for example goose feathers and/or chicken feathers, and/or a reptilian source material, for example alligator scales.

Embodiment 142. The method according to any one of embodiments 126-141, wherein the cleaned beta-keratin source material has less than about 3.25 endotoxin units per mg dry weight of cleaned beta-keratin source material.

Embodiment 143. The method according to any one of embodiments 126-142, wherein the cleaned beta-keratin source material has less than about 3.0 endotoxin units per mg dry weight of cleaned beta-keratin source material.

Embodiment 144. A cleaned beta-keratin source material prepared according to any one of embodiments 126-143.

These and other aspects and various embodiments are described more fully below.

### BRIEF DESCRIPTION OF THE DRAWINGS

FIG. 1A provides SDS-PAGE analysis of multiple keratin extracts (in extraction solution) from Extraction Processes 3 and 1a compared to a standard (“Marker”).

FIG. 1B provides SDS-PAGE analysis of a keratin extract (in extraction solution) from Extraction Process 4 compared to a standard (“Marker”).

FIG. 1C provides SDS-PAGE analysis of two keratin extracts (in extraction solution) from Extraction Process 2 compared to a standard (“Marker”).

FIG. 1D provides SDS-PAGE analysis of two keratin extracts (in extraction solution) from Extraction Process 1b compared to a standard (“Marker”).

FIG. 1E provides SDS-PAGE analysis of keratin extracts (in extraction solution) from Extraction Process 5 compared to a standard (“Marker”).

FIG. 1F provides SDS-PAGE analysis of keratin extracts (in extraction solution) from Extraction Processes 1a, 1b, 2, 3, 5, and 6 compared to a standard (“Marker”).

FIG. 2 provides examples of the solubility rating system discussed in Example 8.

FIG. 3 provides an example FTIR spectrum of a keratin sheet prepared using keratin extracts produced by Extraction Process 2. The arrow denotes the characteristic β-sheet signal.

FIG. 4 provides an image of WS1 fibroblast cells adhered to a beta-keratin extract as discussed in Example 10.

### DESCRIPTION OF THE EMBODIMENTS

### I. Definitions

As used herein, “a” or “an” means “at least one” or “one or more” unless specified otherwise. As used herein, the term “or” means “and/or” unless specified otherwise. In the context of a multiple dependent claim, the use of “or” when referring back to other claims refers to those claims in the alternative only.

As used herein, the term “about” refers to a numeric value, including, for example, whole numbers, fractions, and percentages, whether or not explicitly indicated. The term “about” generally refers to a range of numerical value (e.g., +/−5-10% of the recited range) that one of ordinary skill in the art would consider equivalent to the recited value (e.g., having the same function or result). When terms such as “at least,” “above,” and/or “about” precede a list of numerical values or ranges, the terms modify all of the values or ranges provided in the list. In some instances, the term “about” may include numerical values that are rounded to the nearest significant figure. When used in the content of thiol concentration at about 1% weight extract per volume using Ellman's assay, the term “about” means±0.20 mM. When used in the content of thiol concentration at about 0.1% weight extract per volume using Ellman's assay, the term “about” means±0.10 mM.

The units of measure listed herein should be understood to be convertible as one of skill in the art would understand. For example, 1 mL equals 1 cubic centimeter (“cc” or “cm³”).

As used herein, the term “keratin monomers” refers to one or more individual keratin protein molecules.

As used herein, the term “beta-keratin monomers” refers to one or more individual keratin protein molecules having molecular weight of from about 10 kDa to about 40 kDa. Beta-keratin monomers may be isolated from cornified epidermal appendages of reptiles and birds, such as claws, scales, beaks, and feathers. Birds include, for example, geese and chickens. Reptiles include, for example, lizards and alligators.

As used herein, the term “alpha-keratin monomers” refers to one or more individual keratin protein molecules having a molecular weight of from about 40 kDa to about 70 kDa. Alpha-keratin monomers may be isolated from hooves, horns, wool, hair and skin of vertebrates.

As used herein, the term “keratin oligomers” refers to one or more higher order molecular weight structures formed from interactions between keratin monomers. The term “keratin oligomers” includes but is not limited to, for example, keratin dimers and keratin tetramers.

As used herein, the term “keratin filaments” refers to one or more elongated aggregate of keratin oligomers. The term “keratin filaments” includes alpha-keratin intermediate filaments of about 7-10 nm diameter as well as beta-keratin filaments of about 3-4 nm diameter. Beta-keratin filaments are sometimes referred to as corneous beta-proteins (CBPs).

As used herein, the terms “protein extract” and “keratin extract” refer interchangeably to an extract comprising keratin monomers and/or oligomers that has been isolated from a keratin source such as wool, hair, skin, claws, scales, beaks and feathers. A protein extract can be in liquid form (for example, when the monomers and/or oligomers are in an extract solution), semi-solid (for example, a gel) or solid form (for example, when an extract solution containing monomers and/or oligomers is dehydrated). The term “beta-keratin extract” refers to beta-keratin monomers and/or oligomers that have been isolated from claws, scales, beaks and/or feathers.

As used herein, a keratin “source” refers to any material that contains keratin, including keratin monomers, oligomers, filaments and/or networks. Alpha-keratin source materials include wool, hair and skin of vertebrates. Beta-keratin source materials are cornified epidermal appendages of reptiles and birds (e.g., claws, scales, beaks and feathers).

As used herein, “cleaning” (and other forms of the term) means to remove contaminates such as debris, dirt, bioburden, endotoxins, and other unwanted substances. In some embodiments, contaminates include, but are not limited to, non-keratin based tissues, DNA, non-keratin proteins, other extracellular matrix components, microorganisms, lipids, inorganic matter, other organic matter, pigments, and/or cells. In some embodiments, cleaning removes endotoxins. In some embodiments, cleaning removes all contaminates. In some embodiments, cleaning removes a portion of the contaminates. Cleaning can be accomplished by various means, including those described herein.

As used herein, “agitate” (and other forms of the term) means to stir, mix or disturb a material, such as a keratin source, in a medium. Agitating can be accomplished by known methods including, for example, scrubbing or stirring. Agitation can be achieved by hand or by mechanical means. For example, agitation can be achieved by shaking by hand or mixing with a stir bar, a vortex, a sonicator, paint mixer, washing machine, etc. Agitating can be for any appropriate duration.

As used herein, the terms “soak” or “soaking” refer to allowing a material, such as a keratin source, to sit in a medium for a period of time. The medium can be chosen from, for example, water, a detergent solution, a salt solution, a buffer solution, or organic or inorganic solvent. Soaking does not require agitation of the material and the medium.

As used herein, the term “wash” or “washing” refers to agitating or soaking a material, such as a keratin source, in a medium. The medium can be chosen from, for example, water, a detergent solution, a salt solution, a buffer solution, or organic or inorganic solvent.

As used herein, the term “defatting” refers to a process used to remove lipids from a keratin source.

As used herein, the term “detergent” refers to a surfactant or mixture of surfactants. Detergents can be an ionic detergent, zwitterionic detergent or a non-ionic detergent. Ionic detergents include both anionic and cationic detergents. Anionic detergents include, for example, phosphorylated and sulfonated straight-chain hydrocarbons such as sodium dodecyl sulfate (SDS). Non-ionic detergents include, for example, polyethylene glycol-based detergents and N-methylglucamide-based detergents. Zwitterionic detergents include, for example, N,N-Bis[3-(D-gluconamido)propyl]cholamide-based detergents.

As used herein, the term “sterilization” and the like in the context of cleaning a keratin source material refers to a process used to inactivate, remove, degrade, destroy, kill, and/or deactivate a contaminate such as microorganisms and endotoxins. In some embodiments, sterilization degrades endotoxins from a keratin source material. In some embodiments, sterilization inactivates or kills a microorganism, such as bacteria, from a keratin source material. In some embodiments, sterilization results in undetectable and/or non-viable microorganisms. In the context of keratin extracts or compositions, “sterile” refers to extracts or compositions that are essentially free from viable microorganisms, such as bacteria and fungi. In some embodiments, a “sterile” composition is a composition with a sterility assurance level (SAL) of equal to or less than about 10-3, such equal to or less than about 10-6. In some embodiments, sterilization of a keratin source material or a keratin composition can occur by heating. In some embodiments, sterilization of a keratin source material or a keratin composition can occur by autoclaving. In some embodiments, sterilization of a keratin source material or a keratin composition can occur by electron beam radiation. In some embodiments, sterilization of a keratin source material or a keratin composition can occur by gamma radiation.

As used herein, the term “dehydrated” and the like refer to a sample in which at least some of the water has been removed. For example, a dehydrated protein extract is a protein extract wherein at least some of the water has been removed. In some embodiments, all of the water has been removed from the sample. Dehydration can be accomplished by known methods including heating, air drying, drying at a specific humidity and lyophilization.

As used herein, the term “soluble” when referring to a keratin extract means the keratin extract forms a solution with no visible material within about 5 minutes of being combined with a solvent. A soluble keratin extract is generally homogenous and optionally tinted.

As used herein, the term “slightly soluble” when referring to a keratin extract means the keratin extract forms a cloudy, disperse solution within about 5 minutes of being combined with a solvent.

As used herein, the term “significantly insoluble” when referring to a keratin extract means a majority of the keratin extract remains in solid state, suspended in solution within about 5 minutes of being combined with a solvent.

As used herein, the term “insoluble” when referring to a keratin extract means that an insignificant amount of the keratin extract goes into solution within about 5 minutes of being combined with a solvent.

As used herein, “cysteine modified keratin” and “cysteine modified keratin protein” refer to keratin monomers, oligomers, and/or filaments wherein at least some of the cysteine residues have been modified. Modifications include, for example, oxidation or alkylation of cysteine. Oxidized keratin protein can, for example, be prepared by using an oxidant, such as performic acid. Alkylated keratin include, for example, partially carboxylated keratin monomers and/or oligomers or partially carboxymethylated keratin monomers and/or oligomers.

As used herein, the terms “reduced keratin” and “reduced keratin protein” refer to keratin monomers and/or oligomers wherein at least some of the cystine residues of a keratin protein have been converted to cysteine-S-sulfonates. Reduced keratins can be prepared by using reducing agents, such as thioglycolic acid, sodium thioglycolate, potassium cyanide and sodium sulfide.

As used herein, the term “room temperature” refers to a range of air temperatures that are typical for indoor settings. Room temperature generally ranges from about 20° C. to about 25° C. In some situations, room temperature ranges from about 21° C. to about 22° C.

As used herein, the term “network” refers to a 3-dimensional structure of proteins that results from crosslinking of the protein molecules with or without additives. In some embodiments, the network can comprise additional components, such as proteins, carbohydrates, polymers, plasticizers, antimicrobial agents, anti-scarring agents, anti-inflammatory agents, cells, bulking agents, stabilizers and polysaccharides. In some embodiments, the network is formed by the creation of disulfide bonds between keratin monomers and/or oligomers.

As used herein, the terms “crosslink,” “crosslinked,” “crosslinking” and the like refer to interactions taking place between keratin monomers, oligomers and/or additives. Crosslinks may be physical, chemical or a combination thereof. Physical crosslinks include hydrophobic or hydrophilic bonds, bonds through polarity or Van der Waals forces, aggregation or coacervation effects, and ionic binding or ionic complex formation. Chemical crosslinks include covalent bonds, such as disulfide bonds. Crosslinking may occur spontaneously or may be induced. For example, crosslinking can be achieved and/or facilitated using chemical reactions with complementary groups, high energy radiation, enzymes, free radicals, photo crossing, chemical crosslinkers, and UV light. Chemical crosslinkers include, for example, formic acid and glutaraldehyde.

As used herein, the term “continuous material” refers to a material with an uninterrupted network, such as protein network, that is a solid or semi-solid at room temperature. Continuous materials include, by are not limited to, sheets, gels, foams, and fibers.

As used herein, the terms “sheet” and “film” are interchangeable and refer to a continuous polymeric material. Sheets can be of any shape, size, and thickness. Sheets include coatings on the surface of another material or device. Sheets also include nanofibrous mats, which are fibrous nanomaterials often fabricated through electrospinning.

As used herein, the term “hydrogel” refers to a three-dimensional network of proteins that are crosslinked and can absorb and/or retain water or other fluids. Hydrogels may be formed by physical or chemical crosslinking, electrospinning, 3D printing, salt or porogen templating, thermogelation and/or cryogelation. Hydrogels may absorb and/or retain a large amount of a fluid, for example, greater than about 40% of water. Hydrogels may contain additional components including, for example, a carbohydrate, a polymer or glycerol.

As used herein, the term “gel” refers to a semi-solid material and encompasses hydrogels, cryogels and thermogels.

As used herein, the term “particle” refers to a granular material composed of one or more materials, such as keratin. In some embodiments, the average size of the granules is from about 1 nm to about 10 mm.

As used herein, the term “powder” refers to particles that are a solid or a mixture of solids that have been reduced to a finely divided state. Powders can be composed of one or more materials, such as keratin monomers or filaments. For example, in one embodiment, the powder comprises keratin and an additive such as a buffer, salt, surfactant, di- or polysaccharide, amino acid, and antioxidant.

As used herein, the term “dispersion” refers to a mixture in which particles of one substance are scattered through a continuous phase of another substance. Dispersions can be heterogenous or homogenous mixtures. Dispersions include, among other things, slurries, suspensions, ointments, creams, colloids and sprayable dispersions. As used herein, a “slurry” refers to a mixture of particles in a continuous phase of another substance where the particles are not entirely suspended. As used herein, a “suspension” refers to a heterogeneous mixture in which the solute particles are suspended throughout the continuous phase of the other substance. As used herein, a “cream” refers to an emulsion where the particles are suspended throughout. As used herein, the term “colloid” refers to a mixture in which microscopically dispersed particles are suspended throughout another substance. As used herein, the term “sprayable dispersion” refers to a dispersion that is broken up into minute droplets and blown, ejected into, or falling through the air.

Dispersions can be prepared by, for example, mixing solid particles of a substance with a liquid or gel. A keratin dispersion can be keratin particles scattered through a continuous phase of another substance, or it can be particles of another substance scattered through a continuous phase of a keratin network.

As used herein, the term “keratin sprayable liquid” refers to a solution containing beta-keratin dissolved in a solvent that is broken up into minute droplets and blown, ejected into, or falling through the air.

As used herein, the terms “foam” and “sponge” are interchangeable and refer to a porous polymer scaffold. A keratin foam refers to a foam wherein the polymer scaffold is principally made of a keratin network. Foams can include additional components such as, but not limited to, polymers and polyols. Foams can be prepared by, for example, salt or porogen templating and lyophilization. Foams can be sprayable or in the form of a sheet.

As used herein, the term “fiber” refers to a natural or synthetic structure with an outer diameter less than about 1000 nm and is longer than it is wide. A protein fiber is a fiber composed of a protein network, optionally including additional components such as, for example, other polymers or an adhesive. Fibers can be prepared by, for example, wet spinning, electrospinning, or 3D printing. Fibers can be used as they are or fabricated into nanofibrous mats, for example.

As used herein, the term “casting” refers to a process of preparing a polymeric continuous material, which may or may not include additional components, by dispensing a solution or dispersion of the polymer and any other components onto a surface, followed by dehydration. The dispensing may be achieved by pouring or printing. A polymer network may form before, during or after the dehydration step. The surface can include a tray, mold or other device. The polymer may be keratin, such as beta-keratin. The additional components may include, for example, other polymers, carbohydrates, small molecules, or other proteins.

As used herein, the terms “milling” or “homogenizing” refer to grinding of a sample. Milling can be accomplished by, for example, a mortar and pestle, cryo-mill, blender or a knife mill. In some embodiments, the sample can be a keratin composition that is a continuous material. In some embodiments, the sample is a keratin composition in the form of a sheet.

As used herein, the term “treating” refers to an approach for obtaining certain beneficial or desired clinical results. In some embodiments, “treating” refers to healing of a wound.

As used herein, the term “wound management” refers to optimizing a wound environment by covering a wound, absorbing exudate from a wound, and/or maintaining appropriate moisture balance within a wound.

As used herein, the term “protecting” refers to an attempt to keep safe from harm or attempt to keep safe from further injury. The injury can be, for example, a wound. Protection may be accomplished, for example, by covering a living tissue and/or a wound.

As used herein, the term “wound” refers to a breakdown in the protective function of a living tissue, including but not limited to a breakdown of the dermis, soft tissue, connective tissue, eye, nerve, gums, dura, or bones. Wounds can be acute or chronic. In some embodiments, the wound is an abrasion, laceration, skin tear, tunneled wound, incision, burn, draining wound and/or ulcer. In some embodiments, the wound is a surgical wound including an acute surgical wound, donor site or graft, podiatric wound, post-Moh's surgery, post-laser surgery, and/or wound dehiscence. In some embodiments, the burn is a first-, second- or third-degree burn. In some embodiments, the burn is a partial thickness burn. In some embodiments, the burn is chosen from a friction burn, cold burn, thermal burn, radiation burn, chemical burn and electrical burn. In some embodiments, the radiation burn is a sunburn. In some embodiments, the ulcer is chosen from a pressure ulcer, a venous ulcer, a diabetic ulcer, an ulcer caused by mixed vascular etiologies and a chronic vascular ulcer. In some embodiments, the wound is a partial or full thickness wound.

As used herein, the term “wound dressing” refers to a material or product intended to clean, cover, and/or protect a wound from the external environment.

As used herein, the term “cell attachment” refers to a process by which cells interact with and/or attach to a keratin composition. Cell attachment can be shown by immunohistochemistry and imaging or histological analysis.

As used herein, the term “cell proliferation” refers to an increase in the number of cells. Cell proliferation can be shown, for example, by using a Bromodeoxyuridine (BrdU) assay and/or a WS1 human fibroblast assay such as described below in Example 10, and/or other methods known in the art. See e.g., Matatall et al. Methods Mol Biol. 2018; 1686:91-103. doi: 10.1007/978-1-4939-7371-2_7.

### II. Exemplary Embodiments of Methods of Cleaning a Keratin Source Material and Cleaned Keratin Source Materials

Keratin source materials contain unwanted impurities, such as bioburden, endotoxin, and environmental contaminants. Impurities in the keratin source material may negatively affect the final keratin containing products and thus it is desirable to remove any such impurities prior to preparation of keratin extracts and compositions.

Thus, in one aspect, methods of cleaning a keratin source material, and the cleaned products thereof, are disclosed herein. In some embodiments, the keratin source material comprises a beta-keratin source material. In some embodiments, the beta-keratin source material comprises epidermal appendages of reptiles and birds.

In some embodiments, the beta-keratin source material comprises epidermal appendages of reptiles. In some embodiments, the beta-keratin source material comprises epidermal appendages of alligators. In some embodiments, the beta-keratin source material comprises alligator scales.

In some embodiments, the beta-keratin source material comprises epidermal appendages of birds. In some embodiments, the beta-keratin source material comprises an avian source material. In some embodiments, the beta-keratin source material comprises feathers. In some embodiments, the beta-keratin source material comprises goose and/or chicken feathers. In some embodiments, the beta-keratin source material comprises white chicken feathers. In some embodiments, the beta-keratin source material comprises brown chicken feathers. In some embodiments, the beta-keratin source material comprises black chicken feathers.

In some embodiments, the method of cleaning uses a detergent. In some embodiments, the detergent comprises an ionic detergent, zwitterionic detergent and/or a non-ionic detergent. In some embodiments, the ionic detergent comprises a cationic and/or anionic detergent.

In some embodiments, the method of cleaning comprises sterilization of the source material.

In some embodiments, the method of cleaning reduces the endotoxin levels of a keratin source material. In some embodiments, the method of cleaning reduces the endotoxin levels of a keratin source material by at least about 14 percent compared to the uncleaned keratin source material. In some embodiments, the method of cleaning reduces the endotoxin levels of a keratin source material by at least about 15 percent compared to the uncleaned keratin source material. In some embodiments, the method of cleaning reduces the endotoxin levels of a keratin source material by at least about 20 percent compared to the uncleaned keratin source material. In some embodiments, the method of cleaning reduces the endotoxin levels of a keratin source material by at least about 30 percent compared to the uncleaned keratin source material. In some embodiments, the method of cleaning reduces the endotoxin levels of a keratin source material by at least about 40 percent compared to the uncleaned keratin source material. In some embodiments, the method of cleaning reduces the endotoxin levels of a keratin source material by at least about 50 percent compared to the uncleaned keratin source material.

In some embodiments, the method of cleaning reduces the endotoxin levels of a keratin source material by at least about 70 percent compared to the uncleaned keratin source material. In some embodiments the method of cleaning reduces the endotoxin levels of a keratin source material by at least about 99 percent compared to the uncleaned keratin source material. In some embodiments, the method of cleaning reduces the endotoxin levels of a keratin source material by at least about 99.9 percent compared to the uncleaned keratin source material.

In some embodiments, the method results in a cleaned beta-keratin source material having less than about 3.25 endotoxin units per mg of dry weight cleaned beta-keratin source material. In some embodiments, the method results in a cleaned beta-keratin source material having less than about 3.0 endotoxin units per mg dry weight of cleaned beta-keratin source material, less than about 2.5 endotoxin units per mg dry weight of cleaned beta-keratin source material, less than about 2.0 endotoxin units per mg dry weight of cleaned beta-keratin source material, less than about 1.5 endotoxin units per mg dry weight of cleaned beta-keratin source material, less than about 1.0 endotoxin units per mg dry weight of cleaned beta-keratin source material or less than about 0.5 endotoxin units per mg dry weight of cleaned beta-keratin source material.

In some embodiments, the method results in a cleaned beta-keratin source material having less than about 0.20 endotoxin units per mg dry weight of cleaned beta-keratin source material. In some embodiments, the method results in a cleaned beta-keratin source material having less than about 0.15 endotoxin units per mg dry weight of cleaned beta-keratin source material, less than about 0.10 endotoxin units per mg dry weight of cleaned beta-keratin source material, less than about 0.05 endotoxin units per mg dry weight of cleaned beta-keratin source material, less than about 0.04 endotoxin units per mg dry weight of cleaned beta-keratin source material, less than about 0.03 endotoxin units per mg dry weight of cleaned beta-keratin source material, less than about 0.02 endotoxin units per mg dry weight of cleaned beta-keratin source material or less than about 0.01 endotoxin units per mg dry weight of cleaned beta-keratin source material.

In some embodiments, the method results in a cleaned beta-keratin source material having less endotoxin units per mg dry weight of cleaned beta-keratin source material than is the detectable limits of the testing method. Testing can be performed using, for example, a kinetic chromogenic LAL assay as discussed in Example 2.

In some embodiments, the cleaned beta-keratin source material has from about 0.00004 to about 3.25 endotoxin units per mg dry weight of cleaned beta-keratin source material. In some embodiments, the cleaned beta-keratin source material has from about 0.00004 to about 3.0 endotoxin units per mg dry weight of cleaned beta-keratin source material. In some embodiments, the cleaned beta-keratin source material has from about 0.00004 to about 2.8 endotoxin units per mg dry weight of cleaned beta-keratin source material, from about 0.00004 to about 2.6 endotoxin units per mg dry weight of cleaned beta-keratin source material, from about 0.00004 to about 2.4 endotoxin units per mg dry weight of cleaned beta-keratin source material, from about 0.00004 to about 2.2 endotoxin units per mg dry weight of cleaned beta-keratin source material, from about 0.00004 to about 2.0 endotoxin units per mg dry weight of cleaned beta-keratin source material, from about 0.00004 to about 1.8 endotoxin units per mg dry weight of cleaned beta-keratin source material, from about 0.00004 to about 1.6 endotoxin units per mg dry weight of cleaned beta-keratin source material, from about 0.00004 to about 1.4 endotoxin units per mg dry weight of cleaned beta-keratin source material, from about 0.00004 to about 1.2 endotoxin units per mg dry weight of cleaned beta-keratin source material, and/or from about 0.00004 to about 1.0 endotoxin units per mg dry weight of cleaned beta-keratin source material.

In some embodiments, the cleaned beta-keratin source material has from about 0.00004 to about 0.90 endotoxin units per mg dry weight of cleaned beta-keratin source material, from about 0.00004 to about 0.80 endotoxin units per mg dry weight of cleaned beta-keratin source material, from about 0.00004 to about 0.70 endotoxin units per mg dry weight of cleaned beta-keratin source material, from about 0.00004 to about 0.60 endotoxin units per mg dry weight of cleaned beta-keratin source material, from about 0.00004 to about 0.50 endotoxin units per mg dry weight of cleaned beta-keratin source material, from about 0.00004 to about 0.40 endotoxin units per mg dry weight of cleaned beta-keratin source material, from about 0.00004 to about 0.30 endotoxin units per mg dry weight of cleaned beta-keratin source material, from about 0.00004 to about 0.20 endotoxin units per mg dry weight of cleaned beta-keratin source material, and/or from about 0.00004 to about 0.01 endotoxin units per mg dry weight of cleaned beta-keratin source material.

In some embodiments, the cleaned beta-keratin source material has from about 0.01 to about 0.10 endotoxin units per mg dry weight of cleaned beta-keratin source material, from about 0.01 to about 0.09 endotoxin units per mg dry weight of cleaned beta-keratin source material, from about 0.01 to about 0.08 endotoxin units per mg dry weight of cleaned beta-keratin source material, from about 0.01 to about 0.07 endotoxin units per mg dry weight of cleaned beta-keratin source material, from about 0.01 to about 0.06 endotoxin units per mg dry weight of cleaned beta-keratin source material, from about 0.01 to about 0.05 endotoxin units per mg dry weight of cleaned beta-keratin source material, from about 0.01 to about 0.04 endotoxin units per mg dry weight of cleaned beta-keratin source material, from about 0.01 to about 0.03 endotoxin units per mg dry weight of cleaned beta-keratin source material, and/or from about 0.01 to about 0.02 endotoxin units per mg dry weight of cleaned beta-keratin source material.

### A. Exemplary Embodiments Using Ionic Detergents

In some embodiments, the method of cleaning a beta-keratin source material comprises: (a) combining a beta-keratin source material with a first solution comprising a first ionic detergent; (b) soaking and/or agitating the beta-keratin source material in the first solution; (c) rinsing the beta-keratin source material with water; (d) soaking and/or agitating the beta-keratin source material in a second solution comprising a second ionic detergent; and (e) rinsing the beta-keratin source material with water.

In some embodiments, the beta-keratin source material is soaked in step (b) and/or (d). In some embodiments, the beta-keratin source material is agitated in step (b) and/or (d). In some embodiments, the beta-keratin source material is first soaked and then agitated in step (b) and/or (d). In some embodiments, the beta-keratin source material is first agitated and then soaked in step (b) and/or (d).

In some embodiments, the rinsing in steps (c) and/or (e) is accomplished by spraying water over the source material or agitating source material in water followed by draining.

In some embodiments, the method further comprises drying the beta-keratin source material before step (d). In some embodiments, the method further comprises drying the beta-keratin source material after step (e). In some embodiments, drying is accomplished by allowing the source material to dry at room temperature. In some embodiments, drying is accomplished by heating the rinsed source material from step (c) and/or (e) in an oven, air drying, or drying in a dryer.

In some embodiments, in any of the preceding methods, the first solution in step (a) comprises from about 0.1% v/v to about 1% v/v of a first ionic detergent in water. In some embodiments, in any of the preceding methods, the second solution in step (d) comprises from about 0.1% v/v to about 1% v/v of a second ionic detergent in water.

In some embodiments, the first solution in step (a) comprises about 0.5% v/v of a first ion detergent in water. In some embodiments, in any of the preceding methods, the second solution in step (d) comprises about 0.5% v/v of a second ionic detergent in water.

In some embodiments, the first ionic detergent and/or second ionic detergent comprises an anionic detergent. In some embodiments, the first ionic detergent and/or second ionic detergent comprises sodium n-dodecyl benzene sulfonate (“LAS”) and/or sodium dodecyl sulfate (“SDS”). In some embodiments, the first ionic detergent and/or second ionic detergent comprises SDS. In some embodiments, the first ionic detergent and second ionic detergent comprise SDS.

In some embodiments, the first solution and/or second solution further comprises a non-ionic detergent and/or zwitterionic detergent. In some embodiments, the non-ionic detergent comprises a polyethylene glycol-based detergent and/or a N-methylglucamide-based detergent. In some embodiments, the zwitterionic detergent comprises a N,N-Bis[3-(D-gluconamido)propyl]cholamide-based detergent.

In some embodiments, the keratin source material comprises a beta-keratin source material. In some embodiments, the beta-keratin source material comprises epidermal appendages of reptiles and/or birds. In some embodiments, the beta-keratin source material comprises epidermal appendages of reptiles. In some embodiments, the beta-keratin source material comprises epidermal appendages of alligators. In some embodiments, the beta-keratin source material comprises alligator scales. In some embodiments, the beta-keratin source material comprises epidermal appendages of birds. In some embodiments, the beta-keratin source material comprises an avian source material. In some embodiments, the beta-keratin source material comprises feathers. In some embodiments, the beta-keratin source material comprises goose and/or chicken feathers. In some embodiments, the beta-keratin source material comprises white chicken feathers. In some embodiments, the beta-keratin source material comprises brown chicken feathers. In some embodiments, the beta-keratin source material comprises black chicken feathers.

### B. Exemplary Embodiments Using Non-Ionic Detergents

In some embodiments, the method of cleaning a beta-keratin source material comprises: (a) combining a beta-keratin source material with a first solution comprising at least about 0.1% v/v of a polyethylene glycol-based detergent in water; and (b) soaking and/or agitating the beta-keratin source material in the first solution.

In some embodiments, the method further comprises a step (c) rinsing the beta-keratin source material with water and optionally drying the rinsed beta-keratin source material. In some embodiments, rinsing is accomplished by spraying water over the source material or agitating source material in water followed by draining. In some embodiments, drying is accomplished by allowing the source material to dry at room temperature. In some embodiments, drying is accomplished by heating the rinsed source material in an oven, air drying, or drying in a dryer.

In some embodiments, the method further comprises a step (d) soaking and/or agitating the beta-keratin source material in a second solution comprising a second detergent. In some embodiments, the beta-keratin source material is soaked in step (b) and/or (d). In some embodiments, the beta-keratin source material is agitated in step (b) and/or (d). In some embodiments, the beta-keratin source material is first soaked and then agitated in step (b) and/or (d). In some embodiments, the beta-keratin source material is first agitated and then soaked in step (b) and/or (d).

In some embodiments, the second detergent in step (d) comprises an ionic detergent, zwitterionic detergent and/or a non-ionic detergent. In some embodiments, the second detergent in step (d) comprises a non-ionic detergent. In some embodiments, the second detergent in step (d) comprises a polyethylene glycol-based detergent.

In some embodiments, the method further comprises a step (e) rinsing the beta-keratin source material with water. In some embodiments, rinsing is accomplished by spraying water over the source material or agitating source material in water followed by draining.

In some embodiments, the method of cleaning a beta-keratin source material comprises: (a) combining a beta-keratin source material with a first solution comprising a polyethylene glycol-based detergent; (b) soaking or agitating the beta-keratin source material in the first solution; (c) rinsing the beta-keratin source material with water; (d) soaking or agitating the beta-keratin source material in a second solution comprising a polyethylene glycol-based detergent; and (e) rinsing the beta-keratin source material with water.

In some embodiments, the beta-keratin source material is soaked in step (b) and/or (d). In some embodiments, the beta-keratin source material is agitated in step (b) and/or (d). In some embodiments, the beta-keratin source material is first soaked and then agitated in step (b) and/or (d). In some embodiments, the beta-keratin source material is first agitated and then soaked in step (b) and/or (d).

In some embodiments, the rinsing in steps (c) and/or (e) is accomplished by spraying water over the source material or agitating source material in water followed by draining.

In some embodiments, the method further comprises drying the beta-keratin source material after step (c). In some embodiments, drying is accomplished by allowing the source material to dry at room temperature. In some embodiments, drying is accomplished by heating the rinsed source material in an oven, air drying, or drying in a dryer.

In some embodiments, in any of the preceding methods, the polyethylene glycol-based detergent in step (a) comprises a polyethylene glycol tert-octylphenyl ether. In some embodiments, the polyethylene glycol-based detergent in step (a) comprises a compound of formula (I):

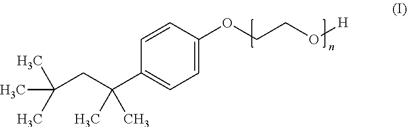

wherein n is chosen from 7-8, 9-10 or 40.

In some embodiments, in any of the preceding methods, the detergent in step (d) comprises a polyethylene glycol tert-octylphenyl ether. In some embodiments, the polyethylene glycol-based detergent in step (d) comprises a compound of formula (I):

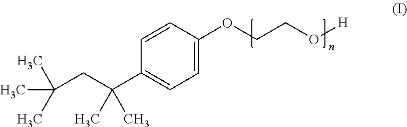

wherein n is chosen from 7-8, 9-10 or 40.

In some embodiments, the first solution in step (a) comprises at least about 0.1% v/v of a polyethylene glycol-based detergent in water. In some embodiments, in any of the preceding methods, the second solution in step (d) comprises at least about 0.1% v/v of a polyethylene glycol-based detergent in water.

In some embodiments, in any of the preceding methods, the first solution in step (a) comprises from about 0.1% v/v to about 10% v/v of a polyethylene glycol-based detergent in water. In some embodiments, in any of the preceding methods, the first solution in step (a) comprises from about 0.1% v/v to about 9% v/v, 0.1% v/v to about 8% v/v, 0.1% v/v to about 7% v/v, 0.1% v/v to about 6% v/v, 0.1% v/v to about 5% v/v, 0.1% v/v to about 4% v/v, 0.1% v/v to about 3% v/v, 0.1% v/v to about 2% v/v, and/or 0.1% v/v to about 1% v/v of a polyethylene glycol-based detergent in water.

In some embodiments, in any of the preceding methods, the second solution in step (d) comprises from about 0.1% v/v to about 10% v/v of a polyethylene glycol-based detergent in water. In some embodiments, in any of the preceding methods, the second solution in step (d) comprises from about 0.1% v/v to about 9% v/v, 0.1% v/v to about 8% v/v, 0.1% v/v to about 7% v/v, 0.1% v/v to about 6% v/v, 0.1% v/v to about 5% v/v, 0.1% v/v to about 4% v/v, 0.1% v/v to about 3% v/v, 0.1% v/v to about 2% v/v, and/or 0.1% v/v to about 1% v/v of a polyethylene glycol-based detergent in water.

In some embodiments, the method further comprises drying the cleaned beta-keratin source material after step (e) in any of the above embodiments. In some embodiments, drying is accomplished by allowing the source material to dry at room temperature. In some embodiments, drying is accomplished by heating the rinsed source material in an oven, air drying, or drying in a dryer.

In some embodiments, the keratin source material comprises a beta-keratin source material. In some embodiments, the beta-keratin source material comprises epidermal appendages of reptiles and/or birds. In some embodiments, the beta-keratin source material comprises epidermal appendages of reptiles. In some embodiments, the beta-keratin source material comprises epidermal appendages of alligators. In some embodiments, the beta-keratin source material comprises alligator scales. In some embodiments, the beta-keratin source material comprises epidermal appendages of birds. In some embodiments, the beta-keratin source material comprises an avian source material. In some embodiments, the beta-keratin source material comprises feathers. In some embodiments, the beta-keratin source material comprises goose and/or chicken feathers. In some embodiments, the beta-keratin source material comprises white chicken feathers. In some embodiments, the beta-keratin source material comprises brown chicken feathers. In some embodiments, the beta-keratin source material comprises black chicken feathers.

### C. Exemplary Embodiments Comprising Sterilization

In some embodiments, the methods of cleaning a beta-keratin source material as described herein further comprise a step of sterilization. In some embodiments, the sterilization occurs before the beta-keratin source material is combined with a detergent solution. In some embodiments, the sterilization occurs after rinsing the beta-keratin source material. In some embodiments, the sterilization occurs after drying the rinsed beta-keratin source material.

In some embodiments, the sterilization comprises heating the beta-keratin source material. In some embodiments, the sterilization comprises exposing the beta-keratin source material to heat. In some embodiments, the sterilization comprises exposing the beta-keratin source material to a temperature of about 100° C. to about 300° C. In some embodiments, the temperature is from about 150° C. to about 250° C., such as for example from about 175° C. to 250° C. or from about 180° C. to about 250° C.

In some embodiments, the sterilization comprises exposing the beta-keratin source material to a temperature of from about 100° C. to about 300° C. for a period of time. In some embodiments, the temperature is from about 150° C. to about 250° C., such as for example from about 175° C. to 250° C. or from about 180° C. to about 250° C. In some embodiments, the period of time is about 15 minutes. In some embodiments, the period of time is about 30 minutes. In some embodiments, the period of time is about 1 hour. In some embodiments, the period of time is about 75 minutes, about 2 hours, about 2.5 hours, about 3 hours, about 3.5 hours, about 4 hours, about 4.5 hours, about 5 hours, about 5.5 hours, about 6 hours, or about 6.5 hours.

In some embodiments, the sterilization includes wetting the beta-keratin source material prior to exposing the beta-keratin source material to heat. In some embodiments, the sterilization includes the use of steam.

In some embodiments, the sterilization occurs once. In some embodiments, the sterilization is repeated such as, for example, once, twice or three times.

### D. Exemplary Embodiments of Cleaned Keratin Source Materials

In some embodiments, a cleaned beta-keratin source material is prepared according to any of the embodiments disclosed above. In some embodiments, the beta-keratin source material comprises epidermal appendages of reptiles and/or birds.

In some embodiments, the beta-keratin source material comprises epidermal appendages of reptiles. In some embodiments, the beta-keratin source material comprises epidermal appendages of alligators. In some embodiments, the beta-keratin source material comprises alligator scales.

In some embodiments, the beta-keratin source material comprises epidermal appendages of birds. In some embodiments, the beta-keratin source material comprises an avian source material. In some embodiments, the beta-keratin source material comprises feathers. In some embodiments, the beta-keratin source material comprises goose and/or chicken feathers. In some embodiments, the beta-keratin source material comprises white chicken feathers. In some embodiments, the beta-keratin source material comprises brown chicken feathers. In some embodiments, the beta-keratin source material comprises black chicken feathers.

In some embodiments, the cleaned beta-keratin source material has less than about 3.25 endotoxin units per mg dry weight of cleaned beta-keratin source material. In some embodiments, the cleaned beta-keratin source material has less than about 3.0 endotoxin units per mg dry weight of cleaned beta-keratin source material, less than about 2.5 endotoxin units per mg dry weight of cleaned beta-keratin source material, less than about 2.0 endotoxin units per mg dry weight of cleaned beta-keratin source material, less than about 1.5 endotoxin units per mg dry weight of cleaned beta-keratin source material, less than about 1.0 endotoxin units per mg dry weight of cleaned beta-keratin source material or less than about 0.5 endotoxin units per mg dry weight of cleaned beta-keratin source material.

In some embodiments, the method results in a cleaned beta-keratin source material having less than about 0.20 endotoxin units per mg dry weight of cleaned beta-keratin source material. In some embodiments, the method results in a cleaned beta-keratin source material having less than about 0.15 endotoxin units per mg dry weight of cleaned beta-keratin source material, less than about 0.10 endotoxin units per mg dry weight of cleaned beta-keratin source material, less than about 0.05 endotoxin units per mg dry weight of cleaned beta-keratin source material, less than about 0.04 endotoxin units per mg dry weight of cleaned beta-keratin source material, less than about 0.03 endotoxin units per mg dry weight of cleaned beta-keratin source material, less than about 0.02 endotoxin units per mg dry weight of cleaned beta-keratin source material or less than about 0.01 endotoxin units per mg dry weight of cleaned beta-keratin source material.

In some embodiments, the cleaned beta-keratin source material has less endotoxin units per mg dry weight of cleaned beta-keratin source material than is the detectable limits of the testing method. Testing can be performed using, for example, a kinetic chromogenic LAL assay as discussed in Example 2.

In some embodiments, the cleaned beta-keratin source material has from about 0.00004 to about 3.25 endotoxin units per mg of dry weight cleaned beta-keratin source material. In some embodiments, the cleaned beta-keratin source material has from about 0.00004 to about 3.0 endotoxin units per mg dry weight of cleaned beta-keratin source. In some embodiments, the cleaned beta-keratin source material has from about 0.00004 to about 2.8 endotoxin units per mg dry weight of cleaned beta-keratin source material, from about 0.00004 to about 2.6 endotoxin units per mg dry weight of cleaned beta-keratin source material, from about 0.00004 to about 2.4 endotoxin units per mg dry weight of cleaned beta-keratin source material, from about 0.00004 to about 2.2 endotoxin units per mg dry weight of cleaned beta-keratin source material, from about 0.001 to about 2.0 endotoxin units per mg dry weight of cleaned beta-keratin source material, from about 0.00004 to about 1.8 endotoxin units per mg dry weight of cleaned beta-keratin source material, from about 0.00004 to about 1.6 endotoxin units per mg dry weight of cleaned beta-keratin source material, from about 0.00004 to about 1.4 endotoxin units per mg dry weight of cleaned beta-keratin source material, from about 0.00004 to about 1.2 endotoxin units per mg dry weight of cleaned beta-keratin source material, and/or from about 0.00004 to about 1.0 endotoxin units per mg dry weight of cleaned beta-keratin source material.

In some embodiments, the cleaned beta-keratin source material has from about 0.00004 to about 0.90 endotoxin units per mg dry weight of cleaned beta-keratin source material, from about 0.00004 to about 0.80 endotoxin units per mg dry weight of cleaned beta-keratin source material, from about 0.00004 to about 0.70 endotoxin units per mg dry weight of cleaned beta-keratin source material, from about 0.00004 to about 0.60 endotoxin units per mg dry weight of cleaned beta-keratin source material, from about 0.00004 to about 0.50 endotoxin units per mg dry weight of cleaned beta-keratin source material, from about 0.00004 to about 0.40 endotoxin units per mg dry weight of cleaned beta-keratin source material, from about 0.00004 to about 0.30 endotoxin units per mg dry weight of cleaned beta-keratin source material, from about 0.00004 to about 0.20 endotoxin units per mg dry weight of cleaned beta-keratin source material, and/or from about 0.00004 to about 0.10 endotoxin units per mg dry weight of cleaned beta-keratin source material.

In some embodiments, the cleaned beta-keratin source material has from about 0.01 to about 0.10 endotoxin units per mg dry weight of cleaned beta-keratin source material, from about 0.01 to about 0.09 endotoxin units per mg dry weight of cleaned beta-keratin source material, from about 0.01 to about 0.08 endotoxin units per mg dry weight of cleaned beta-keratin source material, from about 0.01 to about 0.07 endotoxin units per mg dry weight of cleaned beta-keratin source material, from about 0.01 to about 0.06 endotoxin units per mg dry weight of cleaned beta-keratin source material, from about 0.01 to about 0.05 endotoxin units per mg dry weight of cleaned beta-keratin source material, from about 0.01 to about 0.04 endotoxin units per mg dry weight of cleaned beta-keratin source material, from about 0.01 to about 0.03 endotoxin units per mg dry weight of cleaned beta-keratin source material, and/or from about 0.01 to about 0.02 endotoxin units per mg dry weight of cleaned beta-keratin source material.

In some embodiments, the cleaned beta-keratin source material is subjected to a method of extracting keratin and/or a method of preparing a keratin extract. In some embodiments, the method comprises a reductive extraction. In some embodiments, the method comprises an oxidation extraction. In some embodiments, the method is a method disclosed here.

### III. Exemplary Embodiments of Protein Extracts

In one aspect, protein extracts are disclosed herein.

As discussed above, keratin is extracted from sources that contain contaminants such as endotoxins. Medical devices and biological products in the U.S. must meet FDA requirements for endotoxin levels as endotoxin contaminants in these products may cause unwanted and/or negative effects on the end user.

Accordingly, in some embodiments, disclosed herein is a protein extract comprising beta-keratin monomers, wherein the extract has less than about 10.0 endotoxin units per mg of extract. In some embodiments, the extract has less than 10.0 endotoxin units per mg of extract. In some embodiments, the extract has less than about 9.8 endotoxin units per mg of extract. In some embodiments, the extract has less than about 9.5 endotoxin units per mg of extract. In some embodiments, the extract has less than about 9.0 endotoxin units per mg of extract, less than about 8.5 endotoxin units per mg of extract, less than about 8.0 endotoxin units per mg of extract, less than about 7.5 endotoxin units per mg of extract, less than about 7.0 endotoxin units per mg of extract, less than about 6.5 endotoxin units per mg of extract, less than about 6 endotoxin units per mg of extract, less than about 5.5 endotoxin units per mg of extract, less than about 5.0 endotoxin units per mg of extract, less than about 4.5 endotoxin units per mg of extract, less than about 4.0 endotoxin units per mg of extract, less than about 3.5 endotoxin units per mg of extract, less than about 3.0 endotoxin units per mg of extract, less than about 2.5 endotoxin units per mg of extract, less than about 2.0 endotoxin units per mg of extract, less than about 1.5 endotoxin units per mg of extract, less than about 1.0 endotoxin units per mg of extract, and/or less than about 0.5 endotoxin units per mg of extract.

In some embodiments, the extract has less than about 0.80 endotoxin units per mg of extract, less than about 0.70 endotoxin units per mg of extract, less than about 0.60 endotoxin units per mg of extract, less than about 0.50 endotoxin units per mg of extract, less than about 0.4 endotoxin units per mg of extract, less than about 0.30 endotoxin units per mg of extract, less than about 0.20 endotoxin units per mg of extract, and/or less than about 0.10 endotoxin units per mg of extract.

In some embodiments, the extract has less than about 0.10 endotoxin units per mg of extract, less than about 0.5 endotoxin units per mg of extract, less than about 0.01 endotoxin units per mg of extract, less than about 0.005 endotoxin units per mg of extract, and/or less than about 0.001 endotoxin units per mg of extract. In some embodiments, the extract has less endotoxin units per mg than the detectable limits of the testing method. Testing can be performed using, for example, a kinetic chromogenic LAL assay as discussed in Examples 2 and 9.

In some embodiments, the extract has from about 0.001 to about 10.0 endotoxin units per mg of extract. In some embodiments, the extract has from about 0.001 to 10.0 endotoxin units per mg of extract. In some embodiments, the extract has from about 0.001 to about 9.5 endotoxin units per mg of extract. In some embodiments, the extract has from about 0.001 to about 9.0 endotoxin units per mg of extract, from about 0.001 to about 8.5 endotoxin units per mg of extract, from about 0.001 to about 8.0 endotoxin units per mg of extract, from about 0.001 to about 7.5 endotoxin units per mg of extract, from about 0.001 to about 7.0 endotoxin units per mg of extract, from about 0.001 to about 6.5 endotoxin units per mg of extract, from about 0.001 to about 6.0 endotoxin units per mg of extract, from about 0.001 to about 5.5 endotoxin units per mg of extract, from about 0.001 to about 5.0 endotoxin units per mg of extract, from about 0.001 to about 4.5 endotoxin units per mg of extract, from about 0.001 to about 4.0 endotoxin units per mg of extract, from about 0.001 to about 3.5 endotoxin units per mg of extract, from about 0.001 to about 3.0 endotoxin units per mg of extract, from about 0.001 to about 2.5 endotoxin units per mg of extract, from about 0.001 to about 2.0 endotoxin units per mg of extract, from about 0.001 to about 1.5 endotoxin units per mg of extract, and/or from about 0.001 to about 1.0 endotoxin unit per mg of extract.

In some embodiments, the extract has from about 0.001 to about 0.80 endotoxin units per mg of extract. In some embodiments, the extract has from about 0.001 to 0.70 endotoxin units per mg of extract. In some embodiments, the extract has from about 0.001 to about 0.60 endotoxin units per mg of extract. In some embodiments, the extract has from about 0.001 to about 0.50 endotoxin units per mg of extract, from about 0.001 to about 0.40 endotoxin units per mg of extract, from about 0.001 to about 0.30 endotoxin units per mg of extract, from about 0.001 to about 0.20 endotoxin units per mg of extract, from about 0.001 to about 0.10 endotoxin units per mg of extract, from about 0.001 to about 0.09 endotoxin units per mg of extract, from about 0.001 to about 0.08 endotoxin units per mg of extract, from about 0.001 to about 0.07 endotoxin units per mg of extract, from about 0.001 to about 0.06 endotoxin units per mg of extract, from about 0.001 to about 0.05 endotoxin units per mg of extract, from about 0.001 to about 0.04 endotoxin units per mg of extract, from about 0.001 to about 0.03 endotoxin units per mg of extract, from about 0.001 to about 0.02 endotoxin units per mg of extract, from about 0.001 to about 0.01 endotoxin units per mg of extract, from about 0.001 to about 2 endotoxin units per mg of extract, from about 0.001 to about 0.009 endotoxin units per mg of extract, and/or from about 0.001 to about 0.008 endotoxin unit per mg of extract.

In some embodiments, the extract has from about 0.20 to about 10.0 endotoxin units per mg of extract. In some embodiments, the extract has from about 0.20 to 10.0 endotoxin units per mg of extract. In some embodiments, the extract has from about 0.20 to about 9.5 endotoxin units per mg of extract. In some embodiments, the extract has from about 0.20 to about 9.0 endotoxin units per mg of extract, from about 0.20 to about 8.5 endotoxin units per mg of extract, from about 0.20 to about 8.0 endotoxin units per mg of extract, from about 0.20 to about 7.5 endotoxin units per mg of extract, from about 0.20 to about 7.0 endotoxin units per mg of extract, from about 0.20 to about 6.5 endotoxin units per mg of extract, from about 0.20 to about 6.0 endotoxin units per mg of extract, from about 0.20 to about 5.5 endotoxin units per mg of extract, from about 0.20 to about 5.0 endotoxin units per mg of extract, from about 0.20 to about 4.5 endotoxin units per mg of extract, from about 0.20 to about 4.0 endotoxin units per mg of extract, from about 0.20 to about 3.5 endotoxin units per mg of extract, from about 0.20 to about 3.0 endotoxin units per mg of extract, from about 0.20 to about 2.5 endotoxin units per mg of extract, from about 0.20 to about 2.0 endotoxin units per mg of extract, from about 0.20 to about 1.5 endotoxin units per mg of extract, and/or from about 0.20 to about 1.0 endotoxin unit per mg of extract.

In some embodiments, the extract has from about 0.20 to about 0.80 endotoxin units per mg of extract. In some embodiments, the extract has from about 0.20 to 0.70 endotoxin units per mg of extract. In some embodiments, the extract has from about 0.20 to about 0.60 endotoxin units per mg of extract. In some embodiments, the extract has from about 0.20 to about 0.50 endotoxin units per mg of extract, from about 0.20 to about 0.40 endotoxin units per mg of extract, from about 0.20 to about 0.30 endotoxin units per mg of extract.

Disulfide bonds contribute to the physical and functional properties of keratin networks. Free thiol groups in keratin monomers and/or oligomers can crosslink to form disulfide bonds and thus the amount of free thiols in a keratin extract is important in order to allow the formation of strong networks.

In some embodiments, disclosed herein is a protein extract comprising beta-keratin monomers, wherein the extract has at least about 0.20 mM free thiol groups when measured at about 0.1% weight per volume using Ellman's assay. In some embodiments, the extract has at least about 0.30 mM free thiol groups when measured at about 0.1% weight per volume using Ellman's assay, at least about 0.40 mM free thiol groups when measured at about 0.1% weight per volume using Ellman's assay, at least about 0.50 mM free thiol groups when measured at about 0.1% weight per volume using Ellman's assay, at least about 0.60 mM free thiol groups when measured at about 0.1% weight per volume using Ellman's assay, at least about 0.70 mM free thiol groups when measured at about 0.1% weight per volume using Ellman's assay, at least about 0.80 mM free thiol groups when measured at about 0.1% weight per volume using Ellman's assay, at least about 0.90 mM free thiol groups when measured at about 0.1% weight per volume using Ellman's assay, and/or at least about 1.00 mM free thiol groups when measured at about 0.1% weight per volume using Ellman's assay.

In some embodiments, the extract has from about 0.20 mM to about 1.50 mM free thiol groups when measured at about 0.1% weight per volume using Ellman's assay. In some embodiments, the extract has from about 0.20 mM to about 1.25 mM free thiol groups when measured at about 0.1% weight per volume using Ellman's assay, from about 0.20 mM to about 1.00 mM free thiol groups when measured at about 0.1% weight per volume using Ellman's assay, and/or from about 0.20 mM to about 0.50 mM free thiol groups when measured at about 0.1% weight per volume using Ellman's assay.

In some embodiments, the extract has about 0.5 mM thiol groups when measured at about 0.1% weight per volume using Ellman's assay. In some embodiments, the extract has about 0.6 mM free thiol groups when measured at about 0.1% weight per volume using Ellman's assay.

In some embodiments, disclosed herein is a protein extract comprising beta-keratin monomers, wherein the extract has above 0.16 mM free thiol groups when measured at about 1% weight per volume using Ellman's assay. In some embodiments, the extract has above 0.17 mM free thiol groups when measured at about 1% weight per volume using Ellman's assay, above 0.18 mM free thiol groups when measured at about 1% weight per volume using Ellman's assay, and/or above 0.19 mM free thiol groups when measured at about 1% weight per volume using Ellman's assay. In some embodiments, disclosed herein is a protein extract comprising beta-keratin monomers, wherein the extract has at least 0.20 mM free thiol groups when measured at about 1% weight per volume using Ellman's assay.

In some embodiments, the extract has at least 0.30 mM free thiol groups when measured at about 1% weight per volume using Ellman's assay, at least about 0.40 mM free thiol groups when measured at about 1% weight per volume using Ellman's assay, at least about 0.50 mM free thiol groups when measured at about 1% weight per volume using Ellman's assay, at least about 0.60 mM free thiol groups when measured at about 1% weight per volume using Ellman's assay, at least about 0.70 mM free thiol groups when measured at about 1% weight per volume using Ellman's assay, at least about 0.80 mM free thiol groups when measured at about 1% weight per volume using Ellman's assay, at least about 0.90 mM free thiol groups when measured at about 1% weight per volume using Ellman's assay, at least about 1.00 mM free thiol groups when measured at about 0.1% weight per volume using Ellman's assay.

In some embodiments, the extract has from 0.20 mM to about 2.00 mM free thiol groups when measured at about 1% weight per volume using Ellman's assay. In some embodiments, the extract has from 0.20 mM to about 1.50 mM free thiol groups when measured at about 1% weight per volume using Ellman's assay, from 0.20 mM to about 1.00 mM free thiol groups when measured at about 1% weight per volume using Ellman's assay, and/or from 0.20 mM to about 0.50 mM free thiol groups when measured at about 1% weight per volume using Ellman's assay.

In some embodiments, the extract has 0.20 mM free thiol groups when measured at about 1% weight per volume using Ellman's assay. In some embodiments, the extract has about 1.0 mM free thiol groups when measured at about 1% weight per volume using Ellman's assay.

Ellman's assay is described in more detail below in Example 6.

In some embodiments, disclosed herein is a protein extract comprising beta-keratin monomers, wherein the extract is in the form of a liquid. In some embodiments, disclosed herein is a protein extract comprising beta-keratin monomers, wherein the extract is in the form of a solid.

In some embodiments, disclosed herein is a protein extract comprising beta-keratin monomers, wherein the extract is dehydrated. Dehydration can be accomplished by known techniques including heating, air drying, drying at specific humidity, and lyophilization.

Keratin extracts are generally not soluble in neutral and near neutral pH solvents. Instead, extracts often require the use of strong acids or bases, which may cause skin irritation or a decrease in overall biocompatibility of a keratin containing biomaterial. Further, the use of strong solvents, such as acids or bases, to solubilize keratin extracts may adversely impact the structure of the keratin monomers and/or oligomers, which in turn may impact network formation and performance of keratin compositions. Solubilization in neutral or near neutral pH solvents is therefore preferable for maintaining protein integrity, facilitating keratin network formation, and maintaining biocompatibility of a keratin containing biomaterial.

Accordingly, in some embodiments, disclosed herein is a protein extract comprising beta-keratin monomers, wherein the extract when dehydrated is soluble or slightly soluble in a solvent having a pH from about 7 to about 8 at about 2.5% weight per volume. In some embodiments, the solvent having a pH from about 7 to about 8 is chosen from water and a buffer. In some embodiments, the buffer comprises a PBS buffer, an acetate buffer, a HEPES buffer, a Tris buffer, or a citrate buffer. In some embodiments, the PBS buffer has a pH of about 7. In some embodiments, the PBS buffer has a pH of about 8.

In some embodiments, the extract when dehydrated is soluble in water at about 2.5% weight per volume. In some embodiments, the extract when dehydrated is soluble in water at about 3% weight per volume. In some embodiments, the extract when dehydrated is soluble in water at about 3.5%, at about 4%, at about 4.5%, at about 5%, and/or at about 5.5% weight per volume. In some embodiments, the extract when dehydrated is soluble in water at about 5% weight per volume.

In some embodiments, the protein extract according to any of the embodiments herein is lyophilized. In some embodiments, the protein extract is a homogenized lyophilized extract.

In some embodiments, the extract comprises beta-keratin oligomers. In some embodiments, the extract comprises alpha-keratin monomers. In some embodiments, the extract comprises alpha-keratin oligomers.

In some embodiments, the extract comprises cysteine modified keratin. In some embodiments, the extract comprises oxidized keratin. In some embodiments, the extract does not comprise cysteine modified keratin. In some embodiments, the extract does not comprise oxidized keratin.

Residual moisture, or remaining residual water, of keratin extracts and compositions is measured in order to ensure sufficient dehydration of the material. Excess moisture reduces stability of the keratin extracts and may cause unwanted degradation.

Accordingly, in some embodiments, disclosed herein is an extract when dehydrated has a residual moisture content of less than about 25%. In some embodiments, the extract has a residual moisture content of less than about 20%. Residual moisture can be measured, for example, by placing a sample into a glass vial and stored in a desiccator until use. The weight is then recorded using an analytical balance. A blank sample vial is prepared and treated in the same manner as the sample. The sample and blank are heated to 100° C. and a needle is used to pierce the cap of each vial. The needle washes dry nitrogen across the sample and through a transfer line into a titrator such as a Mettler-Toledo, C30 Coulometric Titrator. The titration continues until all the moisture transfer is complete. The blank value is then subtracted from the test sample to ensure that the system suitability control is within 10% of the true value of the standard. The loss in sample mass, calculated by subtracting the final sample weight from the initial sample weight, is attributed to the residual moisture content of the sample.

In some embodiments, disclosed herein is a protein extract comprising beta-keratin monomers, wherein the extract has a combination of any or all of the embodiments recited above.

### IV. Exemplary Embodiments of Methods of Extracting Keratin, Methods of Preparing a Keratin Extract, and Keratin Extracts Prepared According to Those Methods

While various methods of extracting keratin monomers are known in the art, there are also known problems. For example, known processes result in keratin extracts that, after lyophilization, are difficult to solubilize in neutral and near neutral pH solvents. Further, the extracted keratin has low concentrations of free thiol groups, which are important for network formation. In some instances, the extract processes result in such low yield that they are impractical for commercial processes.

Accordingly, in one aspect, methods of extracting keratin monomers are disclosed herein. In some embodiments, the keratin monomers are beta-keratin monomers. In one aspect, methods of preparing a keratin extract are disclosed herein. In some embodiments, the keratin extract is a beta-keratin protein extract. In some embodiments, the methods of extracting beta-keratin monomers comprise mixing a beta-keratin source material with an extraction solution.

In some embodiments, the methods comprise purification of the protein extract. In some embodiments, the method comprises purifying the protein extract by filtration, buffer exchange, and/or chromatography. In some embodiments, the method comprises removal of unwanted materials, such as endotoxins, small molecules, lipids, surfactants, viral contaminants, unwanted proteins, unwanted tissue components, and processing reagents. In some embodiments, the unwanted material is a detergent, such as sodium dodecyl sulfate. In some embodiments, the unwanted material is a reducing agent, such as thioglycolic acid. In some embodiments, the purification removes more than one unwanted material.

In some embodiments, the methods comprise washing the beta-keratin source material prior to mixing the beta-keratin source material with the extraction solution. In some embodiments, the methods comprise defatting the beta-keratin source material prior to mixing the beta-keratin source material with the extraction solution.

In one aspect, keratin extracts are disclosed herein that are prepared according to the methods disclosed herein. In some embodiments, the keratin extract is a beta-keratin protein extract.

### A. Exemplary Embodiments of Methods of Extracting Keratin

In some embodiments, the method of extracting beta-keratin monomers comprises mixing a beta-keratin source material with an extraction solution comprising urea and thioglycolic acid. In some embodiments, the urea is at a concentration of at least about 1 M. In some embodiments, the urea is at a concentration of at least about 2 M, at least about 3 M, at least about 4 M, at least about 5 M, at least about 6 M, at least about 7 M, at least about 8 M, and/or at least about 9 M. In some embodiments, the urea concentration is at least about 5 M. In some embodiments, the urea concentration is at least about 5 M to about 9 M. In some embodiments, the urea concentration is at least about 6 M to about 8 M. In some embodiments, the urea concentration is at least about 7 M to about 8 M. In some embodiments, the urea concentration is at least about 8 M.

In some embodiments, the thioglycolic acid is at a concentration of about 0.5 M to about 1.5 M. In some embodiments, the thioglycolic acid is at a concentration of about 0.5 M, about 0.6 M, about 0.7 M, about 0.8 M, about 0.9 M, about 1.0 M, about 1.1 M, about 1.2 M, about 1.3 M, about 1.4 M, and/or about 1.5 M. In some embodiments, the thioglycolic acid is at a concentration of about 1.3 M.

In some embodiments, the pH of the extraction solution is from about 7 to about 10. In some embodiments, the pH of the extraction solution is about 7, about 8, about 9 or about 10. In some embodiments, the pH of the extraction solution is about 10. In some embodiments, buffers are used to adjust the pH. In some embodiments, the buffer comprises tris(hydroxymethyl)aminomethane (“Tris”), phosphate, glycine, HEPES, citrate, and carbonate.

In some embodiments, the ratio of beta-keratin source material to extraction solution is, or is equivalent to, from about 1 g to about 500 g dry weight beta-keratin source material per about 1000 mL extraction solution. In some embodiments, the ratio of beta-keratin source material to extraction solution is, or is equivalent to, from about 1 g to about 400 g dry weight beta-keratin source material per about 1000 mL extraction solution. In some embodiments, the ratio of beta-keratin source material to extraction solution is, or is equivalent to, from about 1 g to about 300 g dry weight beta-keratin source material per about 1000 mL extraction solution. In some embodiments, the ratio of beta-keratin source material to extraction solution is, or is equivalent to, from about 1 g to about 200 g dry weight beta-keratin source material per about 1000 mL extraction solution. In some embodiments, the ratio of beta-keratin source material to extraction solution is, or is equivalent to, from about 1 g to about 100 g dry weight beta-keratin source material per about 1000 mL extraction solution. In some embodiments, the ratio of beta-keratin source material to extraction solution is, or is equivalent to, from about 1 g to about 90 g dry weight beta-keratin source material per about 1000 mL extraction solution. In some embodiments, the ratio of beta-keratin source material to extraction solution is, or is equivalent to, from about 1 g to about 80 g, from about 1 g to about 70 g, from about 1 g to about 60 g, from about 1 g to about 50 g, from about 1 g to about 40 g, from about 1 to about 30 g, from about 1 g to about 20 mg, and/or from about 1 g to about 10 g dry weight beta-keratin source material per about 1000 mL extraction solution.

In some embodiments, the ratio of beta-keratin source material to extraction solution is, or is equivalent to, from about 1 g to about 30 g dry weight beta-keratin source material per about 1000 mL extraction solution. In some embodiments, the ratio is, or is equivalent to, from about 5 g to about 30 g dry weight beta-keratin source material per about 1000 mL extraction solution. In some embodiments, the ratio is, or is equivalent to, from about 10 g to about 30 g, from about 15 g to about 30 g, from about 20 g to about 30 g, and/or from about 25 g to about 30 g dry weight beta-keratin source material per about 1000 mL extraction solution.

In some embodiments, the ratio of beta-keratin source material to extraction solution is, or is equivalent to, from about 1 g to about 20 g dry weight beta-keratin source material per about 1000 mL extraction solution. In some embodiments, the ratio is, or is equivalent to, from about 5 g to about 20 g, from about 10 g to about 30 g, from about 15 g to about 30 g, from about 20 g to about 30 g, and/or from about 25 g to about 30 g dry weight beta-keratin source material per about 1000 mL extraction solution.

In some embodiments, the ratio of beta-keratin source material to extraction solution is, or is equivalent to, about 1 g dry weight beta-keratin source material per about 1000 mL extraction solution. In some embodiments, the ratio is, or is equivalent to, about 2 g per about 1000 mL, about 3 g per about 1000 mL, about 4 g per 1000 mL, about 5 g per about 1000 mL, about 6 g per about 1000 mL, about 7 g per about 1000 mL, about 8 g per about 1000 mL, about 9 g per about 1000 mL, about 10 g per about 1000 mL, about 11 g per about 1000 mL, about 12 g per about 1000 mL, about 13 g per about 1000 mL, about 14 g per about 1000 mL, about 15 g per about 1000 mL, about 16 g per about 1000 mL, about 17 g per about 1000 mL, about 18 g per about 1000 mL, about 19 g per about 1000 mL, about 20 g per about 1000 mL, about 21 g per about 1000 mL, about 22 g per about 1000 mL, about 23 per about 1000 mL, about 24 g per about 1000 mL, about 25 g per about 1000 mL, about 26 g per about 1000 mL, about 27 g per about 1000 mL, about 28 g per about 1000 mL, about 29 g per about 1000 mL, and/or about 30 g dry weight beta-keratin source material per about 1000 mL extraction solution. In some embodiments, the ratio of beta-keratin source material to extraction solution is, or is equivalent to, about 15 g dry weight beta-keratin source material per about 1000 mL extraction solution.

In some embodiments, the extraction solution further comprises a chelating agent. In some embodiments, the chelating agent is chosen from ethylenediaminetetraacetic acid (“EDTA”), Ethylene glycol-bis-(2-aminoethyl)-N,N,N′, N′-tetraacetic acid (EGTA), N-(2-Hydroxyethyl)ethylenediamine-N, N′, N′-triacetic acid trisodium salt (HEDTA), nitrilotriacetic acid (NTA), and Triethanolamine (TEA). In some embodiments, the extraction solution further comprises an additional reducing agent. In some embodiments, the additional reducing agent is chosen from tris(2-carboxyethyl) phosphine and dithiothreitol. In some embodiments, the extraction solution further comprises a denaturing agent. In some embodiment, the denaturing agent is guanidine hydrochloride.

In some embodiments, the beta-keratin source material is mixed with the extraction solution for at least about 30 minutes. In some embodiments, the beta-keratin source material is mixed with the extraction solution for about 30 minutes to about 5 hours. In some embodiments, the beta-keratin source material is mixed with the extraction solution for about 30 minutes, for about 1 hour, for about 1.5 hours, for about 2 hours, for about 2.5 hours, for about 3 hours, for about 3.5 hours, for about 4 hours, for about 4.5 hours or for about 5 hours. In some embodiments, the beta-keratin source material is mixed with the extraction solution for about 2 hours.

In some embodiments, the temperature of the mixture of the beta-keratin source material and the extraction solution is from about room temperature to about 80° C. In some embodiments, the temperature is from about 30° C. to about 75° C. In some embodiments, the temperature is from about 40° C. to about 70° C. In some embodiments, the temperature is from about 50° C. to about 70° C. In some embodiments, the temperature is about 60° C. to about 70° C. In some embodiments, the temperature is about 25° C., about 30° C., about 40° C., about 50° C., about 60° C., about 70° C. or about 80° C. In some embodiments, the temperature is about 50° C. or about 60° C.

In some embodiments, the method further comprises combining the beta-keratin source material with a washing solution prior to mixing the beta-keratin source material with the extraction solution.

In some embodiments, the washing solution comprises at least one detergent. In some embodiments, the at least one detergent comprises an ionic detergent and/or a non-ionic detergent. In some embodiments, the at least one detergent comprises SDS and/or a nonionic detergent. In some embodiments, the nonionic detergent comprises a polyethylene glycol-based detergent. In some embodiments, the nonionic detergent comprises a polyethylene glycol tert-octylphenyl ether. In some embodiments, the nonionic detergent comprises a compound of formula I:

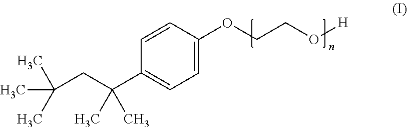

wherein n is chosen from 7-8, 9-10 or 40. In some embodiments, n is 7-8. In some embodiments, n is 9-10. In some embodiments, n is 40.

In some embodiments, the method further comprises a method of cleaning as disclosed in any or all of the above embodiments.

In some embodiments, the method further comprises defatting the beta-keratin source material prior to mixing the beta-keratin source material with the extraction solution. In some embodiments, the beta-keratin source material is defatted after washing the beta-keratin source material with a method of cleaning as disclosed herein.

In some embodiments, the beta-keratin source material is defatted after washing the beta-keratin source material with a washing solution. In some embodiments, the defatting step comprises soaking the beta-keratin source material in ethanol solution. In some embodiments, the ethanol solution comprises greater than about 10% ethanol. In some embodiments, the ethanol solution comprises greater than about 25% ethanol. In some embodiments, the ethanol solution comprises greater than about 50% ethanol. In some embodiments, the ethanol solution comprises greater than about 75% ethanol. In some embodiments, the ethanol solution comprises greater than about 90% ethanol. In some embodiments, the ethanol solution comprises about 95% ethanol. In some embodiments, the source material is soaked for from about 30 minutes to about 24 hours. In some embodiments, the source material is soaked for from about 1 minutes to about 12 hours. In some embodiments, the source material is soaked for from about 2 to about 4 hours.

In some embodiments, disclosed herein is a method further comprising purification of the product of any of the above methods, such as filtration, buffer exchange, and/or chromatography. In some embodiments, chromatography comprises separation of compounds, such as endotoxin and/or proteins, using one or more stationary phases. In some embodiments, buffer exchange comprises using one or more buffer exchange steps.

In some embodiments, the method further comprises filtering the product of any of the above methods to produce a filtrate. In some embodiments, the filtering comprises vacuum filtration, gravity filtration and/or by centrifugation. In some embodiments, the method further comprises dialyzing the filtrate to produce a dialyzed material. In some embodiments, the filtrate is dialyzed with water. In some embodiments, the dialyzing comprises dialyzing with a dialysis tube and/or by filtration. In some embodiments, the filtration comprises tangential flow filtration, membrane filtration, depth filtration, and/or ultrafiltration. In some embodiments, the dialyzing comprises dialyzing with a dialysis tube. In some embodiments, the dialysis tube has a molecular weight cut-off (MWCO) of from about 1 kDa to 500 Da. In some embodiments, the dialysis tube has a MWCO of about 3.5 kDa. In some embodiments, the filtrate is dialyzed for 3-5 days. In some embodiments, the method comprises diluting the filtrate prior to dialysis.

In some embodiments, the method further comprises performing a buffer exchange on the product of any of the above methods. In some embodiments, buffer exchange is accomplished by tangential flow filtration. In some embodiments, buffer exchange is accomplished by dialysis. In some embodiments, buffer exchange is accomplished by Size Exclusion Chromatography. In some embodiments, buffer exchange is accomplished by Depth Filtration.

In some embodiments, the method further comprises performing chromatography on the product of any of the above methods. In some embodiments, chromatography is accomplished by Ion Exchange Chromatography. In some embodiments, chromatography is accomplished by Hydrophobic Interaction Chromatography. In some embodiments, chromatography is accomplished by Mixed Mode Chromatography. In some embodiments, chromatography is accomplished by Hydroxyapatite Chromatography.

In some embodiments, the method comprises performing buffer exchange before performing chromatography. In some embodiments, the method comprises performing chromatography before performing buffer exchange. In some embodiments, the method comprises performing a first buffer exchange, followed by chromatography, followed by a second buffer exchange.

In some embodiments, the method further comprises lyophilizing the product of any of the methods above to produce a lyophilized material. In some embodiments, the method further comprises homogenizing the lyophilized material.

In some embodiments, the beta-keratin source material comprises epidermal appendages of reptiles and/or birds. In some embodiments, the beta-keratin source material comprises epidermal appendages of reptiles. In some embodiments, the beta-keratin source material comprises epidermal appendages of alligators. In some embodiments, the beta-keratin source material comprises alligator scales. In some embodiments, the beta-keratin source material comprises epidermal appendages of birds. In some embodiments, the beta-keratin source material comprises avian source material. In some embodiments, the beta-keratin source material comprises feathers. In some embodiments, the beta-keratin source material comprises goose and/or chicken feathers. In some embodiments, the beta-keratin source material comprises white chicken feathers. In some embodiments, the beta-keratin source material comprises brown chicken feathers. In some embodiments, the beta-keratin source material comprises black chicken feathers.

In some embodiments, the extracted beta-keratin protein produced by the methods herein has less than about 10.0 endotoxin units per mg of extracted beta-keratin protein. In some embodiments, the extract has less than 10.0 endotoxin units per mg of extract. In some embodiments, the extract has less than about 9.8 endotoxin units per mg of extract. In some embodiments, the extract has less than about 9.5 endotoxin units per mg of extract. In some embodiments, the extract has less than about 9.0 endotoxin units per mg of extract, less than about 8.5 endotoxin units per mg of extract, less than about 8.0 endotoxin units per mg of extract, less than about 7.5 endotoxin units per mg of extract, less than about 7.0 endotoxin units per mg of extract, less than about 6.5 endotoxin units per mg of extract, less than about 6.0 endotoxin units per mg of extract, less than about 5.5 endotoxin units per mg of extract, less than about 5.0 endotoxin units per mg of extract, less than about 4.5 endotoxin units per mg of extract, less than about 4.0 endotoxin units per mg of extract, less than about 3.5 endotoxin units per mg of extract, less than about 3.0 endotoxin units per mg of extract, less than about 2.5 endotoxin units per mg of extract, less than about 2.0 endotoxin units per mg of extract, less than about 1.5 endotoxin units per mg of extract, less than about 1 endotoxin units per mg of extract, and/or less than about 0.5 endotoxin units per mg of extract.

In some embodiments, the extracted beta-keratin protein produced by the methods herein has less than about 0.80 endotoxin units per mg of extract, less than about 0.70 endotoxin units per mg of extract, less than about 0.60 endotoxin units per mg of extract, less than about 0.50 endotoxin units per mg of extract, less than about 0.40 endotoxin units per mg of extract, less than about 0.30 endotoxin units per mg of extract, less than about 0.20 endotoxin units per mg of extract, and/or less than about 0.10 endotoxin units per mg of extract.

In some embodiments, the extracted beta-keratin protein has less than about 0.10 endotoxin units per mg of extract, less than about 0.50 endotoxin units per mg of extract, less than about 0.01 endotoxin units per mg of extract, less than about 0.005 endotoxin units per mg of extract, and/or less than about 0.001 endotoxin units per mg of extract. In some embodiments, the extract has less endotoxin units per mg than the detectable limits of the testing method. Testing can be performed using, for example, a kinetic chromogenic LAL assay as discussed in Examples 2 and 9.

In some embodiments, the extracted beta-keratin protein has from about 0.001 to about 10.0 endotoxin units per mg of extract. In some embodiments, the extract has from about 0.001 to 10.0 endotoxin units per mg of extract. In some embodiments, the extract has from about 0.001 to about 9.5 endotoxin units per mg of extract. In some embodiments, the extract has from about 0.001 to about 9.0 endotoxin units per mg of extract, from about 0.001 to about 8.5 endotoxin units per mg of extract, from about 0.001 to about 8.0 endotoxin units per mg of extract, from about 0.001 to about 7.5 endotoxin units per mg of extract, from about 0.001 to about 7.0 endotoxin units per mg of extract, from about 0.001 to about 6.5 endotoxin units per mg of extract, from about 0.001 to about 6.0 endotoxin units per mg of extract, from about 0.001 to about 5.5 endotoxin units per mg of extract, from about 0.001 to about 5.0 endotoxin units per mg of extract, from about 0.001 to about 4.5 endotoxin units per mg of extract, from about 0.001 to about 4.0 endotoxin units per mg of extract, from about 0.001 to about 3.5 endotoxin units per mg of extract, from about 0.001 to about 3.0 endotoxin units per mg of extract, from about 0.001 to about 2.5 endotoxin units per mg of extract, from about 0.001 to about 2.0 endotoxin units per mg of extract, from about 0.001 to about 1.5 endotoxin units per mg of extract, and/or from about 0.001 to about 1.0 endotoxin unit per mg of extract.

In some embodiments, the extracted beta-keratin protein has from about 0.001 to about 0.80 endotoxin units per mg of extract. In some embodiments, the extract has from about 0.001 to 0.70 endotoxin units per mg of extract. In some embodiments, the extract has from about 0.001 to about 0.60 endotoxin units per mg of extract. In some embodiments, the extract has from about 0.001 to about 0.50 endotoxin units per mg of extract, from about 0.001 to about 0.40 endotoxin units per mg of extract, from about 0.001 to about 0.30 endotoxin units per mg of extract, from about 0.001 to about 0.20 endotoxin units per mg of extract, from about 0.001 to about 0.10 endotoxin units per mg of extract, from about 0.001 to about 0.09 endotoxin units per mg of extract, from about 0.001 to about 0.08 endotoxin units per mg of extract, from about 0.001 to about 0.07 endotoxin units per mg of extract, from about 0.001 to about 0.06 endotoxin units per mg of extract, from about 0.001 to about 0.05 endotoxin units per mg of extract, from about 0.001 to about 0.04 endotoxin units per mg of extract, from about 0.001 to about 0.03 endotoxin units per mg of extract, from about 0.001 to about 0.02 endotoxin units per mg of extract, from about 0.001 to about 0.01 endotoxin units per mg of extract, from about 0.001 to about 2 endotoxin units per mg of extract, from about 0.001 to about 0.009 endotoxin units per mg of extract, and/or from about 0.001 to about 0.008 endotoxin unit per mg of extract.

In some embodiments, the extracted beta-keratin protein has from about 0.20 to about 10.0 endotoxin units per mg of extract. In some embodiments, the extract has from about 0.20 to 10.0 endotoxin units per mg of extract. In some embodiments, the extract has from about 0.20 to about 9.5 endotoxin units per mg of extract. In some embodiments, the extract has from about 0.20 to about 9.0 endotoxin units per mg of extract, from about 0.20 to about 8.5 endotoxin units per mg of extract, from about 0.20 to about 8.0 endotoxin units per mg of extract, from about 0.20 to about 7.5 endotoxin units per mg of extract, from about 0.20 to about 7.0 endotoxin units per mg of extract, from about 0.20 to about 6.5 endotoxin units per mg of extract, from about 0.20 to about 6.0 endotoxin units per mg of extract, from about 0.20 to about 5.5 endotoxin units per mg of extract, from about 0.20 to about 5.0 endotoxin units per mg of extract, from about 0.20 to about 4.5 endotoxin units per mg of extract, from about 0.20 to about 4.0 endotoxin units per mg of extract, from about 0.20 to about 3.5 endotoxin units per mg of extract, from about 0.20 to about 3.0 endotoxin units per mg of extract, from about 0.20 to about 2.5 endotoxin units per mg of extract, from about 0.20 to about 2.0 endotoxin units per mg of extract, from about 0.20 to about 1.5 endotoxin units per mg of extract, and/or from about 0.20 to about 1.0 endotoxin unit per mg of extract.

In some embodiments, the extracted beta-keratin protein has from about 0.20 to about 0.80 endotoxin units per mg of extract. In some embodiments, the extract has from about 0.20 to 0.70 endotoxin units per mg of extract. In some embodiments, the extract has from about 0.20 to about 0.60 endotoxin units per mg of extract. In some embodiments, the extract has from about 0.20 to about 0.50 endotoxin units per mg of extract, from about 0.20 to about 0.40 endotoxin units per mg of extract, from about 0.20 to about 0.30 endotoxin units per mg of extract.

In some embodiments, the extracted beta-keratin protein has at least about 0.20 mM free thiol groups when measured at about 0.1% weight per volume using Ellman's assay. In some embodiments, the extract has at least about 0.30 mM free thiol groups when measured at about 0.1% weight per volume using Ellman's assay, at least about 0.40 mM free thiol groups when measured at about 0.1% weight per volume using Ellman's assay, at least about 0.50 mM free thiol groups when measured at about 0.1% weight per volume using Ellman's assay, at least about 0.60 mM free thiol groups when measured at about 0.1% weight per volume using Ellman's assay, at least about 0.70 mM free thiol groups when measured at about 0.1% weight per volume using Ellman's assay, at least about 0.80 mM free thiol groups when measured at about 0.1% weight per volume using Ellman's assay, at least about 0.90 mM free thiol groups when measured at about 0.1% weight per volume using Ellman's assay, and/or at least about 1.00 mM free thiol groups when measured at about 0.1% weight per volume using Ellman's assay.

In some embodiments, the extracted beta-keratin protein has from about 0.20 mM to about 1.50 mM free thiol groups when measured at about 0.1% weight per volume using Ellman's assay. In some embodiments, the extract has from about 0.20 mM to about 1.25 mM free thiol groups when measured at about 0.1% weight per volume using Ellman's assay, from about 0.20 mM to about 1.00 mM free thiol groups when measured at about 0.1% weight per volume using Ellman's assay, and/or from about 0.20 mM to about 0.50 mM free thiol groups when measured at about 0.1% weight per volume using Ellman's assay.

In some embodiments, the extracted beta-keratin protein has at least about 0.50 mM thiol groups when measured at about 0.1% weight per volume using Ellman's assay. In some embodiments, the extract has about 0.6 mM free thiol groups when measured at about 0.1% weight per volume using Ellman's assay.

In some embodiments, disclosed herein is an extracted beta-keratin protein comprising beta-keratin monomers, wherein the extract has above 0.16 mM free thiol groups when measured at about 1% weight per volume using Ellman's assay. In some embodiments, the extract has above 0.17 mM free thiol groups when measured at about 1% weight per volume using Ellman's assay, above 0.18 mM free thiol groups when measured at about 1% weight per volume using Ellman's assay, and/or above 0.19 mM free thiol groups when measured at about 1% weight per volume using Ellman's assay. In some embodiments, the extracted beta-keratin protein has at least 0.20 mM free thiol groups when measured at about 1% weight per volume using Ellman's assay.

In some embodiments, the extract has at least 0.30 mM free thiol groups when measured at about 1% weight per volume using Ellman's assay, at least about 0.40 mM free thiol groups when measured at about 1% weight per volume using Ellman's assay, at least about 0.50 mM free thiol groups when measured at about 1% weight per volume using Ellman's assay, at least about 0.60 mM free thiol groups when measured at about 1% weight per volume using Ellman's assay, at least about 0.70 mM free thiol groups when measured at about 1% weight per volume using Ellman's assay, at least about 0.80 mM free thiol groups when measured at about 1% weight per volume using Ellman's assay, at least about 0.90 mM free thiol groups when measured at about 1% weight per volume using Ellman's assay, at least about 1.00 mM free thiol groups when measured at about 0.1% weight per volume using Ellman's assay.

In some embodiments, the extracted beta-keratin protein has from 0.20 mM to about 2.00 mM free thiol groups when measured at about 1% weight per volume using Ellman's assay. In some embodiments, the extract has from 0.20 mM to about 1.50 mM free thiol groups when measured at about 1% weight per volume using Ellman's assay, from 0.20 mM to about 1.00 mM free thiol groups when measured at about 1% weight per volume using Ellman's assay, and/or from 0.20 mM to about 0.50 mM free thiol groups when measured at about 1% weight per volume using Ellman's assay.

In some embodiments, the extracted beta-keratin protein has 0.20 mM free thiol groups when measured at about 1% weight per volume using Ellman's assay. In some embodiments, the extract has about 1.0 mM free thiol groups when measured at about 1% weight per volume using Ellman's assay.

Ellman's assay is described in more detail below in Example 6.

In some embodiments, the extracted beta-keratin protein when dehydrated is soluble or slightly soluble in a solvent having a pH from about 7 to about 8 at about 2.5% weight per volume. In some embodiments, the solvent having a pH from about 7 to about 8 is chosen from water and a buffer. In some embodiments, the buffer comprises a PBS buffer. In some embodiments, the PBS buffer has a pH of about 7. In some embodiments, the PBS buffer has a pH of about 8.

In some embodiments, the extract when dehydrated is soluble in water at about 2.5% weight per volume. In some embodiments, the extract when dehydrated is soluble in water at about 3% weight per volume. In some embodiments, the extract when dehydrated is soluble in water at about 3.5%, at about 4%, at about 4.5%, at about 5%, and/or at about 5.5% weight per volume. In some embodiments, the extract when dehydrated is soluble in water at about 5% weight per volume.

In some embodiments, the extracted beta-keratin protein is produced from about 10% to about 70%. In some embodiments, the extracted beta-keratin protein is produced from about 10% to about 60%. In some embodiments, the extracted beta-keratin protein is produced in about 20% to about 50% yield. In some embodiments, the extracted beta-keratin protein is produced in about 25% to about 45% yield. In some embodiments, the extracted beta-keratin protein is produced in about 30% to about 40% yield. In some embodiments, the extracted beta-keratin protein is produced in about 35% yield. In some embodiments, the yield is determined by comparing the mass of the lyophilized protein extract to the initial mass of dried feathers extracted.

In some embodiments, the method includes modifying the cysteine residues of the beta-keratin source material and/or the extracted beta-keratin material. In some embodiments, the method includes oxidizing the beta-keratin source material and/or the extracted beta-keratin material. In some embodiments, the method does not include modifying the cysteine residues of the beta-keratin source material and/or the extracted beta-keratin material. In some embodiments, the method does not include oxidizing the beta-keratin source material and/or the extracted beta-keratin material.

### B. Exemplary Embodiments of Methods of Preparing a Keratin Extract

In some embodiments, the method of preparing a beta-keratin protein extract comprises filtering the product of any one of previous methods to produce a filtrate. In some embodiments, the filtering comprises vacuum, gravity filtration and/or centrifugation.

In some embodiments, the method further comprises dialyzing the filtrate to produce a dialyzed material. In some embodiments, the filtrate is dialyzed with water. In some embodiments, the dialyzing comprises dialyzing with a dialysis tube and/or filtration. In some embodiments, the filtration comprises tangential flow filtration, membrane filtration, depth filtration, and/or ultrafiltration. In some embodiments, the dialyzing comprises dialyzing with a dialysis tube. In some embodiments, the dialysis tube has a molecular weight cut-off (MWCO) of from about 1 kDa to 500 Da. In some embodiments, the dialysis tube has a MWCO of about 3.5 kDa. In some embodiments, the filtrate is dialyzed for 3-5 days. In some embodiments, the method comprises diluting the filtrate prior to dialysis.

In some embodiments, the method of preparing a beta-keratin protein extract further comprises lyophilizing the filtrate and/or the dialyzed material to produce a lyophilized material. In some embodiments, the method further comprises homogenizing the lyophilized material.

In some embodiments, the methods herein include modifying the cysteine residues of the beta-keratin source and/or the extracted beta-keratin material. In some embodiments, the methods herein include oxidizing the beta-keratin source material and/or the extracted beta-keratin material. In some embodiments, the methods herein do not include modifying the cysteine residues of the beta-keratin source and/or the extracted beta-keratin material. In some embodiments, the methods herein do not include oxidizing the beta-keratin source material and/or the extracted beta-keratin material.

### C. Exemplary Embodiments of Keratin Extracts from Methods of Extracting Keratin and Methods of Preparing a Keratin Extract

In some embodiments, a beta-keratin protein extract is prepared according to any of the embodiments above.

In some embodiments, the beta-keratin protein extract has less than about 10.0 endotoxin units per mg of extract. In some embodiments, the extract has less than 10.0 endotoxin units per mg of extract. In some embodiments, the extract has less than about 9.8 endotoxin units per mg of extract. In some embodiments, the extract has less than about 9.5 endotoxin units per mg of extract. In some embodiments, the extract has less than about 9.0 endotoxin units per mg of extract, less than about 8.5 endotoxin units per mg of extract, less than about 8.0 endotoxin units per mg of extract, less than about 7.5 endotoxin units per mg of extract, less than about 7.0 endotoxin units per mg of extract, less than about 6.5 endotoxin units per mg of extract, less than about 6.0 endotoxin units per mg of extract, less than about 5.5 endotoxin units per mg of extract, less than about 5.0 endotoxin units per mg of extract, less than about 4.5 endotoxin units per mg of extract, less than about 4.0 endotoxin units per mg of extract, less than about 3.5 endotoxin units per mg of extract, less than about 3.0 endotoxin units per mg of extract, less than about 2.5 endotoxin units per mg of extract, less than about 2.0 endotoxin units per mg of extract, less than about 1.5 endotoxin units per mg of extract, less than about 1.0 endotoxin units per mg of extract, and/or less than about 0.5 endotoxin units per mg of extract.

In some embodiments, the beta-keratin protein extract has less than about 0.80 endotoxin units per mg of extract, less than about 0.70 endotoxin units per mg of extract, less than about 0.60 endotoxin units per mg of extract, less than about 0.50 endotoxin units per mg of extract, less than about 0.40 endotoxin units per mg of extract, less than about 0.30 endotoxin units per mg of extract, less than about 0.20 endotoxin units per mg of extract, and/or less than about 0.10 endotoxin units per mg of extract.

In some embodiments, the beta-keratin protein extract has less than about 0.10 endotoxin units per mg of extract, less than about 0.50 endotoxin units per mg of extract, less than about 0.01 endotoxin units per mg of extract, less than about 0.005 endotoxin units per mg of extract, and/or less than about 0.001 endotoxin units per mg of extract. In some embodiments, the extract has less endotoxin units per mg than the detectable limits of the testing method. Testing can be performed using, for example, a kinetic chromogenic LAL assay as discussed in Examples 2 and 9.

In some embodiments, the beta-keratin protein extract has from about 0.001 to about 10.0 endotoxin units per mg of extract. In some embodiments, the beta-keratin protein extract has from about 0.001 to 10.0 endotoxin units per mg of extract. In some embodiments, the extract has from about 0.001 to about 9.5 endotoxin units per mg of extract. In some embodiments, the extract has from about 0.001 to about 9.0 endotoxin units per mg of extract, from about 0.001 to about 8.5 endotoxin units per mg of extract, from about 0.001 to about 8.0 endotoxin units per mg of extract, from about 0.001 to about 7.5 endotoxin units per mg of extract, from about 0.001 to about 7.0 endotoxin units per mg of extract, from about 0.001 to about 6.5 endotoxin units per mg of extract, from about 0.001 to about 6.0 endotoxin units per mg of extract, from about 0.001 to about 5.5 endotoxin units per mg of extract, from about 0.001 to about 5.0 endotoxin units per mg of extract, from about 0.001 to about 4.5 endotoxin units per mg of extract, from about 0.001 to about 4.0 endotoxin units per mg of extract, from about 0.001 to about 3.5 endotoxin units per mg of extract, from about 0.001 to about 3.0 endotoxin units per mg of extract, from about 0.001 to about 2.5 endotoxin units per mg of extract, from about 0.001 to about 2.0 endotoxin units per mg of extract, from about 0.001 to about 1.5 endotoxin units per mg of extract, and/or from about 0.001 to about 1.0 endotoxin unit per mg of extract.

In some embodiments, the beta-keratin protein extract has from about 0.001 to about 0.80 endotoxin units per mg of extract. In some embodiments, the extract has from about 0.001 to 0.70 endotoxin units per mg of extract. In some embodiments, the extract has from about 0.001 to about 0.60 endotoxin units per mg of extract. In some embodiments, the extract has from about 0.001 to about 0.50 endotoxin units per mg of extract, from about 0.001 to about 0.40 endotoxin units per mg of extract, from about 0.001 to about 0.30 endotoxin units per mg of extract, from about 0.001 to about 0.20 endotoxin units per mg of extract, from about 0.001 to about 0.10 endotoxin units per mg of extract, from about 0.001 to about 0.09 endotoxin units per mg of extract, from about 0.001 to about 0.08 endotoxin units per mg of extract, from about 0.001 to about 0.07 endotoxin units per mg of extract, from about 0.001 to about 0.06 endotoxin units per mg of extract, from about 0.001 to about 0.05 endotoxin units per mg of extract, from about 0.001 to about 0.04 endotoxin units per mg of extract, from about 0.001 to about 0.03 endotoxin units per mg of extract, from about 0.001 to about 0.02 endotoxin units per mg of extract, from about 0.001 to about 0.01 endotoxin units per mg of extract, from about 0.001 to about 2 endotoxin units per mg of extract, from about 0.001 to about 0.009 endotoxin units per mg of extract, and/or from about 0.001 to about 0.008 endotoxin unit per mg of extract.

In some embodiments, the beta-keratin protein extract has from about 0.20 to about 10.0 endotoxin units per mg of extract. In some embodiments, the beta-keratin protein extract has from about 0.20 to 10.0 endotoxin units per mg of extract. In some embodiments, the extract has from about 0.20 to about 9.5 endotoxin units per mg of extract. In some embodiments, the extract has from about 0.20 to about 9.0 endotoxin units per mg of extract, from about 0.20 to about 8.5 endotoxin units per mg of extract, from about 0.20 to about 8.0 endotoxin units per mg of extract, from about 0.20 to about 7.5 endotoxin units per mg of extract, from about 0.20 to about 7.0 endotoxin units per mg of extract, from about 0.20 to about 6.5 endotoxin units per mg of extract, from about 0.20 to about 6.0 endotoxin units per mg of extract, from about 0.20 to about 5.5 endotoxin units per mg of extract, from about 0.20 to about 5.0 endotoxin units per mg of extract, from about 0.20 to about 4.5 endotoxin units per mg of extract, from about 0.20 to about 4.0 endotoxin units per mg of extract, from about 0.20 to about 3.5 endotoxin units per mg of extract, from about 0.20 to about 3.0 endotoxin units per mg of extract, from about 0.20 to about 2.5 endotoxin units per mg of extract, from about 0.20 to about 2.0 endotoxin units per mg of extract, from about 0.20 to about 1.5 endotoxin units per mg of extract, and/or from about 0.20 to about 1.0 endotoxin unit per mg of extract.

In some embodiments, the beta-keratin protein extract has at least about 0.20 mM free thiol groups when measured at about 0.1% weight per volume using Ellman's assay. In some embodiments, the extract has at least about 0.30 mM free thiol groups when measured at about 0.1% weight per volume using Ellman's assay, at least about 0.40 mM free thiol groups when measured at about 0.1% weight per volume using Ellman's assay, at least about 0.50 mM free thiol groups when measured at about 0.1% weight per volume using Ellman's assay, at least about 0.60 mM free thiol groups when measured at about 0.1% weight per volume using Ellman's assay, at least about 0.70 mM free thiol groups when measured at about 0.1% weight per volume using Ellman's assay, at least about 0.80 mM free thiol groups when measured at about 0.1% weight per volume using Ellman's assay, at least about 0.90 mM free thiol groups when measured at about 0.1% weight per volume using Ellman's assay, and/or at least about 1.00 mM free thiol groups when measured at about 0.1% weight per volume using Ellman's assay.

In some embodiments, the beta-keratin protein extract has from about 0.20 mM to about 1.50 mM free thiol groups when measured at about 0.1% weight per volume using Ellman's assay. In some embodiments, the extract has from about 0.20 mM to about 1.25 mM free thiol groups when measured at about 0.1% weight per volume using Ellman's assay, from about 0.20 mM to about 1.00 mM free thiol groups when measured at about 0.1% weight per volume using Ellman's assay, and/or from about 0.20 mM to about 0.50 mM free thiol groups when measured at about 0.1% weight per volume using Ellman's assay.

In some embodiments, the beta-keratin protein extract has about 0.5 mM free thiol groups when measured at about 0.1% weight per volume using Ellman's assay. In some embodiments, the extract has about 0.6 mM free thiol groups when measured at about 0.1% weight per volume using Ellman's assay.

In some embodiments, disclosed herein is a beta-keratin protein extract comprising beta-keratin monomers, wherein the extract has above 0.16 mM free thiol groups when measured at about 1% weight per volume using Ellman's assay. In some embodiments, the extract has above 0.17 mM free thiol groups when measured at about 1% weight per volume using Ellman's assay, above 0.18 mM free thiol groups when measured at about 1% weight per volume using Ellman's assay, and/or above 0.19 mM free thiol groups when measured at about 1% weight per volume using Ellman's assay. In some embodiments, the beta-keratin protein extract has at least 0.20 mM free thiol groups when measured at about 1% weight per volume using Ellman's assay.

In some embodiments, the extract has at least 0.30 mM free thiol groups when measured at about 1% weight per volume using Ellman's assay, at least about 0.40 mM free thiol groups when measured at about 1% weight per volume using Ellman's assay, at least about 0.50 mM free thiol groups when measured at about 1% weight per volume using Ellman's assay, at least about 0.60 mM free thiol groups when measured at about 1% weight per volume using Ellman's assay, at least about 0.70 mM free thiol groups when measured at about 1% weight per volume using Ellman's assay, at least about 0.80 mM free thiol groups when measured at about 1% weight per volume using Ellman's assay, at least about 0.90 mM free thiol groups when measured at about 1% weight per volume using Ellman's assay, at least about 1.00 mM free thiol groups when measured at about 0.1% weight per volume using Ellman's assay.

In some embodiments, the beta-keratin protein extract has from 0.20 mM to about 2.00 mM free thiol groups when measured at about 1% weight per volume using Ellman's assay. In some embodiments, the extract has from 0.20 mM to about 1.50 mM free thiol groups when measured at about 1% weight per volume using Ellman's assay, from 0.20 mM to about 1.00 mM free thiol groups when measured at about 1% weight per volume using Ellman's assay, and/or from 0.20 mM to about 0.50 mM free thiol groups when measured at about 1% weight per volume using Ellman's assay.

In some embodiments, the beta-keratin protein extract has 0.20 mM free thiol groups when measured at about 1% weight per volume using Ellman's assay. In some embodiments, the extract has about 1.0 mM free thiol groups when measured at about 1% weight per volume using Ellman's assay.

Ellman's assay is described in more detail below in Example 6.

In some embodiments, the beta-keratin protein extract when dehydrated is soluble or slightly soluble in a solvent having a pH from about 7 to about 8 at about 2.5% weight per volume when dehydrated. In some embodiments, the solvent having a pH from about 7 to about 8 is chosen from water and a buffer. In some embodiments, the buffer comprises a PBS buffer, an acetate buffer, a citrate buffer, a tris buffer, or a HEPES buffer. In some embodiments, the PBS buffer has a pH of about 7. In some embodiments, the PBS buffer has a pH of about 8.

In some embodiments, the beta-keratin protein extract when dehydrated is soluble in water at about 2.5% weight per volume. In some embodiments, the extract when dehydrated is soluble in water at about 3% weight per volume. In some embodiments, the extract when dehydrated is soluble in water at about 3.5%, at about 4%, at about 4.5%, at about 5%, and/or at about 5.5% weight per volume. In some embodiments, the extract when dehydrated is soluble in water at about 5% weight per volume.

In some embodiments, disclosed herein is a beta-keratin protein extract prepared according to any of the methods of extraction or methods of preparing a protein extract disclosed herein, where in the extract has a combination of any or all of the embodiments recited above.

### V. Exemplary Embodiments of Keratin Compositions

In one aspect, keratin compositions are disclosed herein. In some embodiments, the keratin composition comprises a beta-keratin extract. In some embodiments, the keratin composition comprises a beta-keratin network.

In order to meet FDA requirements for pyrogenicity, medical device products in the U.S. must contain ≤20 endotoxin units (“EU”) per device. Biological products must meet the FDA requirement of ≤5 EU per kg of body weight. Thus, if the average body weight of a human is 80 kg, the endotoxin limit for the biological product will be less than about 400 EU per product. To date there are no beta-keratin containing biomaterials or other products that are approved or cleared for use by the FDA.

In some embodiments, the keratin composition comprises a beta-keratin extract, wherein the keratin composition has less than about 400 endotoxin unit per composition. In some embodiments, the keratin composition comprises a beta-keratin network, wherein the keratin composition has less than about 400 endotoxin unit per composition. In some embodiments, the keratin composition has less than about 350 endotoxin units per composition, less than about 300 endotoxin units per composition, less than about 250 endotoxin units per composition, less than about 200 endotoxin units per composition, less than about 200 endotoxin units per composition, less than about 150 endotoxin units per composition, less than about 100 endotoxin units per composition, and/or less than about 50 endotoxin units per composition.

In some embodiments, the keratin composition has from about 1 to about 400 endotoxin units per composition. In some embodiments, the keratin composition has from about 1 to about 350 endotoxin units per composition, from about 1 to about 300 endotoxin units per composition, from about 1 to about 250 endotoxin units per composition, from about 1 to about 200 endotoxin units per composition, from about 1 to about 150 endotoxin units per composition, from about 1 to about 100 endotoxin units per composition, and/or from about 1 to about 50 endotoxin units per composition.

In some embodiments, the keratin composition has less than about 20.0 endotoxin units per composition. In some embodiments, the keratin composition has less than about 19.0 endotoxin units per composition, less than about 18.0 endotoxin units per composition, less than about 17.0 endotoxin units per composition, less than about 16.0 endotoxin units per composition, less than about 15.0 endotoxin units per composition, less than about 14.0 endotoxin units per composition, less than about 13.0 endotoxin units per composition, less than about 12.0 endotoxin units per composition, less than about 11.0 endotoxin units per composition, less than about 10.0 endotoxin units per composition, less than about 9.0 endotoxin units per composition, less than about 8.0 endotoxin units per composition, less than about 7.0 endotoxin units per composition, less than about 6.0 endotoxin units per composition, less than about 5.0 endotoxin units per composition, less than about 4.0 endotoxin units per composition, less than about 3.0 endotoxin units per composition, less than about 2.0 endotoxin units per composition, and/or less than about 1.0 endotoxin units per composition.

In some embodiments, the keratin composition has from about 1.0 to about 20.0 endotoxin units per composition. In some embodiments, the keratin composition has from about 1.0 to about 19.0 endotoxin units per composition, from about 1.0 to about 18.0 endotoxin units per composition, from about 1.0 to about 17.0 endotoxin units per composition, from about 1.0 to about 16.0 endotoxin units per composition, from about 1.0 to about 15.0 endotoxin units per composition, from about 1.0 to about 14.0 endotoxin units per composition, from about 1.0 to about 13.0 endotoxin units per composition, from about 1.0 to about 12.0 endotoxin units per composition, from about 1.0 to about 11.0 endotoxin units per composition, from about 1.0 to about 10.0 endotoxin units per composition, from about 1.0 to about 9.0 endotoxin units per composition, from about 1.0 to about 8.0 endotoxin units per composition, from about 1.0 to about 7.0 endotoxin units per composition, from about 1.0 to about 6.0 endotoxin units per composition, from about 1.0 to about 5.0 endotoxin units per composition, from about 1.0 to about 4.0 endotoxin units per composition, from about 1.0 to about 3.0 endotoxin units per composition, and/or from about 1.0 to about 2.0 endotoxin units per composition.

In some embodiments, the keratin composition comprises a beta-keratin extract, wherein the keratin composition has less than about 400 endotoxin unit per mg of composition. In some embodiments, the keratin composition comprises a beta-keratin network, wherein the keratin composition has less than about 400 endotoxin unit per mg of composition. In some embodiments, the keratin composition has less than about 350 endotoxin units per mg, less than about 300 endotoxin units per mg, less than about 250 endotoxin units per mg, less than about 200 endotoxin units per mg, less than about 200 endotoxin units per mg, less than about 150 endotoxin units per mg, less than about 100 endotoxin units per mg, and/or less than about 50 endotoxin units per mg of composition.

In some embodiments, the keratin composition has from about 1 to about 400 endotoxin units per mg of composition. In some embodiments, the keratin composition has from about 1 to about 350 endotoxin units per mg of composition, from about 1 to about 300 endotoxin units per mg, from about 1 to about 250 endotoxin units per mg, from about 1 to about 200 endotoxin units per mg, from about 1 to about 150 endotoxin units per mg, from about 1 to about 100 endotoxin units per mg, and/or from about 1 to about 50 endotoxin units per mg of composition.

In some embodiments, the keratin composition has less than about 20.0 endotoxin units per mg of composition. In some embodiments, the keratin composition has less than about 19.0 endotoxin units per mg, less than about 18.0 endotoxin units per mg, less than about 17.0 endotoxin units per mg, less than about 16.0 endotoxin units per mg, less than about 15.0 endotoxin units per mg, less than about 14.0 endotoxin units per mg, less than about 13.0 endotoxin units per mg, less than about 12.0 endotoxin units per mg, less than about 11.0 endotoxin units per mg, less than about 10.0 endotoxin units per mg, less than about 9.0 endotoxin units per mg, less than about 8.0 endotoxin units per mg, less than about 7.0 endotoxin units per mg, less than about 6.0 endotoxin units per mg, less than about 5.0 endotoxin units per mg, less than about 4.0 endotoxin units per mg, less than about 3.0 endotoxin units per mg, less than about 2.0 endotoxin units per mg, and/or less than about 1.0 endotoxin units per mg of composition.

In some embodiments, the keratin composition has from about 1.0 to about 20.0 endotoxin units per mg of composition. In some embodiments, the keratin composition has from about 1.0 to about 19.0 endotoxin units per mg, from about 1.0 to about 18.0 endotoxin units per mg, from about 1.0 to about 17.0 endotoxin units per mg, from about 1.0 to about 16.0 endotoxin units per mg, from about 1.0 to about 15.0 endotoxin units per mg, from about 1.0 to about 14.0 endotoxin units per mg, from about 1.0 to about 13.0 endotoxin units per mg, from about 1.0 to about 12.0 endotoxin units per mg, from about 1.0 to about 11.0 endotoxin units per mg, from about 1.0 to about 10.0 endotoxin units per mg, from about 1.0 to about 9.0 endotoxin units per mg, from about 1.0 to about 8.0 endotoxin units per mg, from about 1.0 to about 7.0 endotoxin units per mg, from about 1.0 to about 6.0 endotoxin units per mg, from about 1.0 to about 5.0 endotoxin units per mg, from about 1.0 to about 4.0 endotoxin units per mg, from about 1.0 to about 3.0 endotoxin units per mg, and/or from about 1.0 to about 2.0 endotoxin units per mg of composition.

In some embodiments, the keratin composition comprises a beta-keratin extract, wherein the keratin composition has less than about 400 endotoxin unit per cm². In some embodiments, the keratin composition comprises a beta-keratin network, wherein the keratin composition has less than about 400 endotoxin unit per cm². In some embodiments, the keratin composition has less than about 350 endotoxin units per cm², less than about 300 endotoxin units per cm², less than about 250 endotoxin units per cm², less than about 200 endotoxin units per cm², less than about 200 endotoxin units per cm², less than about 150 endotoxin units per cm², less than about 100 endotoxin units per cm², and/or less than about 50 endotoxin units per cm².

In some embodiments, the keratin composition has from about 1 to about 400 endotoxin units per cm². In some embodiments, the keratin composition has from about 1 to about 350 endotoxin units per cm², from about 1 to about 300 endotoxin units per cm², from about 1 to about 250 endotoxin units per cm², from about 1 to about 200 endotoxin units per cm², from about 1 to about 150 endotoxin units per cm², from about 1 to about 100 endotoxin units per cm², and/or from about 1 to about 50 endotoxin units per cm².

In some embodiments, the keratin composition has less than about 20.0 endotoxin unit per cm². In some embodiments, the keratin composition has less than about 19.0 endotoxin units per cm², less than about 18.0 endotoxin units per cm², less than about 17.0 endotoxin units per cm², less than about 16.0 endotoxin units per cm², less than about 15.0 endotoxin units per cm², less than about 14.0 endotoxin units per cm², less than about 13.0 endotoxin units per cm², less than about 12.0 endotoxin units per cm², less than about 11.0 endotoxin units per cm², less than about 10.0 endotoxin units per cm², less than about 9.0 endotoxin units per cm², less than about 8.0 endotoxin units per cm², less than about 7.0 endotoxin units per cm², less than about 6.0 endotoxin units per cm², less than about 5.0 endotoxin units per cm², less than about 4.0 endotoxin units per cm², less than about 3.0 endotoxin units per cm², less than about 2.0 endotoxin units per cm², and/or less than about 1.0 endotoxin units per cm².

In some embodiments, the keratin composition has from about 1.0 to about 20.0 endotoxin units per cm². In some embodiments, the keratin composition has from about 1.0 to about 19.0 endotoxin units per cm², from about 1.0 to about 18.0 endotoxin units per cm², from about 1.0 to about 17.0 endotoxin units per cm², from about 1.0 to about 16.0 endotoxin units per cm², from about 1.0 to about 15.0 endotoxin units per cm², from about 1.0 to about 14.0 endotoxin units per cm², from about 1.0 to about 13.0 endotoxin units per cm², from about 1.0 to about 12.0 endotoxin units per cm², from about 1.0 to about 11.0 endotoxin units per cm², from about 1.0 to about 10.0 endotoxin units per cm², from about 1.0 to about 9.0 endotoxin units per cm², from about 1.0 to about 8.0 endotoxin units per cm², from about 1.0 to about 7.0 endotoxin units per cm², from about 1.0 to about 6.0 endotoxin units per cm², from about 1.0 to about 5.0 endotoxin units per cm², from about 1.0 to about 4.0 endotoxin units per cm², from about 1.0 to about 3.0 endotoxin units per cm², and/or from about 1.0 to about 2.0 endotoxin units per cm².

In some embodiments, the keratin composition comprises a beta-keratin extract, wherein the keratin composition has less than about 400 endotoxin unit per cm³. In some embodiments, the keratin composition comprises a beta-keratin network, wherein the keratin composition has less than about 400 endotoxin unit per cm³. In some embodiments, the keratin composition has less than about 350 endotoxin units per cm³, less than about 300 endotoxin units per cm³, less than about 250 endotoxin units per cm³, less than about 200 endotoxin units per cm³, less than about 200 endotoxin units per cm³, less than about 150 endotoxin units per cm³, less than about 100 endotoxin units per cm³, and/or less than about 50 endotoxin units per cm³.

In some embodiments, the keratin composition has from about 1 to about 400 endotoxin units per cm³. In some embodiments, the keratin composition has from about 1 to about 350 endotoxin units per cm³, from about 1 to about 300 endotoxin units per cm³, from about 1 to about 250 endotoxin units per cm³, from about 1 to about 200 endotoxin units per cm³, from about 1 to about 150 endotoxin units per cm³, from about 1 to about 100 endotoxin units per cm³, and/or from about 1 to about 50 endotoxin units per cm³.

In some embodiments, the keratin composition has less than about 20.0 endotoxin unit per cm³. In some embodiments, the keratin composition has less than about 19.0 endotoxin units per cm³, less than about 18.0 endotoxin units per cm³, less than about 17.0 endotoxin units per cm³, less than about 16.0 endotoxin units per cm³, less than about 15.0 endotoxin units per cm³, less than about 14.0 endotoxin units per cm³, less than about 13.0 endotoxin units per cm³, less than about 12.0 endotoxin units per cm³, less than about 11.0 endotoxin units per cm³, less than about 10.0 endotoxin units per cm³, less than about 9.0 endotoxin units per cm³, less than about 8.0 endotoxin units per cm³, less than about 7.0 endotoxin units per cm³, less than about 6.0 endotoxin units per cm³, less than about 5.0 endotoxin units per cm³, less than about 4.0 endotoxin units per cm³, less than about 3.0 endotoxin units per cm³, less than about 2.0 endotoxin units per cm³, and/or less than about 1.0 endotoxin units per cm³.

In some embodiments, the keratin composition has from about 1.0 to about 20.0 endotoxin units per cm³. In some embodiments, the keratin composition has from about 1.0 to about 19.0 endotoxin units per cm³, from about 1.0 to about 18.0 endotoxin units per cm³, from about 1.0 to about 17.0 endotoxin units per cm³, from about 1.0 to about 16.0 endotoxin units per cm³, from about 1.0 to about 15.0 endotoxin units per cm³, from about 1.0 to about 14.0 endotoxin units per cm³, from about 1.0 to about 13.0 endotoxin units per cm³, from about 1.0 to about 12.0 endotoxin units per cm³, from about 1.0 to about 11.0 endotoxin units per cm³, from about 1.0 to about 10.0 endotoxin units per cm³, from about 1.0 to about 9.0 endotoxin units per cm³, from about 1.0 to about 8.0 endotoxin units per cm³, from about 1.0 to about 7.0 endotoxin units per cm³, from about 1.0 to about 6.0 endotoxin units per cm³, from about 1.0 to about 5.0 endotoxin units per cm³, from about 1.0 to about 4.0 endotoxin units per cm³, from about 1.0 to about 3.0 endotoxin units per cm³, and/or from about 1.0 to about 2.0 endotoxin units per cm³.

Maintaining a moist wound environment is essential for, among other things, proper wound healing. Permeability of wound care devices, such as keratin compositions, may aid in efficient moisture management of wounds.

Accordingly, in some embodiments, the keratin composition comprises a beta-keratin network, wherein the keratin composition has a moisture vapor transmission rate that is at least about 1000 g/(m²*day) of material per day. In some embodiments, the keratin composition has a moisture vapor transmission rate that is at least about 1500 g/(m²*day) of material per day, at least about 2000 g/(m²*day) of material per day, at least about 2500 g/(m²*day) of material per day, at least about 3000 g/(m²*day) of material per day, at least about 3500 g/(m²*day) of material per day, at least about 4000 g/(m²*day) of material per day, at least about 4500 g/(m²*day) of material per day, at least about 5000 g/(m²*day) of material per day, at least about 5500 g/(m²*day) of material per day, and/or at least about 6000 g/(m²*day) of material per day.

In some embodiments, the keratin composition has a moisture vapor transmission rate that is from about 1000 g/(m²*day) to about 6000 g/(m²*day) of material per day, from about 1500 g/(m²*day) to about 6000/(m²*day) of material per day, from about 2000 g/(m²*day) to about 6000/(m²*day) of material per day, from about 2500 g/(m²*day) to about 6000/(m²*day) of material per day, from about 3000 g/(m²*day) to about 6000/(m²*day) of material per day, from about 3500 g/(m²*day) to about 6000/(m²*day) of material per day, from about 4000 g/(m²*day) to about 6000/(m²*day) of material per day, from about 4500 g/(m²*day) to about 6000/(m²*day) of material per day, from about 5000 g/(m²*day) to about 6000/(m²*day) of material per day, and/or from about 5500 g/(m²*day) to about 6000/(m²*day) of material per day.

In some embodiments, the keratin composition comprises a beta-keratin network, wherein the keratin composition has a thickness of greater than about 0.2 mm. In some embodiments, the keratin composition has a thickness of greater than about 0.3 mm, greater than about 0.4 mm, greater than about 0.5 mm, greater than about 0.6 mm, greater than about 0.7 mm, greater than about 0.8 mm, greater than about 0.9 mm, greater than about 1 mm, greater than about 1.1 mm, greater than about 1.2 mm, greater than about 1.3 mm, greater than about 1.4 mm, and/or greater than about 1.5 mm.

In some embodiments, the keratin composition has a thickness ranging from about 0.2 mm to about 2.0 mm, from about 0.2 mm to about 1.9 mm, from about 0.2 mm to about 1.8 mm, from about 0.2 mm to about 1.7 mm, from about 0.2 mm to about 1.6 mm, from about 0.2 mm to about 1.5 mm, from about 0.2 mm to about 1.4 mm, from about 0.2 mm to about 1.3 mm, from about 0.2 mm to about 1.2 mm, from about 0.2 mm to about 1.1 mm, from about 0.2 mm to about 1.0 mm, from about 0.2 mm to about 0.9 mm, from about 0.2 mm to about 0.8 mm, from about 0.2 mm to about 0.7 mm, from about 0.2 mm to about 0.6 mm, from about 0.2 mm to about 0.5 mm, from about 0.2 mm to about 0.4 mm, and/or from about 0.2 mm to about 0.3 mm.

In some embodiments, the keratin composition has a thickness ranging from about 0.3 mm to 1 mm, from about 0.4 mm to 1 mm, from about 0.5 mm to 1 mm, from about 0.6 mm to 1 mm, from about 0.7 mm to 1 mm, from about 0.8 mm to 1 mm, and/or from about 0.9 mm to 1 mm.

In some embodiments, the keratin composition has a thickness ranging from about 0.4 mm to 0.9 mm, from about 0.6 mm to 0.8 mm, and/or from 0.6 mm to 0.7 mm.

Keramatrix®, a commercially available alpha-keratin containing product, contains less than about 15 mg/cm³ of alpha-keratin monomers, oligomers, and/or filaments. However, higher concentrations of keratin monomers, oligomers, and/or filaments may be advantageous in certain circumstances as it may lead to better physical properties of the composition as well as better performance resulting from greater network formation.

In some embodiments, the keratin composition comprises a beta-keratin extract, wherein the keratin composition comprises at least about 15 mg/cm³ of beta-keratin monomers, oligomers, and/or filaments. In some embodiments, the keratin composition comprises a beta-keratin network, wherein the keratin composition comprises at least about 15 mg/cm³ of beta-keratin monomers, oligomers, and/or filaments.

In some embodiments, the keratin composition comprises a beta-keratin extract, wherein the keratin composition comprises from about 15 mg/cm³ to about 100 mg/cm³ of beta-keratin monomers, oligomers, and/or filaments. In some embodiments, the keratin composition comprises from about 15 mg/cm³ to about 90 mg/cm³ of beta-keratin monomers, oligomers, and/or filaments. In some embodiments, the keratin composition comprises from about 15 mg/cm³ to about 80 mg/cm³, from about 15 mg/cm³ to about 75 mg/cm³, from about 15 mg/cm³ to about 70 mg/cm³, from about 15 mg/cm³ to about 65 mg/cm³, from about 15 mg/cm³ to about 60 mg/cm³, from about 15 mg/cm³ to about 55 mg/cm³, from about 15 mg/cm³ to about 50 mg/cm³, from about 15 mg/cm³ to about 45 mg/cm³, from about 15 mg/cm³ to about 40 mg/cm³, from about 15 mg/cm³ to about 35 mg/cm³, from about 15 mg/cm³ to about 30 mg/cm³, from about 15 mg/cm³ to about 25 mg/cm³, and/or from about 15 mg/cm³ to about 20 mg/cm³ of beta-keratin monomers, oligomers, and/or filaments.

In some embodiments, the keratin composition is in the form of a continuous material. In some embodiments, the continuous material is in a form chosen from a sheet, a gel, a fiber and a foam.

In some embodiments, the keratin composition is in the form of a square, rectangular or cylindrical sheet. In some embodiments, the keratin composition is a square sheet measuring about 1 cm×1 cm. In some embodiments, the keratin composition is a square sheet measuring about 5 cm×5 cm, about 10 cm×10 cm, about 15 cm×15 cm, about 20 cm×20 cm, about 25 cm×25 cm, or about 30 cm×30 cm. In some embodiments, the keratin composition is a cylindrical sheet measuring from about 1 mm to about 30 cm in diameter. In some embodiments, the keratin composition is a cylindrical sheet measuring about 5 mm in diameter, about 6 mm in diameter, about 7 mm in diameter, about 8 mm in diameter, about 9 mm in diameter, about 10 mm in diameter, about 11 mm in diameter, about 12 mm in diameter, about 13 mm in diameter, about 14 mm in diameter, about 15 mm in diameter, about 16 mm in diameter, about 17 mm in diameter, about 18 mm in diameter, about 19 mm in diameter, or about 20 mm in diameter. In some embodiments, the keratin composition is a cylindrical sheet measuring about 2 cm in diameter, about 4 cm in diameter, about 6 cm in diameter, about 8 cm in diameter, about 10 cm in diameter, about 12 cm in diameter, about 14 cm in diameter, about 16 cm in diameter, about 18 cm in diameter, about 20 cm in diameter, about 22 cm in diameter, about 24 cm in diameter, about 26 cm in diameter, about 28 cm in diameter or about 30 cm in diameter. In some embodiments, the sheet is fenestrated, notched, rolled, embossed, folded, or patterned to create an open structure. In some embodiments, the sheet is fenestrated.

In some embodiments, the keratin composition is in the form of a sheet that is a coating on the surface of another material or device. In some embodiments, the keratin composition is coated onto a polyurethane substrate, such as a foam substrate. In some embodiments, the keratin composition is coated onto a backing, such as silicon. In some embodiments, the keratin composition is in the form of a composite material. In some embodiments, the keratin composition is in the form of a layered material.

In some embodiments, the keratin composition is in the form of a hydrogel, a cryogel or a thermogel. In some embodiments, the keratin composition is a hydrogel. In some embodiments, the hydrogel contains greater than 40% water. In some embodiments, the hydrogels are formed by physical or chemical crosslinking, electrospinning, 3D printing, salt or porogen templating, thermogelation and/or cryogelation.

In some embodiments, the keratin composition is a fiber. In some embodiments, the fiber is spun into a nanofibrous mat. In some embodiments, the fiber can be prepared in the form of a suture.

In some embodiments, the keratin composition can be patterned in the form of a 3D architecture. In some embodiments, the keratin composition can be prepared in the form of a mesh. In some embodiments, the keratin composition is in the form of a foam.

In some embodiments, the keratin composition comprises a beta-keratin extract, wherein the keratin composition has less than about 400 endotoxin units per about 1 mL of the composition. In some embodiments, the keratin composition comprises a beta-keratin network, wherein the keratin composition has less than about 400 endotoxin units per about 1 mL of the composition. In some embodiments, the keratin composition has less than about 350 endotoxin units per about 1 mL, less than about 300 endotoxin units per about 1 mL, less than about 250 endotoxin units per about 1 mL, less than about 200 endotoxin units per about 1 mL, less than about 200 endotoxin units per about 1 mL, less than about 150 endotoxin units per about 1 mL, less than about 100 endotoxin units per about 1 mL, and/or less than about 50 endotoxin units per about 1 mL of the composition.

In some embodiments, the keratin composition has from about 0.005 to about 400 endotoxin units per about 1 mL of the composition. In some embodiments, the keratin composition has from about 0.005 to about 350 endotoxin units per about 1 mL, from about 0.005 to about 300 endotoxin units per about 1 mL, from about 0.005 to about 250 endotoxin units per about 1 mL, from about 0.005 to about 200 endotoxin units per about 1 mL, from about 0.005 to about 150 endotoxin units per about 1 mL, from about 0.005 to about 100 endotoxin units per about 1 mL, and/or from about 0.005 to about 50 endotoxin units per about 1 mL of the composition.

In some embodiments, the keratin composition has from about 0.05 to about 400 endotoxin units per about 1 mL of the composition. In some embodiments, the keratin composition has from about 0.05 to about 350 endotoxin units per about 1 mL, from about 0.05 to about 300 endotoxin units per about 1 mL, from about 0.05 to about 250 endotoxin units per about 1 mL, from about 0.05 to about 200 endotoxin units per about 1 mL, from about 0.05 to about 150 endotoxin units per about 1 mL, from about 0.05 to about 100 endotoxin units per about 1 mL, and/or from about 0.05 to about 50 endotoxin units per about 1 mL of the composition.

In some embodiments, the keratin composition has less than about 20 endotoxin units per about 1 mL of the composition. In some embodiments, the keratin composition has less than about 19 endotoxin units per about 1 mL, less than about 18.0 endotoxin units per about 1 mL, less than about 17 endotoxin units per about 1 mL, less than about 16.0 endotoxin units per about 1 mL, less than about 15.0 endotoxin units per about 1 mL, less than about 14.0 endotoxin units per about 1 mL, less than about 13.0 endotoxin units per about 1 mL, less than about 12.0 endotoxin units per about 1 mL, less than about 11.0 endotoxin units per about 1 mL, less than about 10.0 endotoxin units per about 1 mL, less than about 9.0 endotoxin units per about 1 mL, less than about 8.0 endotoxin units per about 1 mL, less than about 7.0 endotoxin units per about 1 mL, less than about 6.0 endotoxin units per about 1 mL, less than about 5.0 endotoxin units per about 1 mL, less than about 4.0 endotoxin units per about 1 mL, less than about 3.0 endotoxin units per about 1 mL, less than about 2.0 endotoxin units per about 1 mL, and/or less than about 1.0 endotoxin unit per about 1 mL of the composition.

In some embodiments, the keratin composition has from about 0.005 to about 20.0 endotoxin units per about 1 mL of the composition. In some embodiments, the keratin composition has from about 0.005 to about 19.0 endotoxin units per about 1 mL, from about 0.005 to about 18.0 endotoxin units per about 1 mL, from about 0.005 to about 17.0 endotoxin units per about 1 mL, from about 0.005 to about 16.0 endotoxin units per about 1 mL, from about 0.005 to about 15.0 endotoxin units per about 1 mL, from about 0.005 to about 14.0 endotoxin units per about 1 mL, from about 0.005 to about 13.0 endotoxin units per about 1 mL, from about 0.005 to about 12.0 endotoxin units per about 1 mL, from about 0.005 to about 11.0 endotoxin units per about 1 mL, from about 0.005 to about 10.0 endotoxin units per about 1 mL, from about 0.005 to about 9.0 endotoxin units per about 1 mL, from about 0.005 to about 8.0 endotoxin units per about 1 mL, from about 0.005 to about 7.0 endotoxin units per about 1 mL, from about 0.005 to about 6.0 endotoxin units per about 1 mL, from about 0.005 to about 5.0 endotoxin units per about 1 mL, from about 0.005 to about 4.0 endotoxin units per about 1 mL, from about 0.005 to about 3.0 endotoxin units per about 1 mL, from about 0.005 to about 2.0 endotoxin units per about 1 mL of the composition, and/or from about 0.005 to about 1.0 endotoxin unit per about 1 mL of composition.

In some embodiments, the keratin composition has from about 0.05 to about 20.0 endotoxin units per about 1 mL of the composition. In some embodiments, the keratin composition has from about 0.05 to about 19.0 endotoxin units per about 1 mL, from about 0.05 to about 18.0 endotoxin units per about 1 mL, from about 0.05 to about 17.0 endotoxin units per about 1 mL, from about 0.05 to about 16.0 endotoxin units per about 1 mL, from about 0.05 to about 15.0 endotoxin units per about 1 mL, from about 0.05 to about 14.0 endotoxin units per about 1 mL, from about 0.05 to about 13.0 endotoxin units per about 1 mL, from about 0.05 to about 12.0 endotoxin units per about 1 mL, from about 0.05 to about 11.0 endotoxin units per about 1 mL, from about 0.05 to about 10.0 endotoxin units per about 1 mL, from about 0.05 to about 9.0 endotoxin units per about 1 mL, from about 0.05 to about 8.0 endotoxin units per about 1 mL, from about 0.05 to about 7.0 endotoxin units per about 1 mL, from about 0.05 to about 6.0 endotoxin units per about 1 mL, from about 0.05 to about 5.0 endotoxin units per about 1 mL, from about 0.05 to about 4.0 endotoxin units per about 1 mL, from about 0.05 to about 3.0 endotoxin units per about 1 mL, from about 0.05 to about 2.0 endotoxin units per about 1 mL, and/or from about 0.05 to about 1.0 endotoxin unit per about 1 mL of the composition.

In some embodiments, the keratin composition is in a form chosen from a liquid, a dispersion and particles, such as a powder. In some embodiments, the liquid is a keratin sprayable liquid. In some embodiments, the dispersion is a slurry, suspension, cream or colloid. In some embodiments, the dispersion is a suspension.

In some embodiments, the keratin composition is in the form of particles. In some embodiments, the particles have an average particle size range of from about 8 nm to about 10 mm. In some embodiments, the average particle size is from about 5 nm to about 2 mm. In some embodiments, the keratin composition is in the form of a powder.

In some embodiments, the particles are nanoparticles. In some embodiments, the nanoparticles are chosen from zero-dimensional nanomaterials, nanocapsules and nanospheres. Nanocapsule refers to a nanostructure made of a shell, which can be a polymeric membrane for example, and a core space where a desired substance(s) can be loaded. Nanospheres refer to nanostructures where the entire mass is solid and consists of spherical polymeric matrices which can be used as a carrier.

In some embodiments, the keratin composition is a sheet having less than about 25% residual moisture by Karl Fisher titration. In some embodiments, the sheet has less than about 20% residual moisture. Residual moisture can be measured, for example, by placing a sample into a glass vial and stored in a desiccator until use. The weight is then recorded using an analytical balance. A blank sample vial is prepared and treated in the same manner as the sample. The sample and blank are heated to 100° C. and a needle is used to pierce the cap of each vial. The needle washes dry nitrogen across the sample and through a transfer line into a titrator such as a Mettler-Toledo, C30 Coulometric Titrator. The titration continues until all the moisture transfer is complete. The blank value is then subtracted from the test sample to ensure that the system suitability control is within 10% of the true value of the standard. The loss in sample mass, calculated by subtracting the final sample weight from the initial sample weight, is attributed to the residual moisture content of the sample.

In some embodiments, the keratin composition is free of crosslinking agents. In some embodiments, the keratin composition is free of H₂O₂. In some embodiments, the keratin composition is free of a catalyst, promoter or initiator.

In some embodiments, the keratin composition is free of cysteine modified keratin. In some embodiments, the cysteine modified keratin is a partially carboxylated keratin or partially carboxymethylated keratin.

In some embodiments, the keratin composition is free of impurities. In some embodiments, the impurities include, for example, detergents such as sodium dodecyl sulfate, chaotropic compounds such as urea, cells, cellular material, and reducing agents such as thioglycolic acid.

In some embodiments, the keratin composition further comprises at least one additive. In some embodiments, the at least one additive is chosen from a protein, a carbohydrate, a polymer, a plasticizer, an antimicrobial agent, an anti-scarring agent, an anti-inflammatory agent, an angiogenic agent, a cell, a bulking agent, a stabilizer, a proteoglycan and a polysaccharide. In some embodiments, the keratin composition comprises at least one plasticizer and at least one polymer.

In some embodiments, the protein is chosen from an alpha-keratin monomer, oligomer and/or filament, a keratin-associated protein, a collagen, an elastin, a glycoprotein, a growth factor, a cytokine, silk, laminin, an antibody, an antimicrobial peptide, an integrin, an enzyme, an interleukin and a proteoglycan. In some embodiments, the keratin composition further comprises alpha-keratin monomers, oligomers and/or filaments.

In some embodiments, the carbohydrate is a monosaccharide, a disaccharide, an oligosaccharide, or a polysaccharide. In some embodiments, the polysaccharide is a glycosaminoglycan. In some embodiments, the glycosaminoglycan is hyaluronic acid.

In some embodiments, the plasticizer is chosen from glycerol, propylene glycol, triethyl citrate, diethyl phthalate, acetylated monoglycerides, and oils. In some embodiments, the keratin composition comprises a plasticizer at a concentration from about 0-19% w/w of the composition. In some embodiments, the plasticizer is at a concentration of about 20-59% w/w of the composition. In some embodiments, the plasticizer is at a concentration greater than about 60% w/w of the composition.

In some embodiments, the plasticizer is glycerol. In some embodiments, the glycerol is at a concentration of about 0-19% w/w of the composition. In some embodiments, the glycerol is at a concentration of about 20-59% w/w of the composition. In some embodiments, the glycerol is at a concentration of greater than about 60% w/w of the composition.

In some embodiments, the polymer is chosen from polyethylene glycol, polycaprolactone, polyurethane, polyvinyl alcohol, polylactic acid, polyhexamethylene biguanide, polylactic-co-glycolic acid, or poly(N-isopropylacrylamide). In some embodiments, the keratin composition comprises a polymer at a concentration of about 0-49% w/w of the composition. In some embodiments, the polymer is at a concentration of about 50-69% w/w of the composition. In some embodiments, the polymer is at a concentration of greater than about 70% w/w of the composition.

In some embodiments, the polymer is polyethylene glycol. In some embodiments, the polyethylene glycol is at a concentration of about 0-49% w/w of the composition. In some embodiments, the polyethylene glycol is at a concentration of about 50-69% w/w of the composition. In some embodiments, the polyethylene glycol is at a concentration of greater than about 70% w/w of the composition.

In some embodiments, the polyethylene glycol is a low molecular weight polyethylene glycol (<5,000 g/mol). In some embodiments, the polyethylene glycol is a medium molecular weight polyethylene glycol (5000-19,999 g/mol). In some embodiments, the polyethylene glycol is a high molecular weight polyethylene glycol (≥20,000 g/mol, also known as polyethylene oxide). In some embodiments, the keratin composition comprises more than one size of polyethylene glycol.

In some embodiments, the keratin composition comprises at least one plasticizer at a concentration of about 0-19% w/w of the composition and at least one polymer at a concentration of about 0-49% w/w of the composition. In some embodiments, the keratin composition comprises at least one plasticizer at a concentration of about 0-19% w/w of the composition and at least one polymer at a concentration of about 50-69% w/w of the composition. In some embodiments, the keratin composition comprises at least one plasticizer at a concentration of about 0-19% w/w of the composition and at least one polymer at a concentration of greater than about 70% of the composition.

In some embodiments, the keratin composition comprises at least one plasticizer at a concentration of about 20-59% w/w of the composition and at least one polymer at a concentration of about 0-49% w/w of the composition. In some embodiments, the keratin composition comprises at least one plasticizer at a concentration of about 20-59% w/w of the composition and at least one polymer at a concentration of about 50-69% w/w of the composition. In some embodiments, the keratin composition comprises at least one plasticizer at a concentration of about 20-59% w/w of the composition and at least one polymer at a concentration of greater than about 70% of the composition.

In some embodiments, the keratin composition comprises at least one plasticizer at a concentration of greater than about 60% w/w of the composition and at least one polymer at a concentration of about 0-49% w/w of the composition. In some embodiments, the keratin composition comprises at least one plasticizer at a concentration of greater than about 60% of the composition and at least one polymer at a concentration of about 50-69% w/w of the composition. In some embodiments, the keratin composition comprises at least one plasticizer at a concentration of greater than about 60% of the composition and at least one polymer at a concentration of greater than about 70% of the composition.

In some embodiments, the keratin composition does not comprise a plasticizer. In some embodiments, the keratin composition does not comprise glycerol. In some embodiments, the keratin composition does not comprise a polymer. In some embodiments, the keratin composition does not comprise polyethylene glycol.

In some embodiments, the keratin composition interacts with a cell. In some embodiments, the keratin composition facilitates a moist environment for a cell. In some embodiments, the keratin composition allows for cells to attach to the composition. In some embodiments, the keratin composition allows for cell proliferation. Cell proliferation can be measured, for example, by the Bromodeoxyuridine (BrdU) assay and/or the WS1 human fibroblast assay as described in Example 10.

In some embodiments, the keratin compositions are sterile. In some embodiments, the keratin compositions are single-use. In some embodiments, the keratin composition can be kitted, embossed, labelled and/or perforated.

In some embodiments, the keratin composition is capable of absorbing fluids. In some embodiments, the keratin composition is capable of forming a gel when in contact with a liquid. In some embodiments, the fluid is chosen from water, saline or a wound exudate.

In some embodiments, the keratin compositions are pharmaceutical keratin compositions.

In some embodiments, the keratin composition is prepared according to any of the following methods.

### VI. Exemplary Embodiments of Methods of Preparing Compositions

In one aspect, methods of preparing keratin compositions are disclosed herein. In some embodiments, the method comprises preparing a keratin composition comprising a protein extract of any of the foregoing embodiments, e.g., Section III. In some embodiments, the method comprises preparing a keratin composition comprising combining a protein extract of any of the foregoing embodiments with at least one additive to give a keratin composition.

In some embodiments, the additive is combined with the protein extract at about 0-19% w/w of the extract. In some embodiments, the additive is combined with the protein extract at about 20-59% w/w of the extract. In some embodiments, the additive is combined with the protein extract at greater than about 60% w/w of the extract.

In some embodiments, the method further comprises mixing the protein extract with a solvent prior to combining with the at least one additive. In one embodiment, the solvent comprises water, a buffer, ethanol, methanol, poly(vinyl alcohol), formic acid, isopropanol, hexane, and/or hexafluoroisopropanol (HFIP). In one embodiment, the solvent comprises water. In one embodiment, the solvent comprises a buffer. In some embodiments, the buffer is PBS, citrate, HEPES, acetate. In some embodiments, the buffer is PBS with a pH at about 7 or at about 7.9. In some embodiments, the solvent does not comprise an acid or base. In some embodiments, the solvent does not comprise NaOH, HCL, and/or formic acid.

In some embodiments, the method further comprises casting the keratin composition onto a surface. In some embodiments, the surface is a mold. In some embodiments, the surface is in the shape of a circle. In some embodiments, the shape is a rectangle, such as a square. In some embodiments, the mold has a thickness of greater than about 0.2 mm. In some embodiments, the mold has a thickness of greater than about 0.3 mm, greater than about 0.4 mm, greater than about 0.5 mm, greater than about 0.6 mm, greater than about 0.7 mm, greater than about 0.8 mm, greater than about 0.9 mm, greater than about 1 mm, greater than about 1.1 mm, greater than about 1.2 mm, greater than about 1.3 mm, greater than about 1.4 mm, greater than about 1.5 mm, greater than about 1.6 mm, greater than about 1.7 mm, greater than about 1.8 mm, greater than about 1.5 mm, greater than about 1.9 mm and/or greater than about 2.0 mm.

In some embodiments, the method further comprises freezing the keratin composition. In some embodiments, the composition is frozen by freezing in a freezer or with liquid nitrogen.

In some embodiments, the method further comprises dehydrating the keratin composition. In some embodiments, the dehydrating is accomplished by heating the composition in an oven, allowing the composition to air dry or drying at a specific humidity. In some embodiments, the freezing and dehydrating is accomplished by lyophilization. In some embodiments, the freezing and dehydrating steps comprise adding an additive comprising a porogen, such as a salt of a specific size, a cryoprotectant, and/or glycerol.

In some embodiments, the method further comprises cutting and/or milling the keratin composition. In some embodiments, the keratin composition that is cut and/milled is a continuous material such as, for example, a sheet.

In some embodiments, the method further comprises spinning the keratin composition into a keratin fiber. In some embodiments, the spinning is accomplished by wet spinning or electrospinning. In some embodiments, the method further comprises fabricating the keratin fibers into a sheet.

In some embodiments, the method further comprises crosslinking the keratin composition. In some embodiments, the crosslinking is chemical and/or physical. In some embodiments, the crosslinking is a combination of chemical and physical crosslinking. In some embodiments, the crosslinking is achieved by chemical means, physical means, enzymatic means, or a combination thereof. In some embodiments, the chemical crosslinking is achieved through chemical reaction of complementary groups, crosslinking with high energy radiation, enzymatic crosslinking, free radical polymerization, crosslinking with chemical crosslinkers, photo crosslinking and/or UV crosslinking.

In some embodiments, the crosslinking does not involve the use of a crosslinking agent. In some embodiments, the crosslinking does not involve the use of a catalyst, promoter or initiator. In some embodiments, the method does not comprise modifying the cysteine residues in the protein extract prior to the crosslinking.

In some embodiments, the at least one additive is chosen from a protein, a carbohydrate, a polymer, a plasticizer, an antimicrobial agent, an anti-scarring agent, an anti-inflammatory agent, a pro-inflammatory agent, an angiogenic agent, a cell, a bulking agent, a stabilizer, a proteoglycan and a polysaccharide. In some embodiments, the keratin composition comprises at least one plasticizer and at least one polymer.

In some embodiments, the protein is chosen from an alpha-keratin monomer, oligomer and/or filament, a keratin-associated protein, a collagen, an elastin, a glycoprotein, a growth factor, a cytokine, silk, laminin, an antibody, an antimicrobial peptide, an integrin, an enzyme, an interleukin and a proteoglycan. In some embodiments, the keratin composition further comprises alpha-keratin monomers, oligomers and/or filament.

In some embodiments, the carbohydrate is a monosaccharide, a disaccharide, an oligosaccharide, or a polysaccharide. In some embodiments, the polysaccharide is a glycosaminoglycan. In some embodiments, the glycosaminoglycan is hyaluronic acid.

In some embodiments, the plasticizer is chosen from glycerol, propylene glycol, triethyl citrate, diethyl phthalate, acetylated monoglycerides, and oils. In some embodiments, the plasticizer is combined with the protein extract at about 0-19% w/w of the extract. In some embodiments, the plasticizer is combined with the protein extract at about 20-59% w/w of the extract. In some embodiments, the plasticizer is combined with the protein extract at greater than about 60% w/w of the extract. In some embodiments, the plasticizer is glycerol.

In some embodiments, the polymer is chosen from polyethylene glycol, polycaprolactone, polyurethane, polyvinyl alcohol, polylactic acid, polyhexamethylene biguanide, polylactic-co-glycolic acid, or poly(N-isopropylacrylamide). In some embodiments, the polymer is combined with the protein extract at about 0-49% w/w of the composition. In some embodiments, the polymer is combined with the protein extract at about 50-69% w/w of the composition. In some embodiments, the polymer is combined with the protein extract at greater than about 70% w/w of the composition. In some embodiments, the plasticizer is polyethylene glycol.

In some embodiments, the polyethylene glycol is a low molecular weight polyethylene glycol. In some embodiments, the polyethylene glycol is a medium molecular weight polyethylene glycol. In some embodiments, the polyethylene glycol is a high molecular weight polyethylene glycol (≤20,000 g/mol, also known as polyethylene oxide). In some embodiments, the keratin composition comprises more than one size of polyethylene glycol.

In some embodiments, at least one plasticizer is combined with the protein extract at about 0-19% w/w of the extract and at least one polymer is combined with the protein extract at about 0-49% w/w of the extract. In some embodiments, at least one plasticizer is combined with the protein extract at about 0-19% w/w of the extract and at least one polymer is combined with the protein extract at about 50-69% w/w of the extract. In some embodiments, at least one plasticizer is combined with the protein extract at about 0-19% w/w of the extract and at least one polymer is combined with the protein extract at greater than about 70% of the extract.

In some embodiments, the keratin composition comprises at least one plasticizer in about 20-59% w/w of the composition and at least one polymer in about 0-49% w/w of the composition. In some embodiments, the keratin composition comprises at least one plasticizer in about 20-59% w/w of the composition and at least one polymer in about 50-69% w/w of the composition. In some embodiments, the keratin composition comprises at least one plasticizer in about 20-59% w/w of the composition and at least one polymer in greater than about 70% of the composition.

In some embodiments, at least one plasticizer is combined with the protein extract at greater than about 60% w/w of the extract and at least one polymer is combined with the protein extract at about 0-49% w/w of the extract. In some embodiments, at least one plasticizer is combined with the protein extract at greater than about 60% of the extract and at least one polymer is combined with the protein extract at about 50-69% w/w of the extract. In some embodiments, at least one plasticizer is combined with the protein extract at greater than about 60% of the extract and at least one polymer is combined with the protein extract at greater than about 70% of the extract.

In some embodiments, the keratin composition does not comprise a plasticizer. In some embodiments, the keratin composition does not comprise glycerol. In some embodiments, the keratin composition does not comprise a polymer. In some embodiments, the keratin composition does not comprise polyethylene glycol.

In some embodiments, a keratin composition is prepared according to any of the methods above.

### VII. Exemplary Embodiments of Methods of Use and Kits

In one aspect, methods of using keratin extracts and keratin compositions, as well as kits comprising keratin extracts and compositions, are disclosed herein.

In some embodiments, disclosed herein is a method of treating a wound. In some embodiments, the method of treating a wound of a subject comprises administering to the wound the keratin composition according to any of the foregoing embodiments or a protein extract according to any of the foregoing embodiments. In some embodiments, the treating comprises healing the wound, closing the wound and/or reducing signs of scars.

In some embodiments, disclosed herein is a method of managing a wound of a subject comprising covering a wound, absorbing exudate from a wound, and/or maintaining appropriate moisture balance within a wound. In some embodiments, the method of managing a wound of a subject comprises covering a wound, absorbing exudate from a wound, and/or maintaining appropriate moisture balance within a wound with the keratin composition according to any of the foregoing embodiments or a protein extract according to any of the foregoing embodiments. In some embodiments, the method of managing a wound of a subject comprises covering a wound. In some embodiments, the method of managing a wound of a subject comprises absorbing exudate from a wound. In some embodiments, the method of managing a wound of a subject comprises maintaining appropriate moisture balance within a wound. In some embodiments, the method of managing a wound of a subject comprises covering a wound, absorbing exudate from a wound, and maintaining appropriate moisture balance within a wound.

In some embodiments, the wound is an acute wound. In some embodiments, the wound is a chronic wound. In some embodiments, the wound is an abrasion, laceration, skin tear, tunneled wound, incision, draining wound, ulcer and/or burn. In some embodiments, the wound is a surgical wound including acute surgical wounds, donor sites or grafts, podiatric wounds, post-Moh's surgery, post-laser surgery, and/or wound dehiscence. In some embodiments, the burn is a first-, second- or third-degree burn. In some embodiments, the burn is a partial-thickness burn. In some embodiments, the burn is chosen from a friction burn, a cold burn, a thermal burn, a radiation burn, a chemical burn and an electrical burn. In some embodiments, the radiation burn is a sunburn. In some embodiments, the ulcer is chosen from a pressure ulcer, a venous ulcer, a diabetic ulcer, an ulcer caused by mixed vascular etiologies and a chronic vascular ulcer.

In some embodiments, the wound is a wound of the dermis, soft tissue, connective tissue, eye, nerve, gums, dura, or bones. In some embodiments, the wound is a partial or full thickness wound.

In some embodiments, the method of treating a wound comprises treating dry, light, moderately or heavily exudating partial and/or full thickness wounds.

In some embodiments, disclosed herein is a method for protecting a tissue of a subject. In some embodiments, the method for protecting a tissue of a subject comprises administering the keratin composition according to any of the foregoing embodiments or a protein extract according to any of the foregoing embodiments to the tissue. In some embodiments, the tissue is chosen from the dermis, soft tissue, connective tissue, eye, nerve, gums, dura and bones.

In some embodiments, in any of the above embodiments, the keratin composition or protein extract is applied to the surface of the wound or the tissue. In some embodiments, the keratin composition applied to the edges of a wound, for example, for treating wound dehiscence.

In some embodiments, in any of the above embodiments, the protein extract is dehydrated.

In some embodiments, the method further comprises sterilizing the keratin composition or protein extract. In some embodiments, the sterilization comprises electron beam radiation, ethylene oxide, autoclaving, or Gamma radiation.

In some embodiments, the keratin compositions are designed for single-use. In some embodiments, the keratin composition can be kitted, embossed, labelled and/or perforated. In some embodiments, the keratin compositions or protein extracts are designed for use as acute and chronic wound treatment devices. In some embodiments, the devices are designed for dry to heavily exuding wounds.

In some embodiments, disclosed herein is a method for delivering a pharmaceutically active agent to a subject in need thereof. In some embodiments, the method comprises administering to the subject in need thereof the keratin composition according to any of the foregoing embodiments, wherein the keratin composition further comprises the pharmaceutically active agent.

In some embodiments, the pharmaceutically active agent is chosen from a protein, an antimicrobial agent, an anti-scarring agent, an anti-inflammatory agent, a pro-inflammatory agent, a small molecule, an angiogenic agent, a gene therapy and a cell. In some embodiments, the protein is chosen from an alpha-keratin monomer, oligomer and/or filament, a keratin-associated protein, a collagen, an elastin, a glycoprotein, a growth factor, a cytokine silk, laminin, an antibody, an antimicrobial peptide, an integrin, an enzyme, an interleukin and a proteoglycan. In some embodiments, the antimicrobial agent is chosen from a small molecule, antimicrobial peptide, antimicrobial polymers such as polyhexamethylene biguanide (PHMB), anti-fungal agent, or silver. In some embodiments, the anti-scarring agent is chosen from a protein, such as basic fibroblast growth factor (bFGF). In some embodiments, the anti-inflammatory agent is chosen from a small molecule drug, anti-inflammatory cytokine, RNA-based drug, or steroid. In some embodiments, the growth factor is chosen from epidermal growth factor (EGF), fibroblast growth factor (FGF), platelet-derived growth factor (PDGF), transforming growth factor-beta (TGF-β), and vascular endothelial growth factor (VEGF). In some embodiments, the cell is chosen from keratinocytes, platelets, fibroblasts, mesenchymal stromal cells, adipose derived stromal cells, epithelial cells, engineered cells. In some embodiments, the pro-inflammatory agent is chosen from IFN-γ, IL-6, IL-1 and TNF-α. In some embodiments, the angiogenic factor is chosen from VEGF, FGF and angiopoietin. In some embodiments, the cytokine is chosen from IL-4, IL-6, IL-1 and TGF-β. In some embodiments, the gene therapy is chosen from CRISPR-Cas9, CAR-T, a viral vector, genetic material, TALENs, zinc finger nucleases and stem cells.

In one aspect, disclosed herein is a kit comprising a protein extract and/or a keratin composition. In some embodiments, the kit comprises a protein extract according to any one of the foregoing embodiments. In some embodiments, the kit comprises a keratin composition according to any of the foregoing embodiments. In some embodiments, the kit comprises both a protein extract according to any one of the foregoing embodiments and a keratin composition according to any of the foregoing embodiments.

In some embodiments, the kit further comprises a syringe. In some embodiments, the syringe contains the keratin composition. In some embodiments, the kit comprises a syringe and a tube or vial that contains the keratin composition. In some embodiments, the kit contains a tube or vial containing the keratin composition without a syringe. In some embodiments, the kit further comprises a reconstitution medium such as water for injection (WFI) or sterile saline.

In some embodiments, the kit further comprises sutures. In some embodiments, the kit further comprises a wound dressing such as gauze, an adhesive material, a non-adhesive material, an antimicrobial agent, a debriding agent, or saline.

In some embodiments, the kit is single-use. In some embodiments, the keratin composition can be kitted, embossed, labelled and/or perforated. In some embodiments, the kit is multi-use.

### EXAMPLES

### Example 1. Washing Methods

In general, keratin is obtained from a keratin source by a series of steps: (1) washing, (2) defatting, and (3) extracting. Washing is typically performed to remove debris, bioburden, and other contaminants. Defatting is useful to remove lipids from the source material. Extraction isolates keratin monomers, oligomers, and/or filaments from the source material (such as from reptiles and chickens).

The present inventors discovered that feathers may have a high level of endotoxins, which can be undesirable in medical devices and other clinical applications. For example, in order to meet FDA requirements for pyrogenicity, medical device products must contain ≤20 endotoxin units (“EU”) per device. Indeed, to date there are no beta-keratin containing biomaterials or other products that are approved or cleared for use by the FDA. In order to address this issue, several methods of washing were investigated as outlined below. Certain methods below include sterilization of the washed feathers.

Washing Method 1: Wet unprocessed white chicken feathers were soaked in a 1.0% v/v triton solution in a wash basin at a ratio of 1 L of unprocessed chicken feathers per 10 L of wash solution for 20 minutes. Manual removal of debris was completed by sifting by hand and removing exogenous material, chicken tissue other than feather and environmental debris. Feathers were then filtered using a mesh and rinsed with water. Feathers were dried at 100° C. for 16-24 hrs.

Washing Method 2a: In this method, the dried feathers from Wash Method 1 were taken and subjected to a second wash step. More specifically, the dried feathers were agitated at a ratio of 10 g of feathers per L of 1% v/v triton solution. A total of three 15 min agitation cycles were completed. After each cycle the feather solution was strained, and the feathers were rinsed with water before proceeding to the next wash cycle. The feathers were dried at 100° C. for 16-24 hrs.

Washing Method 2b: In this method, the dried feathers from Wash Method 1 were subjected to a different second wash step. More specifically, the dried feathers were soaked in a 1% v/v triton solution at a ratio of 10 g/L for 0-30 minutes. The feathers were then strained and subjected to manual or automated agitation. The feathers were then rinsed with water and the cycle was repeated two additional times, followed by drying at 100° C. for 16-24 hrs.

Washing Method 2c: In this method, the dried feathers from Wash Method 1 were subjected to yet another second wash step. More specifically, the dried feathers were agitated with a 1% v/v triton in a ratio of 50 g feathers to 4 L of solution. A total of three cycles was performed, with straining and water rinsing in between each cycle. After 3 cycles, the feathers were rinsed while agitating the feathers using 4 L of water per 50 g feathers for fifteen cycles. After washing, debris was removed, and the feathers were rinsed with water. The feathers were then dried at 100° C. for 16-24 hours.

Washing Method 3: 1 L of unprocessed chicken feathers was soaked with 10 L of a 0.5% w/v SDS solution in a static bath for 20 minutes. Debris was removed and the feathers were strained and rinsed with water several times. The feathers were dried at 100° C. for 16-24 hours. In some instances, the feathers were cut and ground after washing.

Washing Method 4: Unprocessed chicken feathers were washed according to Wash Method 3. After drying as described in Method 3, the feathers were then washed again with 0.5% w/v SDS at a ratio of 4 L of SDS solution to 50 g of feathers. The feathers were agitated with SDS solution for 1 cycle then agitated in 4 L of water to 50 g feathers in water for 15 cycles. The feathers were then dried at 100° C. for 16-24 hours.

Washing Method 5: Unprocessed chicken feathers were washed according to Wash Method 4. After drying as described in Method 4, the feathers were then sterilized at 180-250° C. for 1-4 hours. More specifically, the feathers were placed on a tray at 25-50±5 g of feathers per tray, wet with 0.5-1.5 L of water, and covered with aluminum foil. The feathers were then dried at 180-250° C. for 1-4 hours. This cycle was repeated 1-3 times.

Washing Method 6: Unprocessed chicken feathers were washed according to Wash Method 4. After drying as described in Method 4, the feathers were then sterilized using steam at 180-250° C. for 1-6 hours. More specifically, the feathers were placed on a mesh tray at 25-50±5 g of feathers per tray and placed in a pan of water containing 0.5-1.5 L of water. The tray was then covered in aluminum foil. The feathers were then dried at 180-250° C. for 1-6 hours. This cycle was repeated 1-3 times.

### Example 2. Endotoxin Levels in Washed Feathers

In order to determine the level of endotoxins in the washed feathers, the feathers were subjected to a kinetic chromogenic LAL assay. This in vitro assay is used to detect and quantify bacterial endotoxin, which are a component of the cell wall of gram-negative bacteria. Testing was performed according to Thermo Scientific Pierce™ Chromogenic Endotoxin Quant Kit protocol.

The LAL assay uses amebocyte lysates derived from blood of the horseshoe crab to quantify endotoxin in samples. Endotoxin interactions with amebocyte lysate result in a series of enzymatic reactions triggering the activation of Factor C, Factor B, and pro-clotting enzyme. The activated enzyme catalyzes the release of p-nitroaniline (pNA) from the colorless chromogenic substrate creating a yellow color. The pNA is then measured at 405 nm. Endotoxin concentration is subsequently correlated to absorbance at 405 nm. This relationship is linear from 0.1-1.0 EU/mL and from 0.01-0.1 EU/mL. The endotoxin concentration of test samples can be determined using a standard calibration curve.

Dried feathers were prepared for testing by immersing them (200-400 mg) in endotoxin free water (50 mL) and incubating them at 37-40° C. for no less than 60 minutes with shaking on a shaker plate. An aliquot of resultant fluid was then taken for testing following the kit protocol described above. The results (given in EU/mL) were normalized to the mass of the test article (EU/mg of feathers). The table below shows the reduction in endotoxin levels of certain of the washes listed above.

TABLE 1

| | Endotoxin Levels (EU/mg of | |
|---|---|---|
| Washing Method | dried washed feathers) | |
| Feathers before washing | 11.87 and 18.35 | EU/mg (n = 2) |
| Feathers after Washing Method 1 | 0.2-15.60 | EU/mg (n = 8) |
| Feathers after Washing Method 2a | 0.03 and 0.09 | EU/mg (n = 2) |
| Feathers after Washing Method 2b | 0.002-0.2 | EU/mg (n = 7) |
| Feathers after Washing Method 2c | 0.00004-0.008 | EU/mg (n = 12) |
| Feathers after Washing Method 3 | 3.25-114 | EU/mg (n = 14) |
| Feathers after Washing Method 4 | 0.01-0.10 | EU/mg (n = 5) |
| Feathers after Washing Method 5 | <LOD-0.038 | EU/mg (n = 22) |
| Feathers after Washing Method 6 | <LOD-0.1 | EU/mg (n = 36) |

<LOD means lower than the limit of detection (i.e., less than 0.01 EU/mL).

### Example 3. Extraction Processes to Provide a Keratin Extract

Numerous extractions were performed to find a method that would give a stable keratin extract that could be easily stored and/or could be readily dissolved in a neutral or near neutral solution. After washing, and prior to each of the extraction processes below, the feathers were subjected to a lipid removal step (i.e., defatted) using 95% EtOH for 4 hours in a static bath. More specifically, the feathers were added to the ethanol solution at a ratio of 100 g feathers to 1 L of solution. The material was then filtered, and serial washed with water to separate the feathers from the liquid. The feathers were dried at 100° C. for 16-24 hours and subjected to the reductive extraction processes below to provide keratin extracts in both solution form (e.g., in the extraction solution and/or after dialysis) and in solid form (e.g., after lyophilization).

Extraction Process 1a: White chicken feathers were washed according to Wash Method 3 and then defatted and dried according to the procedures described above. The resultant feathers were then subjected to a reductive extraction in 8 M urea at pH 9 with 200 mM Tris, 3 mM EDTA, and 125 mM 2-mercaptoethanol (7.5 g feathers/100 mL extraction buffer) at 60° C. for 2 hours. Feathers were added to a beaker containing the extraction buffer and the solution was stirred at 150-350 rpm throughout the 2-hour extraction period. The resulting solution was filtered through a 100 μm filter and centrifuged at 4000 rpm for 15 minutes at room temperature, resulting in a yellow solution. The supernatant was then dialyzed against water for 2 days using dialysis tubing with a molecular weight cutoff (MWCO) of 3.5 kDa. The resulting dense, white hydrogel was lyophilized against water for 2 days and then homogenized to produce a dense powder. Yields ranged from 9-74%. Yield was determined by comparing the mass of the lyophilized protein extract to the initial mass of dried feathers extracted.

Extraction process 1b: White chicken feathers were washed according to Wash Method 3 and then defatted and dried according to the procedures described above. The feathers were then subjected to a reductive extraction in 8 M urea at pH 9 with 200 mM Tris, 3 mM EDTA, and 125 mM 2-mercaptoethanol (7.5 g feathers/100 mL extraction buffer) at 60° C. for 2 hours. Feathers were added to a beaker containing the extraction buffer and the solution was stirred at 150-350 rpm throughout the 2-hour extraction period resulting in a pale yellow solution. The resulting solution was filtered through a 100 μm filter and centrifuged at 4000 rpm for 15 minutes at room temperature. Prior to dialysis, the extraction solution was diluted ˜1:7 in extraction buffer and loaded into dialysis tubing with a MWCO of 3.5 kDa and dialyzed against water for 3-5 days. The resulting cloudy solution was then lyophilized for 3-5 days to produce a powdery white material. Yields ranged from 10-20%.

Extraction Process 2: White chicken feathers were washed according to Wash Method 3 and then defatted and dried according to the procedures described above. The feathers were then subjected to a reductive extraction in 8 M urea at pH 10 with 200 mM Tris, 3 mM EDTA, and 1.3 M thioglycolic acid (15 g feathers/1000 mL extraction buffer) at 60° C.-70° C. for 2 hours. The light pink extraction solution was filtered through a 100 μm filter, loaded into dialysis tubing with a MWCO of 3.5 kDa and dialyzed against water for 3-5 days. The resulting cloudy solution was lyophilized for 3-5 days. The lyophilized protein formed a dense off-white material and was then optionally ground into a fluffy off-white powder. Yields ranged from 30-40%.

Extraction Process 3: White chicken feathers were washed according to Wash Method 3 and then defatted and dried according to the procedures described above. The feathers were then subjected to a reductive extraction in 5M urea at pH 7 with 2.5 M thiourea and 5% w/v sodium metabisulfite (13.3 g feathers/200 mL extraction buffer) at 50° C. for 5 hours. The resulting solution was filtered through a 100 μm filter and centrifuged at 4000 rpm for 15 minutes at room temperature, and the supernatant dialyzed against water for 3 days using dialysis tubing with a MWCO of 3.5 kDa. Dialysis produced a clear solution with disperse aggregates, or a dense white hydrogel, and was subsequently lyophilized to a white dense material. Yields ranged from 5-41%.

Extraction Process 4: White chicken feathers were washed according to Wash Method 3 and then defatted and dried according to the procedures described above. The feathers were then subjected to a reductive extraction in 0.45 M urea at pH 9 with 0.5 M thioglycolic acid, 0.05 M SDS, 5% w/v sodium metabisulfite, and 0.004 M Tris (13.3 g feathers/200 mL extraction buffer) at 50° C. for 2 hours. The resulting slightly purple solution was filtered through a 100 μm filter and centrifuged at 4000 rpm for 15 minutes at room temperature, and the supernatant dialyzed against water for 3 days using dialysis tubing with a MWCO of 3.5 kDa. The resulting cloudy solution was then lyophilized to form a dense white material. Yields ranged from 4-11%.

Extraction Process 5: White chicken feathers were washed according to Wash Method 4 and then defatted and dried according to the procedures described above. The feathers were then subjected to a reductive extraction in 8 M urea at pH 10 with 200 mM Tris, 3 mM EDTA, and 1.3 M thioglycolic acid (15 g feathers/1000 mL extraction buffer) at 60° C.-70° C. for 2 hours. The light pink extraction solution was filtered with a 100 μm filter, dialyzed against water for 3-5 days using dialysis tubing with a MWCO of 3.5 kDa, and the resulting cloudy solution was lyophilized for 3-5 days to give a dense off-white material and was then optionally ground into a fluffy off-white powder. Yields ranged from 30-40%. Extraction process 5 was also performed on feathers from Wash Methods 5 and 6, which yielded similar results.

Extraction Process 6: White chicken feathers were washed according to Wash Method 2c and then defatted and dried according to the procedures described above. The feathers were then subjected to a reductive extraction in 8 M urea at pH 10 with 200 mM Tris, 3 mM EDTA, and 1.3 M thioglycolic acid (15 g feathers/1000 mL extraction buffer) at 60° C.-70° C. for 2 hours. The light pink extraction solution was filtered with a 100 μm filter, dialyzed against water for 3-5 days using dialysis tubing with a MWCO of 3.5 kDa, and the resulting cloudy solution was lyophilized for 3-5 days to give a dense off-white material and was then optionally ground into a fluffy off-white powder. Yields ranged from 30-40%.

Extracts from the different processes above were studied in various tests, as described below. Most extracts were used to create keratin compositions. Extraction Process 4 gave such a low yield that only the solubility of the dried keratin extract was investigated. Indeed, the yield was so low that it would be impractical to prepare keratin compositions using keratin extracts obtained following Process 4.

### Example 4. Additional Purification of Keratin Extracts

In order to remove unwanted materials (for example, endotoxins, small molecules, lipids, surfactants, viral contaminants, unwanted proteins, unwanted tissue components, processing reagents, such as sodium dodecyl sulfate and thioglycolic acid, and so on), additional purification of the keratin extracts is performed.

(1) Buffer Exchange: Purification of various keratin extracts from Example 3 is achieved by exchanging the keratin extracts into a buffer solution and, in some instances, subsequently exchanging the extracts into water for further use. The exchange is accomplished by Tangential Flow Filtration, Dialysis, Depth Filtration, or Size Exclusion Chromatography.

(2) Chromatography: Various keratin extracts from Example 3 and extracts obtained after buffer exchange are subjected to chromatography accomplished by Ion Exchange Chromatography, Hydroxyapatite Chromatography, Hydrophobic Interaction Chromatography, or Mixed Mode Chromatography.

Certain keratin extracts that are purified by chromatography are subject to a second buffer exchange purification step to exchange the extracts into water or other solvents.

### Example 5. Molecular Weight Assessment of Keratin Extracts

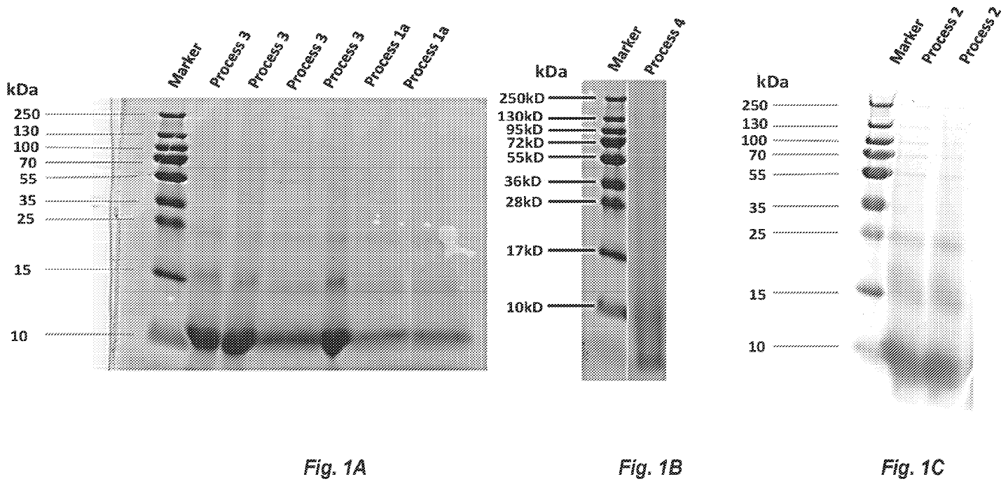

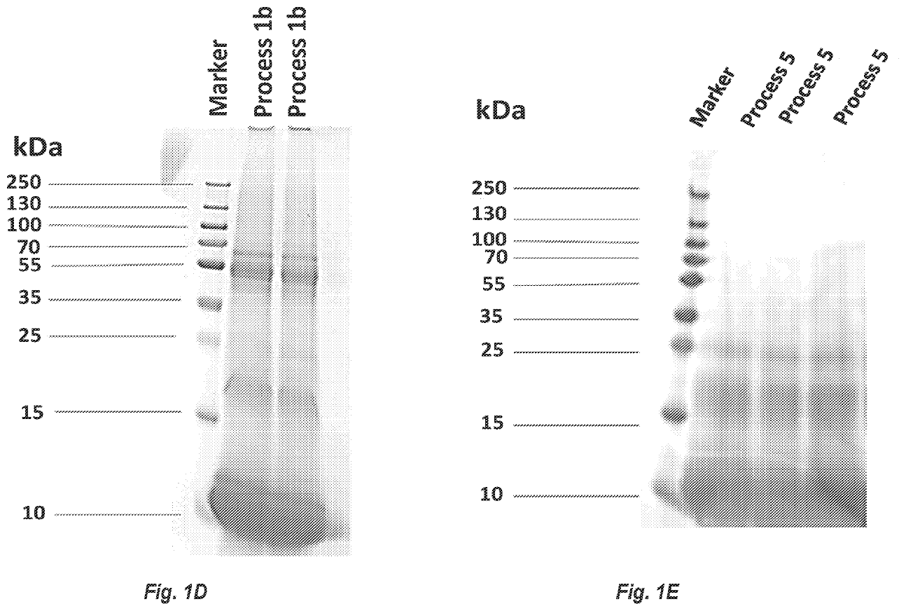

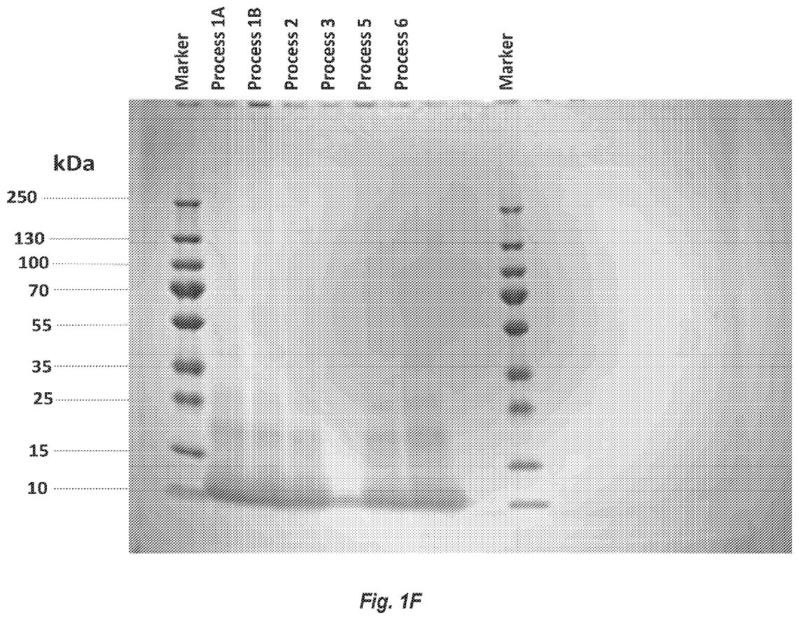

The purity of the keratin extracts from Example 3 (Extraction Processes 1-6) was assessed via SDS-PAGE to visualize the molecular weight of the extracted proteins. All processes yielded strong bands at ˜10 kDa, indicating a high purity of beta-keratins (FIGS. 1A-1F).

### Example 6. Thiol Content of Keratin Extracts

Keratin monomers and/or oligomers assemble in a network through physical crosslinking (e.g., hydrophobic interactions, ionic interactions), chemical crosslinking (e.g., disulfide bonds) or a combination thereof. Keratin networks created through physical interactions alone have been shown to degrade at an increased rate. As such, the availability of free thiol groups in a keratin extract is important in order to allow the formation of strong disulfide bonds in a network.

The amount of free thiols is even more important for beta-keratins because beta-keratin monomers are smaller than alpha-keratin monomers. This means the ability of beta-keratin monomers to crosslink through physical interactions may be limited. In addition, as discussed above, beta-keratin lacks KAPs that help form networks as in alpha-keratin.

The concentration of free thiols in certain keratin extracts obtained from Example 3 after dialysis but before lyophilization was measured using Thermo Scientific's Ellman's reagent and testing protocol. Ellman's reagent, or 5,5′-dithio-bis-(2-nitrobenzoic acid) (DTNB), is a water-soluble compound used for quantifying free sulfhydryl groups in solution. DTNB reacts with —SH groups to produce a yellow-colored product (TNB). Sulfhydryl groups can be quantified using a cysteine standard or based on molar absorptivity using the extinction coefficient of TNB equal to 14,150M⁻¹ cm⁻¹ at 412 nm.

Samples and standards were prepared in a reaction buffer consisting of 0.1M sodium phosphate, pH 8.0 and 1 mM EDTA. The w/v % of the keratin extract was determined by drying a fixed volume of the keratin extract and measuring the dry protein mass. Samples (250 μL) of 1% w/v and 0.1% w/v keratin extract in solution, as well as standard solutions, were then added to a solution of Ellman's reagent in 2.5 mL of reaction buffer and incubated for 15 minutes. Solution absorbance was measured at 412 nm.

Absorbance was directly correlated to known values using a cysteine standard curve provided by the manufacturer of the Ellman's reagent (Thermo Scientific).

The results are in Table 2 below. Extraction Processes 2, 5, and 6 gave average cysteine concentrations above 0.50 mM at 0.1% sample concentration, indicating that the extracts contain free thiols that are available for disulfide crosslinking. Extraction Processes 2, 5, and 6 also gave average cysteine concentrations above 0.50 mM at 1% sample concentration. For example, in one instance, Extraction Process 5 results in beta-keratin extracts with about 1.03 mM cysteine concentration at 1% w/v sample concentration.

TABLE 2

| | | | Cysteine | Average Cysteine |
|---|---|---|---|---|
| Extraction | Sample | | Concentration | Concentration |
| Process | Concentration | | (mM) | (mM) |
| Process 1b | 0.1% | w/v | 0.28-0.71 | 0.50 ± 0.10 mM (n = 3) |
| Process 2 | 0.1% | w/v | 0.49-0.71 | 0.57 ± 0.06 mM (n = 3) |
| Process 5 | 0.1% | w/v | 0.53-0.88 | 0.60 ± 0.06 mM (n = 5) |
| Process 6 | 0.1% | w/v | 0.38-0.62 | 0.52 ± 0.06 mM (n = 3) |
| Process 2 | 1% | w/v | 0.32-1.03 | 0.66 ± 0.17 mM (n = 3) |
| Process 5 | 1% | w/v | 0.77-1.29 | 0.97 ± 0.10 mM (n = 4) |
| Process 6 | 1% | w/v | 0.23-0.83 | 0.52 ± 0.14 mM (n = 3) |

Samples tested at increasing concentrations do not reflect a linear trend (i.e., increase in cysteine concentration) due to the nature of free thiol groups, which are prone to oxidation and crosslinking. Thus, the concentration of cysteine (mM cysteine) of a sample at 0.1% w/v cannot be extrapolated to determine the free thiol content (mM cysteine) of a sample at 1% w/v. However, a concentrated sample that is diluted (e.g., from 1% w/v to 0.1% w/v) is expected to have a lower concentration of cysteine (mol of cysteine/volume of sample) as the number of free thiols is not expected to increase even as the diluent volume does.

### Example 7. Physical Appearance of Keratin Extracts

The general physical appearance of the keratin extracts from the extraction processes in Example 3 are summarized in the table below.

TABLE 3

| | | | | Ground |
|---|---|---|---|---|
| | | Extraction | Lyophilized | lyophilized |
| Extraction | Extraction | (after | keratin | keratin |
| Process | Solution | dialysis) | extracts | extracts |
| Process 1a | Yellow | Dense white | Solid white | Dense |
| | solution | hydrogel | material | powder |
| Process 1b | Pale yellow | Cloudy | Powdery white | Dense |
| | solution | solution | material | powder |
| Process 2 | Light pink | Cloudy | Dense off- | Fluffy off- |
| | solution | solution | white material | white powder |
| Process 3 | Yellow | Disperse | White dense | N/A |
| | solution | aggregates | material | |
| | | in clear | | |
| | | solution | | |
| | | or dense | | |
| | | white | | |
| | | hydrogel | | |
| Process 4 | Slight purple | Cloudy | Dense white | N/A |
| | solution | solution | material | |
| Process 5 | Light pink | Cloudy | Dense off- | Fluffy off- |
| | solution | solution | white material | white powder |
| Process 6 | Light pink | Cloudy | Dense off- | Fluffy off- |
| | solution | solution | white material | white powder |

### Example 8. Solubility of Keratin Extracts

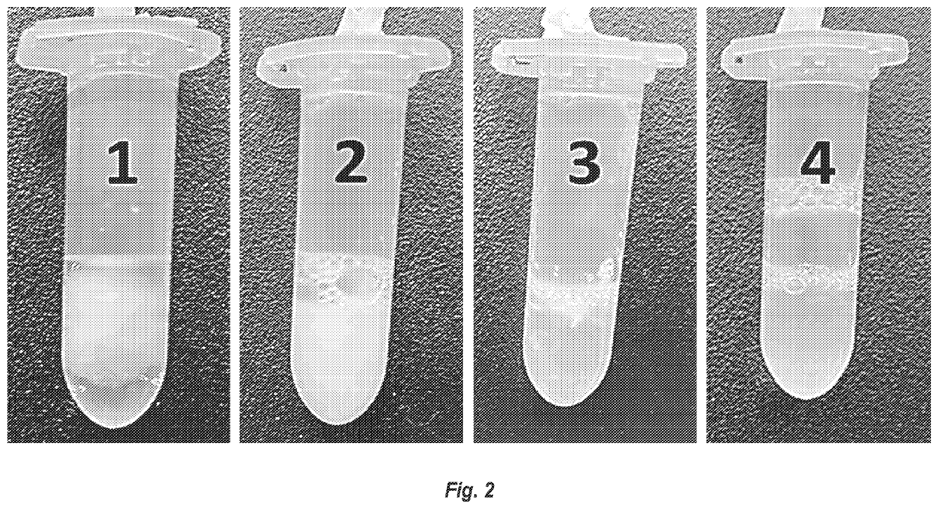

The lyophilized materials from Example 3 were combined with different solvents in order to test solubility as described herein. The lyophilized keratin extracts were combined with different solvents at 2.5% w/v and briefly agitated or vortexed. The lyophilized keratin extract from Process 2 was also tested at 5% w/v in water and briefly agitated or vortexed. Table 4 describes the rating system of solubility. FIG. 2 provides exemplary samples corresponding to each rating. Table 5 summarizes the solubility of the lyophilized keratin extracts in various solvents. A solubility rating of 1 or 2 indicates a lack of dispersion and homogeneity of the resulting solution. A solution rated 1-2 does not allow for further formulation (i.e., addition of additives to create a uniform solution). A solution rated 3-4 enables the addition of subsequent additives to form a homogenous keratin composition. Solubility ratings were assigned based on visual inspection with the naked eye.

As discussed above, while different extraction methods are known in the art, the resultant beta-keratin extracts are not soluble in neutral and near neutral pH solvents. Instead, beta-keratin extracts often require the use of strong acids or bases, which may cause skin irritation or a decrease in overall biocompatibility of a beta-keratin containing biomaterial. Additionally, the use of strong acids or bases may negatively alter the protein monomers and oligomers (and consequently the protein network) and subsequently inhibit the performance of the biomaterial. As shown in the table below, Extraction Processes 2, 4, 5 and 6 all result in beta-keratin extracts that are soluble in near neutral pH solvents at about 2.5% w/v. In addition, Extraction Processes 2, 5 and 6 result in beta-keratin extracts that are soluble in neutral pH solvents at about 2.5% w/v. The beta-keratin extract from Process 2 is soluble in neutral pH solvents even at 5% w/v.

TABLE 4
Solubility Rating System

| Rating | Description |
|---|---|
| 1 | Insoluble - insignificant amount of material goes into |
| | solution within about 5 minutes of being combined with a |
| | solvent |
| 2 | Significantly insoluble-a majority of sample remains in |
| | solid state, suspended in solution within about 5 minutes of |
| | being combined with a solvent |
| 3 | Slightly soluble - a cloudy, disperse solution within about |
| | 5 minutes of being combined with a solvent |
| 4 | Soluble - a solution with no visible material within about 5 |
| | minutes of being combined with a solvent; generally |
| | homogenous and optionally tinted |

TABLE 5
Solubility Rating of Bulk Material

| | | Solubility |
|---|---|---|
| Extraction Process | Solvent | Rating |
| Process 1a | H₂O | 1 |
| Process 1a | 6M NaOH, pH 14.77 | 4 |
| Process 1a | 1M NaOH, pH 14 | 4 |
| Process 1a | 0.1 mM NaOH (pH 10) | 3 |
| Process 1a | 0.001M HCl, pH 3 | 1 |
| Process 1a | 0.01M HCl, pH 1 | 1 |
| Process 1a | 1M HCl, pH 0 | 1 |
| Process 1b | H₂O | 2 |
| Process 1b | Formic acid | 4 |
| Process 1b | PBS, pH 9.6 | 3 |
| Process 2 (before milling) | Formic acid | 4 |
| Process 2 (before milling) | PBS, pH 7.9 | 3 |
| Process 2 (before milling) | H₂O | 3 |
| Process 2 (after milling) | H₂O | 4 |
| Process 2 (after milling) | PBS, pH 7 | 3 |
| Process 3 | H₂O | 2 |
| Process 4 | H₂O | 3 |
| Process 5 (after milling) | H₂O | 4 |
| Process 5(after milling) | PBS, pH 7 | 3 |
| Process 6 (after milling) | H₂O | 4 |
| Process 6 (after milling) | PBS, pH 7 | 3 |
| Process 2 (at 5% w/v, | H₂O | 4 |
| after milling) | | |

### Example 9. Endotoxin Levels in Keratin Extracts

The endotoxin levels of certain keratin extracts from Example 3 were measured using the LAL assay described above. In these cases, the lyophilized keratin extract was prepared at no more than 1 g/50 mL of endotoxin free water. All results were normalized to the mass of the test article (EU/mg of lyophilized keratin extract). The results are shown in the table below. As shown below, Extraction Process 5, which employed a multi-step washing method before extraction, demonstrated reduced endotoxin levels compared to Process 1b and Process 2.

TABLE 6

| Extraction Process | Endotoxin Levels | |
|---|---|---|
| Process 1a | 1.0-3.5 | EU/mg (n = 3) |
| Process 1b | 0.8-49.8 | EU/mg (n = 4) |
| Process 2 | 0.9-82.5 | EU/mg (n = 4) |
| Process 3 | 1.0-4.4 | EU/mg (n = 4) |
| Process 5 | 0.2-1.5 | EU/mg (n = 4) |
| Process 6 | 0.6-0.9 | EU/mg (n = 3) |

### Example 10. Proliferation Assays on Keratin Extracts and Compositions

(1) BrdU Assay: The proliferative effects of certain keratin extracts are studied in an in vitro cell proliferation assay known as the Bromodeoxyuridine (BrdU) assay. The assay detects DNA synthesis and cell proliferation. Proliferating cells incorporate the BrdU compound during DNA synthesis, which can then be detected in daughter cells. A stimulation index is used to compare untreated cells to cells treated with materials to be evaluated.

In the BrdU assay, cells are serum-starved prior to treatment with sample groups (e.g., keratin extracts). The keratin extract is then mixed with serum-free cell culture media and used to replace the serum-free media treatment. Cell culture media containing serum is added to the control group. Samples with a stimulation index greater than one are considered to have a proliferative effect. A proliferative effect is seen from certain keratin extracts.

(2) WS1 Human Fibroblast Assay: The proliferative effect of certain keratin extracts and compositions was studied using WS1 Human Fibroblast cells. Dried keratin extracts from Extraction Process 5 (Example 3) and dried keratin compositions using keratin extracts from Extraction Process 5 (Example 3) were placed in wells of a 24-well cell culture plate. Concentration ranges for keratin extracts in the keratin compositions were about 1.0% to about 2.5% w/v extract to solvent. Concentration ranges for PEG (of various molecular weights) in the keratin compositions were from about 0% to about 50% w/w PEG to extract. Concentration ranges for glycerol in the keratin compositions were from about 0% to about 60% w/w glycerol to extract.

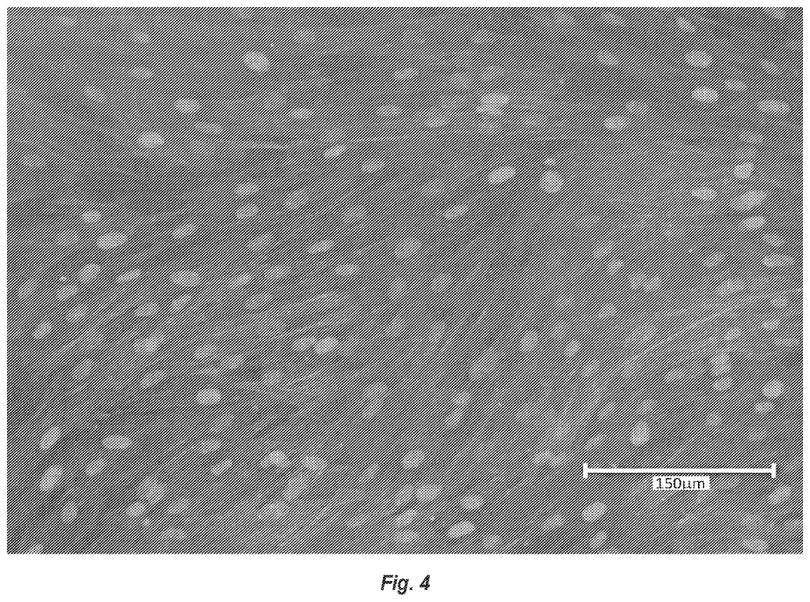

WS1 cells were seeded (5,000 cells/well) on the extracts and compositions. Cells were imaged 24 and 48 hours after seeding using phalloidin staining (to show cell body) and DAPI staining (to show cell nuclei) or brightfield imaging to show the cell body. Images were analyzed to determine the number of cells in each well at each time point. WS1 cells have a doubling time of approximately 18-24 hours. The tested extracts and compositions showed approximate doubling of cell number from 24 to 48 hours which is expected of cells proliferating at the anticipated rate indicating that the keratin extracts and compositions allow for proliferation of WS1 cells. Fluorescent staining of cells after 48 hours shows adhesion of cells to a keratin extract (FIG. 4—cell nuclei appear as gray ovals).

### Example 11. Preparation of Keratin Powder Compositions Using Keratin Extracts from Process 5

To prepare a keratin composition in the form of a powder, keratin extracts from Extraction Process 5 (Example 3) were combined with various concentrations of polyethylene glycol (PEG), glycerol, and salts. More specifically, keratin extracts from Extraction Process 5 in water (i.e., after dialysis but before lyophilization) were combined with additives and then lyophilized and milled. Keratin extracts in the extraction buffer (i.e., before dialysis in water) were combined with additives, dialyzed against water or other compatible solution containing additives, lyophilized, and milled.

Concentration ranges for keratin extracts were about 0.1 to about 1% w/v extract to solvent. Concentration ranges for PEG (of various molecular weights) were from about 0 to about 50% w/w PEG to extract. Concentration ranges for glycerol were from about 0 to about 60% w/w glycerol to extract. Salts included sodium chloride (NaCl) and sodium phosphate (Na₂HPO₄) at a concentration of from 0 mM to 100 mM.

### Example 12. Preparation of Keratin Sheet Compositions Using Keratin Extracts from Processes 1, 2, 5, and 6

To prepare keratin compositions in the form of a sheet, the lyophilized keratin extracts needed to be solubilized. Keratin extracts from Extraction Process 1 (Example 3) were solubilized in 1M NaOH at 65° C. The pH of the solution was then adjusted to pH 9-10 with 2M HCl. Keratin extracts from Process 2 (before or after milling) and keratin extracts from Processes 5 and 6 were mixed with water, PBS at pH 7, or PBS at pH 7.9 at room temperature.

The solutions were then combined with various concentrations of polyethylene glycols (PEG), carboxymethyl cellulose (CMC), and/or glycerol by weight. Concentration ranges for keratin extracts were about 1.5%, 2.5% to about 5% w/v extract to solvent. Concentration ranges for PEG (of various molecular weights) were from about 0% to about 70% w/w PEG to extract. Concentration ranges for glycerol were from about 0% to about 60% w/w glycerol to extract.

The resulting compositions were poured onto a set size casting surface and dried at 45° C. or room temperature to create sheets of varying thickness from 0.3-1.3 mm. Exemplary compositions that were prepared are shown in the table below.

After drying, the compositions were optionally milled to form a powder.

TABLE 7

| Composition # | PEG w/w % | Glycerol w/w % |
|---|---|---|
| 1 | 0 | 0 |
| 2 | 0 | 10 |
| 3 | 0 | 20 |
| 4 | 0 | 40 |
| 5 | 0 | 60 |
| 6 | 10 | 0 |
| 7 | 10 | 10 |
| 8 | 10 | 20 |
| 9 | 50 | 0 |
| 10 | 50 | 10 |
| 11 | 50 | 20 |
| 12 | 50 | 40 |
| 13 | 50 | 60 |
| 14 | 70 | 0 |
| 15 | 70 | 40 |
| 16 | 70 | 60 |
| 17 | 0.1 | 20 |
| 18 | 0.5 | 0 |
| 19 | 50 | 30 |
| 20 | 50 | 50 |
| 21 | 0 | 50 |
| 22 | 25 | 50 |
| 23 | 40 | 30 |
| 24 | 40 | 0 |
| 25 | 40 | 50 |
| 26 | 40 | 60 |
| 27 | 70 | 50 |
| 28 | 60 | 50 |
| 29 | 30 | 50 |

### Example 13. FTIR on Keratin Compositions

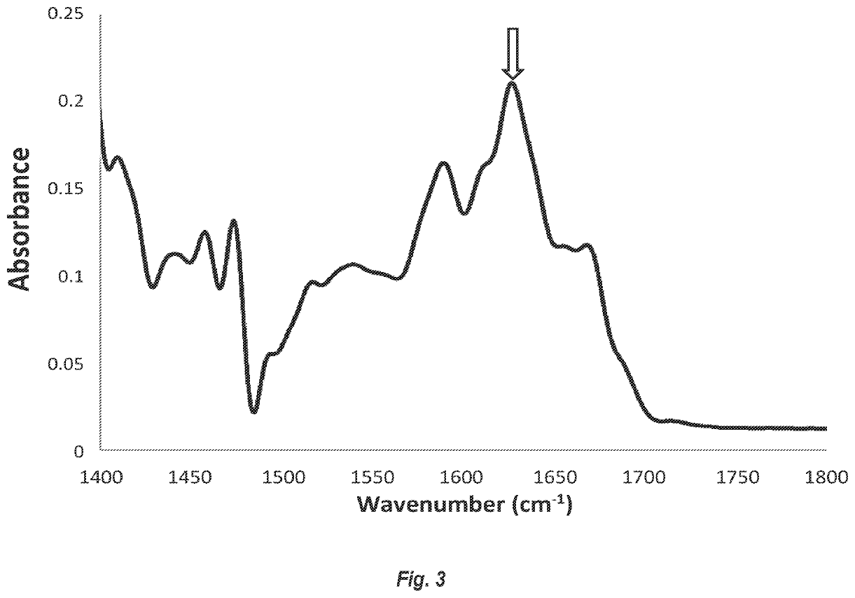

Sheets made from the extract from Extraction Process 2 (Example 3) with thicknesses of 0.6-0.8 mm and a keratin concentration of about 2.5% w/v were analyzed using FTIR to determine if a keratin network was formed. No crosslinking agents were used. Preliminary FTIR analysis showed that all tested keratin sheets contained the characteristic secondary structural elements, with the highest percentage structure being attributed to beta-sheet structures. Because no crosslinking agents were used, the beta-network is formed through intermolecular disulfide bonds. FIG. 3 shows an example spectrum of a keratin sheet prepared using keratin extracts prepared by Extraction Process 2. The FTIR analysis shows predominantly beta-sheet secondary structure, which is characteristic of beta-keratin monomers, oligomers, and filaments, as evidenced by the peak around 1630 cm⁻¹.

### Example 14. Endotoxin Levels of Keratin Compositions

The endotoxin levels of keratin sheet compositions prepared from the keratin extracts from Extraction Processes 2 and 6 (Example 3) were measured using the LAL assay described above. In these cases, sheets ranging from 4-20 cm² with a thickness of 0.6-0.8 mm were prepared and then tested in 50 mL of endotoxin free water. All results were normalized to the surface area of the test article (EU/cm²). The results are shown in the table below. Sheets prepared with keratin extracts from Extraction Process 6, which utilized a multi-step wash method before extraction, demonstrated reduced endotoxin levels compared to Process 2, which employed a single washing step.

TABLE 8

| Keratin Composition (Sheet) | Endotoxin Levels | |
|---|---|---|
| Prepared with keratin extracts from | 1.76 and 2.36 | EU/cm² (n = 2) |
| Extraction Process 6 (2.5% w/v keratin) | | |
| Prepared with keratin extracts from | 203 | EU/cm² (n = 1) |

Extraction Process 2 (5% w/v keratin)

### Example 15. Biocompatibility of Keratin Extracts

The biocompatibility of keratin extracts from Extraction Process 1a, 1b, 2, 3, 5, and 6 (Example 3) were evaluated for cytotoxicity using a qualitative evaluation of cell viability using BALB/3T3 embryonic mouse fibroblasts. The keratin extracts, after lyophilization, were incubated in Dulbecco's Modified Eagle Medium (DMEM) at 10 mg/mL for 24 hours at 37° C., followed by centrifugation to separate the lyophilized extract and media. The supernatant (100% conditioned media) was collected and applied to the cells. After a 24-hour incubation period, the cells were evaluated microscopically and assigned a cytotoxicity grade of 0-4 based on cell death and cell morphology following ISO 10993-12 guidelines from 2009, incorporated herein by reference. A cytotoxicity grade of ≤2 means the material is non-cytotoxic. All extracts were determined to be non-cytotoxic (Table 9).

TABLE 9

| Extraction | Cytotoxicity Grade | Cytotoxicity Grade | Pass/Fail |
|---|---|---|---|
| Process | (Trial 1) | (Trial 2) | (Trial 1/Trial 2) |
| 1a | 1 | 1 | Pass/Pass |
| 1b | 2 | 2 | Pass/Pass |
| 2 | 1 | 2 | Pass/Pass |
| 3 | 1 | 1 | Pass/Pass |
| 5 | 0 | 1 | Pass/Pass |
| 6 | 2 | 1 | Pass/Pass |

### Example 16. Biocompatibility of Keratin Compositions

The biocompatibility of keratin compositions can depend on the quality of the keratin extracts used to create the compositions. Keratin sheets made from Extraction Process 2 (Example 3) were investigated, as described in more detail below. PEG and/or glycerol were used in the compositions. Both are known biocompatible compounds.

The biocompatibility of keratin sheets produced from the keratin extracts obtained from Extraction Process 2 was evaluated using a MTT cell assay for cytotoxicity with BALB/3T3 embryonic mouse fibroblasts. The MTT assay is a colorimetric assay used for measuring cell viability and proliferation via metabolic activity. This standard cytotoxicity assay determines the viability of cells by measuring the number of metabolically active cells after the specified time of contact with complete media prepared from test material. Viable cells convert 3-[4,5-dimethylthiazol-2-yl]-2,5 diphenyl tetrazolium bromide (MTT) to formazan and the density of the color is read spectrophotometrically and compared to control cells, which are maintained in cell culture media.

Keratin sheets ranging in thickness from 0.6-0.7 mm with 2.5% w/v keratin were prepared following ISO 10993-12 guidelines from 2009, incorporated herein by reference, on the preparation of materials for biocompatibility testing. The keratin sheets were incubated in cell culture media at 6 cm²/mL for 24 hours at 37° C., followed by centrifugation to separate the sheet and media and collection of the supernatant (100% conditioned media). Test materials that result in greater than or equal to 70% cell viability are considered non-cytotoxic (see ISO 10993-5 from 2009, incorporated herein by reference). All test sheets evaluated were found to be non-cytotoxic in the MTT assay as cell viability was ≥70%.

### Example 17. Moisture Vapor Transmission Rate of Keratin Compositions

Maintaining a moist wound environment is essential for proper wound healing. Permeability of wound care devices has been shown to aid in efficient moisture management of wounds. See Xu, R. et al. “Controlled water vapor transmission rate promotes wound-healing via wound re-epithelialization and contraction enhancement,” Sci. Rep. 6, 24596; doi: 10.1038/srep24596 (2016).

Keratin compositions were prepared from keratin extracts of Extraction Process 2, glycerol and/or PEG with thicknesses ranging from 0.6-0.8 mm. The moisture vapor transmission rate (MVTR) of those keratin compositions was tested according to the ASTM E96 Cup Test Method, which is incorporated herein by reference. In short, a cup is filled with distilled water leaving a small gap of air space between the specimen and the water. The cup is then sealed to prevent vapor loss except through the test sample. An initial weight is taken of the apparatus and then periodically weighed over time until results become linear.

Measurements were taken at 37° C. with a relative humidity of 50%. The results of these studies showed that the keratin sheets made from 1.5-2.5% w/v keratin extract to casting solution, 0-50% w/w PEG to extract, and 60% w/w glycerol to extract have average MVTRs ranging from 3730 g/m²*day to 4426 g/m²*day (n=3). This indicates that the tested sheets have a high to extremely high permeability.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

What is claimed is:

1. A protein extract comprising beta-keratin monomers, wherein the extract has less than about 10.0 endotoxin units per mg of extract, such as less than about 0.80 endotoxin units per mg of extract.

2. A protein extract comprising beta-keratin monomers, wherein the extract has about 0.50 mM thiol groups when measured at about 0.1% weight extract per volume using Ellman's assay or above 0.20 mM thiol groups when measured at about 1% weight extract per volume using Ellman's assay.

3. A protein extract comprising beta-keratin monomers, wherein the extract when dehydrated is soluble or slightly soluble in a solvent having a pH from about 7 to about 8 at about 2.5% weight per volume.

4. The protein extract according to any one of claims 1-2, wherein the extract when dehydrated is soluble or slightly soluble in a solvent having a pH from about 7 to about 8 at about 2.5% weight per volume.

5. The protein extract according to claim 1 or 4, wherein the extract has about 0.50 mM thiol groups when measured at about 0.1% weight per volume using Ellman's assay or above 0.20 mM thiol groups when measured at about 1% weight extract per volume using Ellman's assay.

6. The protein extract according to any one of claims 1-5, wherein the extract has less than about 8.0 endotoxin units per mg of extract, such as less than about 0.10 endotoxin units per mg of extract.

7. The protein extract according to any one of claims 1-6, wherein the extract is lyophilized.

8. The protein extract according to any one of claims 3-7, wherein the extract is soluble and the solvent is water.

9. The protein extract according to any one of claims 1-8, wherein the extract when dehydrated is soluble in water at about 5% weight per volume.

10. The protein extract according to any one of claims 3-7 and 9, wherein the solvent having a pH from about 7 to about 8 is chosen from water and a PBS buffer having a pH from about 7 to about 8.

11. The protein extract according to any one of claims 1-10, wherein the extract comprises beta-keratin oligomers.

12. The protein extract according to any one of claims 1-11, wherein the extract does not comprise cysteine modified keratin monomers, oligomers, and/or filaments.

13. A method of extracting beta-keratin monomers comprising mixing a beta-keratin source material with an extraction solution comprising urea at a concentration of at least about 1 M and thioglycolic acid.

14. The method according to claim 13, wherein the urea concentration is at least about 5 M.

15. The method according to claim 13 or 14, wherein the urea concentration is from about 5 M to about 9 M.

16. The method according to any one of claims 13-15, wherein the urea concentration is from about 7 M to about 8 M.

17. The method according to any one of claims 13-16, wherein the urea concentration is about 8 M.

18. The method according to any one of claims 13-17, wherein the thioglycolic acid is at a concentration of about 0.5 M to about 1.5 M.

19. The method according to any one of claims 13-18, wherein the thioglycolic acid is at a concentration of about 1.3 M.

20. The method according to any one of claims 13-19, wherein the pH of the extraction solution is from about 7 to about 10.

21. The method according to any one of claims 13-20, wherein the pH of the extraction solution is about 10.

22. The method according to any one of claims 13-21, wherein the ratio of beta-keratin source material to extraction solution is, or is equivalent to, from about 1 g to about 100 g dry weight beta-keratin source material per about 1000 mL extraction solution.

23. The method according to any one of claims 13-22, wherein the ratio of beta-keratin source material to extraction solution is, or is equivalent to, about 15 g dry weight beta-keratin source material per about 1000 mL extraction solution.

24. The method according to any one of claims 13-23, wherein the extraction solution further comprises a chelating agent.

25. The method according to any one of claims 13-24, wherein the beta-keratin source material is mixed with the extraction solution for at least about 30 minutes.

26. The method according to any one of claims 13-25, wherein the temperature of the mixture of beta-keratin source material and the extraction solution is from about room temperature to about 80° C.

27. The method according to any one of claims 13-26, wherein the method further comprises washing the beta-keratin source material with a washing solution and/or sterilizing the beta-keratin source material prior to mixing the beta-keratin source material with the extraction solution.

28. The method according to any one of claims 13-27, wherein the method further comprises defatting the beta-keratin source material prior to mixing the beta-keratin source material with the extraction solution.

29. The method according to claim 27 or 28, wherein the washing solution comprises at least one detergent.

30. The method according to claim 29, wherein the at least one detergent comprises SDS and/or a nonionic detergent.

31. The method according to claim 30, wherein the nonionic detergent comprises a polyethylene glycol-based detergent.

32. The method according to claim 29 or 30, wherein the nonionic detergent comprises a polyethylene glycol tert-octylphenyl ether.

33. The method according to any one of claims 30-32, wherein the nonionic detergent comprises a compound of formula I: 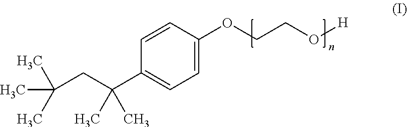
wherein n is chosen from 7-8, 9-10 or 40.

34. The method according to claim 33, wherein n is 7-8.

35. The method according to claim 33, wherein n is 9-10.

36. The method according to any one of claims 13-35, wherein the beta-keratin source material comprises an avian source material, for example goose and/or chicken feathers, and/or a reptilian source material, for example alligator scales.

37. The method according to any one of claims 13-36, wherein the extracted beta-keratin protein has less than about 10.0 endotoxin units per mg of extracted beta-keratin protein, such as less than about 0.80 endotoxin units per mg of extracted beta-keratin protein.

38. The method according to any one of claims 13-37, wherein the extracted beta-keratin protein has about 0.50 mM free thiol groups when measured at about 0.1% weight per volume using Ellman's assay or above 0.20 mM thiol groups when measured at about 1% weight extract per volume using Ellman's assay.

39. The method according to any one of claims 13-38, wherein the extracted beta-keratin protein when dehydrated is soluble or slightly soluble in a solvent having a pH from about 7 to about 8 at about 2.5% weight per volume.

40. The method according to claim 39, wherein the extract is soluble and the solvent is water.

41. The method according to any one of claims 13-40, wherein the extracted beta-keratin protein is produced in a yield from about 10% to about 70%, for example from about 30% to about 40% yield.

42. A method of preparing a beta-keratin protein extract comprising filtering the product of any one of claims 13-41 to produce a filtrate.

43. The method according to claim 42, wherein the method further comprises dialyzing the filtrate to produce a dialyzed material.

44. The method according to claim 42 or 43, wherein the method further comprises lyophilizing the filtrate or the dialyzed material to produce a lyophilized material.

45. The method according to claim 44, wherein the method further comprises homogenizing the lyophilized material.

46. The method according to any one of claims 13-45, wherein the method does not include modifying the cysteine residues of the beta-keratin source material and/or the extracted beta-keratin material.

47. A beta-keratin protein extract prepared according to any one of claims 13-46, wherein the beta-keratin protein extract has less than about 10.0 endotoxin units per mg of extract, such as less than about 0.80 endotoxin units per mg of extracted beta-keratin protein.

48. A beta-keratin protein extract prepared according to the method of any one of claims 13-47, wherein the beta-keratin protein extract has about 0.50 mM free thiol groups when measured at about 0.1% weight per volume using Ellman's assay or above 0.20 mM thiol groups when measured at about 1% weight extract per volume using Ellman's assay.

49. A beta-keratin protein extract prepared according to any one of claims 13-48, wherein the beta-keratin protein extract when dehydrated is soluble or slightly soluble in a solvent having a pH from about 7 to about 8 at about 2.5% weight per volume.

50. The beta-keratin protein extract according to claim 49, wherein the extract is soluble and the solvent is water.

51. A keratin composition comprising a beta-keratin extract, wherein the keratin composition has less than about 400 endotoxin units per composition.

52. A keratin composition comprising a beta-keratin network, wherein the keratin composition has less than about 400 endotoxin units per composition.

53. The keratin composition according to claim 51 or 52, wherein the keratin composition has less than about 20.0 endotoxin units per composition.

54. A keratin composition comprising a beta-keratin extract, wherein the keratin composition has less than about 400 endotoxin units per mg of the composition.

55. A keratin composition comprising a beta-keratin network, wherein the keratin composition has less than about 400 endotoxin units per mg of the composition.

56. The keratin composition according to claim 54 or 55, wherein the keratin composition has less than about 20.0 endotoxin units per mg of the composition.

57. A keratin composition comprising a beta-keratin extract, wherein the keratin composition has less than about 400 endotoxin units per cm².

58. A keratin composition comprising a beta-keratin network, wherein the keratin composition has less than about 400 endotoxin units per cm².

59. The keratin composition according to claim 57 or 58, wherein the keratin composition has less than about 20.0 endotoxin units per cm².

60. A keratin composition comprising a beta-keratin extract, wherein the keratin composition has less than about 400 endotoxin units per cm³.

61. A keratin composition comprising a beta-keratin network, wherein the keratin composition has less than about 400 endotoxin units per cm³.

62. The keratin composition according to claim 60 or 61, wherein the keratin composition has less than about 20.0 endotoxin units per cm³.

63. A keratin composition comprising a beta-keratin network, wherein the keratin composition has a moisture vapor transmission rate that is at least about 1000 g/(m²*day) of material per day.

64. The keratin composition according to any one of claim 52, 53, 55, 56, 58, 59 or 61-63, wherein the keratin composition has a moisture vapor transmission rate that is at least about 1000 g/(m²*day) of material per day.

65. A keratin composition comprising a beta-keratin network, wherein the keratin composition has a thickness of greater than about 0.2 mm.

66. The keratin composition according to any one of claim 52, 53, 55, 56, 58, 59 or 61-65, wherein the keratin composition has a thickness of greater than about 0.2 mm.

67. The keratin composition according to claim 65 or 66, wherein the keratin composition has a thickness ranging from about 0.2 mm to about 1 mm.

68. The keratin composition according to any one of claims 51-67, wherein the keratin composition comprises at least about 15 mg/cm³ of beta-keratin monomers, oligomers, and/or filaments.

69. The keratin composition according to any one of claim 52, 53, 55, 56, 58, 59 or 61, wherein the keratin composition is a continuous material.

70. The keratin composition according to claim 69, wherein the continuous material is in a form chosen from a sheet, a gel, a fiber and a foam.

71. The keratin composition according to claim 70, wherein the continuous material is a sheet that is fenestrated.

72. A keratin composition comprising a beta-keratin extract, wherein the keratin composition has less than about 400 endotoxin units per about 1 mL of the composition.

73. A keratin composition comprising a beta-keratin network, wherein the keratin composition has less than about 400 endotoxin units per about 1 mL of the composition.

74. The keratin composition according to claim 72 or 73, wherein the keratin composition has less than about 20.0 endotoxin units per about 1 mL of the composition.

75. The keratin composition according to any one of claim 51-56 or 72-74, wherein the keratin composition is in a form chosen from a dispersion and particles, such as a powder.

76. The keratin composition according to claim 75, wherein the dispersion is a suspension.

77. The keratin composition according to claim 75, wherein the keratin composition is in the form of a particle having an average particle size range of from about 8 nm to about 10 mm.

78. The keratin composition according to any one of claims 51-77, wherein the keratin composition is free of crosslinking agents.

79. The keratin composition according to any one of claims 51-78, wherein the keratin composition further comprises at least one additive.

80. The keratin composition according to claim 79, wherein the at least one additive is chosen from a protein, a carbohydrate, a polymer, a plasticizer, an antimicrobial agent, an anti-scarring agent, an anti-inflammatory agent, a pro-inflammatory agent, an angiogenic agent, a cell, a bulking agent, a stabilizer, a proteoglycan and a polysaccharide.

81. The keratin composition according to any one of claims 51-80, wherein the keratin composition comprises at least one plasticizer and at least one polymer.

82. The keratin composition according to claim 80 or 81, wherein the protein is chosen from an alpha-keratin monomer, oligomer and/or filament, a keratin-associated protein, a collagen, an elastin, a glycoprotein, a growth factor, a cytokine, silk, laminin, an antibody, an antimicrobial peptide, an integrin, an enzyme, an interleukin and a proteoglycan.

83. The keratin composition according to any one of claims 80-82, wherein the plasticizer is glycerol.

84. The keratin composition according to any one of claims 80-83, wherein the polymer is polyethylene glycol.

85. The keratin composition according to any one of claims 51-82 and 84, wherein the keratin composition does not comprise glycerol.

86. The keratin composition according to any one of claims 51-85, wherein the keratin composition further comprises alpha-keratin monomers, oligomers and/or filament.

87. The keratin composition according to any one of claims 51-86, wherein the keratin composition allows for cells to attach to the composition.

88. The keratin composition according to any one of claims 51-87, wherein the keratin composition allows for cell proliferation.

89. A method of preparing a keratin composition comprising combining a protein extract according to any one of claims 1-12 and 47-50 with at least one additive to give a keratin composition.

90. The method of claim 89, further comprising mixing the protein extract with a solvent prior to combining with the at least one additive.

91. The method of claim 90, wherein the solvent comprises water, a buffer, ethanol, methanol, poly(vinyl alcohol), formic acid, isopropanol, hexane and/or HFIP.

92. The method according to any one of claims 89-91, wherein the method further comprises casting the keratin composition onto a surface, such as a mold.

93. The method according to any one of claims 89-92, wherein the method further comprises freezing the keratin composition.

94. The method according to any one of claims 89-93, wherein the method further comprises dehydrating the keratin composition.

95. The method according to any one of claims 89-94, wherein the method further comprises cutting and/or milling the keratin composition.

96. The method according to any one of claims 89-91, wherein the method further comprises spinning the keratin composition into a keratin fiber.

97. The method of claim 96, wherein the method further comprises fabricating keratin fibers into a sheet.

98. The method according to any one of claims 89-97, wherein the method further comprises crosslinking the keratin composition.

99. The method according to any one of claims 89-98, wherein the crosslinking is chemical and/or physical crosslinking.

100. The method according to claim 98 or 99, wherein the crosslinking is a combination of chemical and physical crosslinking.

101. The method of claim 100, wherein the chemical crosslinking is achieved through chemical reaction of complementary groups, crosslinking with high energy radiation, enzymatic crosslinking, free radical polymerization, crosslinking with chemical crosslinkers, photo crosslinking, and/or UV crosslinking.

102. The method according to any one of claims 89-101, wherein the at least one additive is chosen from a protein, a carbohydrate, a polymer, a plasticizer, an antimicrobial agent, an anti-scarring agent, an anti-inflammatory agent, a pro-inflammatory agent, angiogenic agent, a cell, a bulking agent, a stabilizer, a proteoglycan and a polysaccharide, such as hyaluronic acid.

103. The method composition according to any one of claims 89-102, wherein the keratin composition comprises at least one plasticizer and at least one polymer.

104. The method according to claim 102 or 103, wherein the protein is chosen from an alpha-keratin monomer, oligomer and/or filament, a keratin-associated protein, a collagen, an elastin, a glycoprotein, a growth factor, a cytokine, silk, laminin, an antibody, an antimicrobial peptide, an integrin, an enzyme, an interleukin and a proteoglycan.

105. The method according to any one of claims 102-104, wherein the plasticizer is glycerol.

106. The method according to any one of claims 102-105, wherein the polymer is polyethylene glycol.

107. The method according to any one of claims 89-104 and 106, wherein the keratin composition does not comprise glycerol.

108. A keratin composition prepared according to any one of claims 89-107.

109. A method of treating a wound of a subject comprising administering to the wound the keratin composition according to any one of claim 51-88 or 108, or a protein extract according to any one of claim 1-12 or 47-50.

110. The method according to claim 109, wherein the treating comprises healing the wound, closing the wound and/or reducing signs of scars.

111. A method of managing a wound of a subject comprising covering a wound, absorbing exudate from a wound, and/or maintaining appropriate moisture balance within a wound with the keratin composition according to any one of claim 51-88 or 108, or a protein extract according to any one of claim 1-12 or 47-50.

112. The method according to any one of claims 109-111, wherein the wound is an acute or chronic wound chosen from an abrasion, laceration, skin tear, tunneled wound, incision, draining wound, ulcer and/or burn.

113. The method according to any one of claims 109-112, wherein the wound is a wound of the dermis, soft tissue, connective tissue, eye, nerve, gum, dura or bone.

114. A method for protecting a tissue of a subject comprising administering the keratin composition according to any one of claim 51-88 or 108, or a protein extract according to any one of claim 1-12 or 47-50 to the tissue.

115. The method according to claim 114, wherein the tissue is chosen from the dermis, soft tissue, connective tissue, eye, nerve, dura, gum and bone.

116. The method according to any one of claims 109-115, wherein the keratin composition or protein extract is applied to the surface of the wound or the tissue.

117. The method according to any one of claims 109-116, wherein the protein extract is dehydrated.

118. A method for delivering a pharmaceutically active agent to a subject in need thereof, comprising administering to the subject the keratin composition according to any one of claim 51-88 or 108, wherein the keratin composition further comprises the pharmaceutically active agent.

119. The method according to claim 118, wherein the pharmaceutically active agent is chosen from a protein, an antimicrobial agent, an anti-scarring agent, an anti-inflammatory agent, a pro-inflammatory agent, a small molecule, an angiogenic factor, a gene therapy and a cell.

120. The method according to claim 119, wherein the protein is chosen from an alpha-keratin monomer, oligomer and/or filament, a keratin-associated protein, a collagen, an elastin, a glycoprotein, a growth factor, a cytokine, silk, laminin, an antibody, an antimicrobial peptide, an integrin, an enzyme, an interleukin and a proteoglycan.

121. A kit comprising the protein extract according to any one of claim 1-12 or 47-50.

122. A kit comprising the keratin composition according to any one of claim 51-88 or 108.

123. The kit according to claim 121 or 122, wherein the kit further comprises a syringe.

124. The kit according to any one of claims 121-123, wherein the kit further comprises sutures.

125. The kit according to any one of claims 121-124, wherein the kit further comprises a wound dressing, an adhesive material, a non-adhesive material, an antimicrobial agent, a debriding agent, or saline.

126. A method of cleaning a beta-keratin source material comprising:
a) combining a beta-keratin source material with a first solution comprising a first ionic detergent;
b) soaking and/or agitating the beta-keratin source material in the first solution;
c) rinsing the beta-keratin source material with water;
d) soaking and/or agitating the beta-keratin source material in a second solution comprising a second ionic detergent;
e) rinsing the beta-keratin source material with water; and
f) optionally sterilizing the beta-keratin source material.

127. The method according to claim 126, wherein the method further comprises a step of drying the beta-keratin source material before step (d).

128. The method according to claim 126 or 127, wherein the first ionic detergent and/or second ionic detergent comprises sodium dodecyl sulfate.

129. A method of cleaning a beta-keratin source material comprising:
a) combining a beta-keratin source material with a first solution comprising at least about 0.1% v/v of a polyethylene glycol-based detergent in water; and
b) soaking and/or agitating the beta-keratin source material in the first solution.

130. The method according to claim 129, wherein the method further comprises a step (c) rinsing the beta-keratin source material with water and optionally drying the rinsed beta-keratin source material, and optionally sterilizing the rinsed or dried beta-keratin source material.

131. The method according to claim 130, wherein the method further comprises a step (d) soaking and/or agitating the beta-keratin source material in a second solution comprising a second detergent.

132. The method according to claim 131, wherein the method further comprises a step (e) rinsing the beta-keratin source material with water, optionally drying the rinsed beta-keratin source material, and optionally sterilizing the rinsed or dried beta-keratin source material.

133. The method according to claim 131 or 132, wherein the second detergent comprises a polyethylene glycol-based detergent.

134. A method of cleaning a beta-keratin source material comprising:
a) combining a beta-keratin source material with a first solution comprising a polyethylene glycol-based detergent;
b) soaking and/or agitating the beta-keratin source material in the first solution;
c) rinsing the beta-keratin source material with water and optionally drying the beta-keratin source material;
d) soaking and/or agitating the beta-keratin source material in a second solution comprising a polyethylene glycol-based detergent;
e) rinsing the beta-keratin source material with water; and
f) optionally sterilizing the beta-keratin source material.

135. The method according to any one of claims 129-134, wherein the polyethylene glycol-based detergent in step (a) comprises a polyethylene glycol tert-octylphenyl ether.

136. The method according to any one of claims 129-135, wherein the polyethylene glycol-based detergent in step (a) comprises a compound of formula (I): 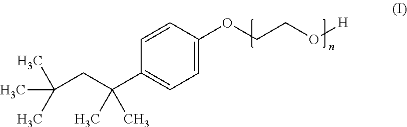
wherein n is chosen from 7-8, 9-10 or 40.

137. The method according to any one of claims 131-136, wherein the detergent in step (d) comprises a polyethylene glycol tert-octylphenyl ether.

138. The method according to any one of claims 131-136, wherein the detergent in step (d) comprises a compound of formula (I): 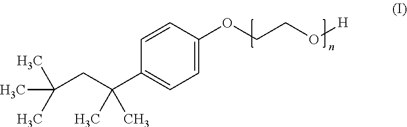
wherein n is chosen from 7-8, 9-10 or 40.

139. The method according to any one of claims 134-138, wherein the first solution in step (a) comprises at least about 0.1% v/v of a polyethylene glycol-based detergent in water.

140. The method according to any one of claims 131-139, wherein the second solution in step (d) comprises at least about 0.1% v/v of a polyethylene glycol-based detergent in water.

141. The method according to any one of claims 126-140, wherein the beta-keratin source material comprises an avian source material, for example goose feathers and/or chicken feathers, and/or a reptilian source material, for example alligator scales.

142. The method according to any one of claims 126-141, wherein the cleaned beta-keratin source material has less than about 3.25 endotoxin units per mg dry weight of cleaned beta-keratin source material.

143. The method according to any one of claims 126-142, wherein the cleaned beta-keratin source material has less than about 3.0 endotoxin units per mg dry weight of cleaned beta-keratin source material.

144. A cleaned beta-keratin source material prepared according to any one of claims 126-143.
